(19) United States
(12) Patent Application Publication
(10) Pub. No.: US 2002/0073443 A1
(43) Pub. Date: Jun. 13, 2002

(54) Herbicide tolerance achieved through plastid transformation

(76) Inventors: Peter B. Heifetz, San Diego, CA (US); Sandra L. Volrath, Durham, NC (US); Marie A. Johnson, Wendell, NC (US); Eric R. Ward, Durham, NC (US)

Correspondence Address: SYNGENTA BIOTECHNOLOGY, INC., PATENT DEPARTMENT, 3054 CORNWALLIS ROAD, P.O. BOX 12257, RESEARCH TRIANGLE PARK, NC, 27709-2257, US

(21) Appl. No.: 09/730,917

(22) Filed: Dec. 6, 2000

Related U.S. Application Data
(63) Continuation of application No. 09/059,164, filed on Apr. 13, 1998, now abandoned.
Application No. 09/059,164 is a continuation-in-part of application No. 09/050,603, filed on Mar. 30, 1998, now Pat. No. 6,023,012.
Application No. 09/050,603 is a continuation-in-part of application No. 08/808,931, filed on Feb. 28, 1997, now Pat. No. 5,939,602.
(60) Provisional application No. 60/012,705, filed on Feb. 28, 1996.
(60) Provisional application No. 60/013,612, filed on Feb. 28, 1996.
(60) Provisional application No. 60/020,003, filed on Jun. 21, 1996.
(60) Provisional application No. 60/126,430, filed on Mar. 11, 1998.

(51) Int. Cl. C12N 15/82

(52) U.S. Cl. 800/278

(57) ABSTRACT

Disclosed are novel DNA sequences isolated from soybean, wheat, cotton, sugar beet, rape, rice, sorghum, and sugar cane encode enzymes having protoporphyrinogen oxidase (protox) activity. In addition, modified, herbicide-tolerant forms of protox enzymes are disclosed. Plants expressing herbicide-tolerant protox enzymes taught herein are also provided. These plants may be engineered for resistance to protox inhibitors via mutation of the native protox gene to a resistant form or they may be transformed with a gene encoding an inhibitor-resistant from of a plant protox enzyme. Further disclosed is a method of achieving herbicide tolerance through plastid transformation, which involves transforming plastids with a chimeric gene that comprises a plastid-active promoter operatively linked to a DNA molecule that encodes a plastid-targeted enzyme whose native plastid transit peptide is either mutated so as to be nonfunctional or is absent altogether. The encoded enzyme may be for example a herbicide-tolerant protox enzyme, whereby the resulting transplastomic plants are resistant to protox inhibitors.

### CROSS-REFERENCE TO RELATED APPLICATIONS

This application is a continuation-in-part of U.S. application Ser. No. 09/050,603, filed Mar. 30, 1998, which is a continuation-in-part of U.S. application Ser. No. 08/808,931, filed Feb. 28, 1997, which itself claims the benefit of U.S. Provisional Application No. 60/012,705, filed Feb. 28, 1996, U.S. Provisional Application No. 60/013,612, filed Feb. 28, 1996, and U.S. Provisional Application No. 60/020,003, filed Jun. 21, 1996. This application is also a continuation-in-part of U.S. application Ser. No. 09/038,878, filed Mar. 11, 1998. All of the aforementioned applications are incorpororated herein by reference in their entireties.

### FIELD OF THE INVENTION

The invention relates generally to molecular biology and relates more specifically to transformation of plastids such as those in plants. In particular, the invention relates to the confering of herbicide tolerance in plants through plastid transformation. DNA molecules encoding modified, inhibitor-resistant forms of essential plant enzymes such as protoporphyrinogen oxidase (“protox”) are provided, as well as chimeric genes, vectors, plastids, and whole plants comprising such DNA molecules. The invention further relates to methods for tissue culture selection and herbicide application utilizing the DNA molecules of the invention.

### BACKGROUND OF THE INVENTION

I. The Protox Enzyme and its Involvement in the Chlorophyll/Heme Biosynthetic Pathway

The biosynthetic pathways that lead to the production of chlorophyll and heme share a number of common steps. Chlorophyll is a light harvesting pigment present in all green photosynthetic organisms. Heme is a cofactor of hemoglobin, cytochromes, P450 mixed-function oxygenases, peroxidases, and catalyses (see, e.g. Lehninger, Biochemistry. Worth Publishers, New York (1975)), and is therefore a necessary component for all aerobic organisms.

The last common step in chlorophyll and heme biosynthesis is the oxidation of protoporphyrinogen IX to protoporphyrin IX. Protoporphyrinogen oxidase (referred to herein as “protox”) is the enzyme that catalyzes this last oxidation step (Matringe et al., Biochem. J. 260:231 (1989)).

The protox enzyme has been purified either partially or completely from a number of organisms including the yeast Saccharomyces cerevisiae (Labbe-Bois and Labbe, In Biosynthesis of Heme and Chlorophyll, E. H. Dailey, ed. McGraw Hill: New York, pp. 235-285 (1990)), barley etioplasts (Jacobs and Jacobs, Biochem. J. 244: 219 (1987)), and mouse liver (Dailey and Karr, Biochem. 26: 2697 (1987)). Genes encoding protox have been isolated from two prokaryotic organisms, Escherichia coil (Sasarman et al., Can. J. Microbiol. 39:1155 (1993)) and Bacillus subtilis (Dailey et al., J. Biol. Chem. 269: 813 (1994)). These genes share no sequence similarity; neither do their predicted protein products share any amino acid sequence identity. The E. coli protein is approximately 21 kDa, and associates with the cell membrane. The B. subtilis protein is 51 kDa, and is a soluble, cytoplasmic activity.

Protox encoding genes have now also been isolated from humans (see Nishimura et al., J. Biol. Chem. 270(14): 8076-8080 (1995) and plants (International application no. PCT/IB95/00452 filed Jun. 8, 1995, published Dec. 21, 1995 as WO 95/34659).

II. The Protox Gene as a Herbicide Target

The use of herbicides to control undesirable vegetation such as weeds or plants in crops has become an almost universal practice. The relevant market exceeds a billion dollars annually. Despite this extensive use, weed control remains a significant and costly problem for farmers.

Effective use of herbicides requires sound management. For instance, time and method of application and stage of weed plant development are critical to getting good weed control with herbicides. Since various weed species are resistant to herbicides, the production of effective herbicides becomes increasingly important. Novel herbicides can now be discovered using high-throughput screens that implement recombinant DNA technology. Metabolic enzymes essential to plant growth and development can be recombinantly produced though standard molecular biological techniques and utilized as herbicide targets in screens for novel inhibitors of the enzymes' activity. The novel inhibitors discovered through such screens may then be used as herbicides to control undesirable vegetation.

Unfortunately, herbicides that exhibit greater potency, broader weed spectrum and more rapid degradation in soil can also have greater crop phytotoxicity. One solution applied to this problem has been to develop crops that are resistant or tolerant to herbicides. Crop hybrids or varieties resistant to the herbicides allow for the use of the herbicides without attendant risk of damage to the crop. Development of resistance can allow application of a herbicide to a crop where its use was previously precluded or limited (e.g. to pre-emergence use) due to sensitivity of the crop to the herbicide. For example, U.S. Pat. No. 4,761,373, incorporated herein by reference, is directed to plants resistant to various imidazolinone or sulfonamide herbicides. The resistance is conferred by an altered acetohydroxyacid synthase (AHAS) enzyme. U.S. Pat. No. 4,975,374, incorporated herein by reference, relates to plant cells and plants containing a gene encoding a mutant glutamine synthetase (GS) resistant to inhibition by herbicides that were known to inhibit GS, e.g. phosphinothricin and methionine sulfoximine. U.S. Pat. No. 5,013,659, incorporated herein by reference, is directed to plants that express a mutant acetolactate synthase (ALS) that renders the plants resistant to inhibition by sulfonylurea herbicides. U.S. Pat. No. 5,162,602, incorporated herein by reference, discloses plants tolerant to inhibition by cyclohexanedione and aryloxyphenoxypropanoic acid herbicides. The tolerance is conferred by an altered acetyl coenzyme A carboxylase(ACCase). U.S. Pat. No. 5,554,798, incorporated herein by reference, discloses transgenic glyphosate resistant maize plants, which tolerance is conferred by an altered 5-enolpyruvyl-3-phosphoshikimate (EPSP) synthase gene.

The protox enzyme serves as the target for a variety of herbicidal compounds. The herbicides that inhibit protox include many different structural classes of molecules (Duke et al., Weed Sci. 39: 465 (1991); Nandihalli et al., Pesticide Biochem. Physiol. 43. 193 (1992); Matringe et al., FEBS Lett. 245: 35 (1989); Yanase and Andoh, Pesticide Biochem. Physiol. 35: 70 (1989)). These herbicidal compounds include the diphenylethers {e.g. acifluorfen, 5-[2-chloro-4-(trifluoromethyl)phenoxy]-2-nitrobezoic acid; its methyl ester; or oxyfluorfen, 2-chloro-1-(3-ethoxy-4-nitrophenoxy)-4-(trifluorobenzene)}, oxidiazoles, (e.g. oxidiazon, 3-[2,4-dichloro-5-(1-methylethoxy)phenyl]-5-(1,1-dimethylethyl)-1,3,4-oxadiazol-2-(3R-one), cyclic imides (e.g. S-23142,N-(4-chloro-2-fluoro-5-propargyloxyphenyl)-3,4,5,6-tetrahydrophthalimide; chlorophthalim, N-(4-chlorophenyl)-3,4,5,6-tetrahydrophthalimide), phenyl pyrazoles (e.g. TNPP-ethyl, ethyl 2-[1-(2,3,4-trichlorophenyl)-4-nitropyrazolyl-5-oxy]propionate; M&B 39279), pyridine derivatives (e.g. LS 82-556), and phenopylate and its O-phenylpyrrolidino- and piperidinocarbamate analogs. Many of these compounds competitively inhibit the normal reaction catalyzed by the enzyme, apparently acting as substrate analogs.

Typically, the inhibitory effect on protox is determined by measuring fluorescence at about 622 to 635 nm, after excitation at about 395 to 410 nM (see, e.g. Jacobs and Jacobs, Enzyme 28:206 (1982); Sherman et al., Plant Physiol. 97:280 (1991)). This assay is based on the fact that protoporphyrin IX is a fluorescent pigment, and protoporphyrinogen IX is nonfluorescent.

The predicted mode of action of protox-inhibiting herbicides involves the accumulation of protoporphyrinogen IX in the chloroplast. This accumulation is thought to lead to leakage of protoporphyrinogen IX into the cytosol where it is oxidized by a peroxidase activity to protoporphyrin IX. When exposed to light, protoporphyrin IX can cause formation of singlet oxygen in the cytosol. This singlet oxygen can in turn lead to the formation of other reactive oxygen species, which can cause lipid peroxidation and membrane disruption leading to rapid cell death (Lee et al., Plant Physiol. 102: 881 (1993)).

Not all protox enzymes are sensitive to herbicides that inhibit plant protox enzymes. Both of the protox enzymes encoded by genes isolated from Escherichia coli (Sasarman et al., Can. J. Microbiol. 39:1155 (1993)) and Bacillus subtilis (Dailey et al., J. Biol. Chem. 269: 813 (1994)) are resistant to these herbicidal inhibitors. In addition, mutants of the unicellular alga Chlamydomonas reinhardtii resistant to the phenylimide herbicide S-23142 have been reported (Kataoka et al., J. Pesticide Sci. 15: 449 (1990); Shibata et al., In Research in Photosynthesis, Vol. 111, N. Murata, ed. Kluwer:Netherlands. pp. 567-570 (1992)). At least one of these mutants appears to have an altered protox activity that is resistant not only to the herbicidal inhibitor on which the mutant was selected, but also to other classes of protox inhibitors (Oshio et al., Z. Naturforsch. 48c: 339 (1993); Sato et al., In ACS Symposium on Porphyric Pesticides, S. Duke, ed. ACS Press: Washington, D.C. (1994)). A mutant tobacco cell line has also been reported that is resistant to the inhibitor S-21432 (Che et al.,. Z. Naturforsch. 48c: 350 (1993).

III. Plastid Transformation and Expression

Plastid transformation, in which genes are inserted by homologous recombination into some or all of the several thousand copies of the circular plastid genome present in each plant cell, takes advantage of the enormous copy number advantage over nuclear-expressed genes to permit expression levels that may exceed 10% of the total soluble plant protein. In addition, plastid transformation is desirable because in most plants plastid-encoded traits are not pollen transmissible; hence, potential risks of inadvertent transgene escape to wild relatives of transgenic plants is obviated. Plastid transformation technology is extensively described in U.S. Pat. Nos. 5,451,513, 5,545,817, 5,545,818 and 5,576,198; in PCT application Nos. WO 95/16783 and WO 97/32977; and in McBride et al., Proc. Natl. Acad. Sci. USA 91: 7301-7305 (1994), all of which are incorporated herein by reference. Plastid transformation via biolistics was achieved initially in the unicellular green alga Chlamydomonas reinhardtii (Boynton et al. (1988) Science 240: 1534-1537, incorporated herein by reference) and this approach, using selection for cis-acting antibiotic resistance loci (spectinomycin/streptomycin resistance) or complementation of non-photosynthetic mutant phenotypes, was soon extended to Nicotiana tabacum (Svab et al. (1990) Proc. Natl. Acad. Sci. USA. 87: 8526-8530, incorporated herein by reference).

The basic technique for tobacco chloroplast transformation involves the particle bombardment of leaf tissue or PEG-mediated uptake of plasmid DNA in protoplasts with regions of cloned plastid DNA flanking a selectable antibiotic resistance marker. The 1 to 1.5 kb flanking regions, termed targeting sequences, facilitate homologous recombination with the plastid genome and thus allow the replacement or modification of specific regions of the 156 kb tobacco plastid genome. Initially, point mutations in the chloroplast 16S rDNA and rps12 genes conferring resistance to spectinomycin and/or streptomycin were utilized as selectable markers for transformation (Svab, Z., Hajdukiewicz, P., and Maliga, P. (1990) Proc. Natl. Acad. Sci. USA 87, 8526-8530; Staub, J. M., and Maliga, P. (1992) Plant Cell 4, 39-45, incorporated herein by reference). This resulted in stable homoplasmic transformants at a frequency of approximately one per 100 bombardments of target leaves. The presence of cloning sites between these markers allowed creation of a plastid targeting vector for introduction of foreign genes (Staub, J. M., and Maliga, P., EMBO J. 12: 601-606 (1993), incorporated herein by reference). Substantial increases in transformation frequency were obtained by replacement of the recessive rRNA or r-protein antibiotic resistance genes with a dominant selectable marker, the bacterial aadA gene encoding the spectinomycin-detoxifying enzyme aminoglycoside-3′-adenyltransferase (Svab, Z., and Maliga, P. (1993) Proc. Natl. Acad. Sci. USA 90, 913-917, incorporated herein by reference). Previously, this marker had been used successfully for high-frequency transformation of the plastid genome of the green alga Chlamydomonas reinhardtii(Goldschmidt-Clermont, M. (1991) Nucl. Acids Res. 19, 4083-4089, incorporated herein by reference). Recently, plastid transformation of protoplasts from tobacco and the moss Physcomitrella patens has been attained using polyethylene glycol (PEG) mediated DNA uptake (O'Neill et al. (1993) Plant J. 3: 729-738; Koop et al. (1996) Planta 199: 193-201, both of which are incorporated herein by reference).

### SUMMARY OF THE INVENTION

The present invention provides DNA molecules isolated from wheat, soybean, cotton, sugar beet, rape, rice, and sorghum encoding enzymes having protoporphyrinogen oxidase (protox) activity and chimeric genes comprising such DNA. Sequences of such DNA molecules are set forth in SEQ ID NOs: 9 (wheat), 11 (soybean), 15 (cotton), 17 (sugar beet), 19 (rape), 21 (rice), 23 (sorghum), and 36 (sugar cane).

The present invention also provides modified forms of plant protoporphyrinogen oxidase (protox) enzymes that are resistant to compounds that inhibit unmodified naturally occurring plant protox enzymes, and DNA molecules coding for such inhibitor-resistant plant protox enzymes. The present invention includes chimeric genes and modified forms of naturally occurring protox genes that can express the inhibitor-resistant plant protox enzymes in plants.

Genes encoding inhibitor-resistant plant protox enzymes can be used to confer resistance to protox-inhibitory herbicides in whole plants and as a selectable marker in plant cell transformation methods. Accordingly, the present invention also includes plants, including the descendants thereof, plant tissues and plant seeds containing plant expressible genes encoding these modified protox enzymes. These plants, plant tissues and plant seeds are resistant to protox-inhibitors at levels that normally are inhibitory to the naturally occurring protox activity in the plant. Plants encompassed by the invention especially include those that would be potential targets for protox inhibiting herbicides, particularly agronomically important crops such as maize and other cereal crops such as barley, wheat, sorghum, rye, oats, turf and forage grasses, millet and rice. Also comprised are other crop plants such as sugar cane, soybean, cotton, sugar beet, oilseed rape and tobacco.

The present invention is directed further to methods for the production of plants, including plant material, such as for example plant tissues, protoplasts, cells, calli, organs, plant seeds, embryos, pollen, egg cells, zygotes, together with any other propagating material and plant parts, such as for example flowers, stems, fruits, leaves, roots originating in transgenic plants or their progeny previously transformed by means of the process of the invention, which produce an inhibitor-resistant form of the plant protox enzyme provided herein. Such plants may be stably transformed with a structural gene encoding the resistant protox, or prepared by direct selection techniques whereby herbicide resistant lines are isolated, characterized and developed.

The present invention is further directed to probes and methods for detecting the presence of genes encoding inhibitor-resistant forms of the plant protox enzyme and quantitating levels of inhibitor-resistant protox transcripts in plant tissue. These methods may be used to identify or screen for plants or plant tissue containing and/or expressing a gene encoding an inhibitor-resistant form of the plant protox enzyme.

The present invention also relates to plastid transformation and to the expression of DNA molecules in a plant plastid. In a preferred embodiment, a native plant protox enzyme or a modified plant protox enzyme is expressed in plant plastids to obtain herbicide resistant plants.

In a further embodiment, the present invention is directed to a chimeric gene comprising: (a) a DNA molecule isolated from a plant, which in its native state encodes a polypeptide that comprises a plastid transit peptide, and a mature enzyme that is natively targeted to a plastid of the plant by the plastid transit peptide, wherein the DNA molecule is modified such that it does not encode a functional plastid transit peptide; and (b) a promoter capable of expressing the DNA molecule in a plastid, wherein the promoter is operatively linked to the DNA molecule. The DNA molecule may be modified in that at least a portion of the native plastid transit peptide coding sequence is absent from the DNA molecule. Alternatively, the DNA molecule may be modified in that one or more nucleotides of the native plastid transit peptide coding sequence are mutated, thereby rendering an encoded plastid transit peptide nonfunctional. The present invention also relates to plants homoplasmic for chloroplast genomes containing such chimeric genes. In a preferred embodiment, the DNA molecule encodes an enzyme that is naturally inhibited by a herbicidal compound. In this case, such plants are resistant to a herbicide that naturally inhibits the enzyme encoded by a DNA molecule according to the present invention.

The present invention is also directed to plants made resistant to a herbicide by transforming their plastid genome with a DNA molecule according to the present invention and to methods for obtaining such plants. In a preferred embodiment, the DNA molecule encodes an enzyme that is naturally inhibited by a herbicidal compound. In a more preferred embodiment, the DNA molecule encodes an enzyme having protoporphyrinogen oxidase (protox) activity, which is modified so that it that confers resistance to protox inhibitors. A further embodiment of the present invention is directed to a method for controlling the growth of undesired vegetation, which comprises applying to a population of the above-described plants an effective amount of an inhibitor of the enzyme.

The present invention also provides a novel method for selecting a transplastomic plant cell, comprising the steps of: introducing the above-described chimeric gene into the plastome of a plant cell; expressing the encoded enzyme in the plastids of said plant cell; and selecting a cell that is resistant to a herbicidal compound that naturally inhibits the activity of the enzyme, whereby the resistant cell comprises transformed plastids. In a preferred embodiment, the enzyme is naturally inhibited by a herbicidal compound and the transgenic plant is able to grow on an amount of the herbicidal compound that naturally inhibits the activity of the enzyme. In a further preferred embodiment, the enzyme has protoporphyrinogen oxidase (protox) activity and is modified so that it that confers resistance to protox inhibitors.

### DESCRIPTION OF THE SEQUENCE LISTING

SEQ ID NO:1: DNA coding sequence for an Arabidopsis thaliana protox-1 protein.

SEQ ID NO:2: Arabidopsis protox-1 amino acid sequence encoded by SEQ ID NO:1.

SEQ ID NO:3: DNA coding sequence for an Arabidopsis thaliana protox-2 protein.

SEQ ID NO:4: Arabidopsis protox-2 amino acid sequence encoded by SEQ ID NO:3.

SEQ ID NO:5: DNA coding sequence for a maize protox-1 protein.

SEQ ID NO:6: Maize protox-1 amino acid sequence encoded by SEQ ID NO:5.

SEQ ID NO:7: DNA coding sequence for a maize protox-2 protein.

SEQ ID NO:8: Maize protox-2 amino acid sequence encoded by SEQ ID NO:7.

SEQ ID NO:9: Partial DNA coding sequence for a wheat protox-1 protein.

SEQ ID NO:10: Partial wheat protox-1 amino acid sequence encoded by SEQ ID NO:9.

SEQ ID NO:11: DNA coding sequence for a soybean protox-1 protein.

SEQ ID NO:12: Soybean protox-1 protein encoded by SEQ ID NO:11.

SEQ ID NO:13: Promoter sequence from Arabidopsis thaliana protox-1 gene.

SEQ ID NO:14: Promoter sequence from maize protox-1 gene.

SEQ ID NO:15: DNA coding sequence for a cotton protox-1 protein.

SEQ ID NO:16: Cotton protox-1 amino acid sequence encoded by SEQ ID NO:15.

SEQ ID NO:17: DNA coding sequence for a sugar beet protox-1 protein.

SEQ ID NO:18: Sugar beet protox-1 amino acid sequence encoded by SEQ ID NO:17.

SEQ ID NO:19: DNA coding sequence for a rape protox-1 protein.

SEQ ID NO:20: Rape protox-1 amino acid sequence encoded by SEQ ID NO:19.

SEQ ID NO:21: Partial DNA coding sequence for a rice protox-1 protein.

SEQ ID NO:22: Partial rice protox-1 amino acid sequence encoded by SEQ ID NO:21.

SEQ ID NO:23: Partial DNA coding sequence for a sorghum protox-1 protein.

SEQ ID NO:24: Partial sorghum protox-1 amino acid sequence encoded by SEQ ID NO:23.

SEQ ID NO:25: Maize protox-1 intron sequence.

SEQ ID NO:26: Promoter sequence from sugar beet protox-1 gene.

SEQ ID NO:27: Pclp_P1a—plastid clpP gene promoter top strand PCR primer.

SEQ ID NO:28: Pclp_P1b—plastid clpP gene promoter bottom strand PCR primer.

SEQ ID NO:29: Pclp_P2b—plastid clpP gene promoter bottom strand PCR primer.

SEQ ID NO:30: Trps16_P1a—plastid rps16 gene top strand PCR primer.

SEQ ID NO:31: Trps16_p1b—plastid rps16 gene bottom strand PCR primer.

SEQ ID NO:32: minpsb_U—plastid psbA gene top strand primer.

SEQ ID NO:33: minpsb_L—plastid psbA gene bottom strand primer.

SEQ ID NO:34: APRTXP1a—top strand PCR primer.

SEQ ID NO:35: APRTXP1b—bottom strand PCR primer.

SEQ ID NO:36: Partial DNA coding sequence for a sugar cane protox-1 protein.

SEQ ID NO:37: Partial sugar cane protox-1 amino acid sequence encoded by SEQ ID NO:36.

### DEPOSITS

The following vector molecules have been deposited with Agricultural Research Service, Patent Culture Collection (NRRL), Northern Regional Research Center, 1815 North University Street, Peoria, Ill. 61604, U.S.A on the dates indicated below:

Wheat Protox-1a, in the pBluescript SK vector, was deposited Mar. 19, 1996, as pWDC-13 (NRRL #B21545).

Soybean Protox-1, in the pBluescript SK vector, was deposited Dec. 15, 1995 as pWDC-12 (NRRL #B-21516).

Cotton Protox-1, in the pBluescript SK vector, was deposited Jul. 1, 1996 as pWDC-15 (NRRL #B-21594).

Sugar beet Protox-1, in the pBluescript SK vector, was deposited Jul. 29, 1996, as pWDC-16 (NRRL #B-21595N).

Rape Protox-1, in the pBluescript SK vector, was deposited Aug. 23, 1996, as pWDC-17 (NRRL #B-21615).

Rice Protox-1, in the pBluescript SK vector, was deposited Dec. 6, 1996, as pWDC-18 (NRRL #B-21648).

Sorghum Protox-1, in the pBluescript SK vector, was deposited Dec. 6, 1996, as pWDC-19 (NRRL #B-21649).

Resistant mutant pAraC-2Cys, in the pMut-1 plasmid, was deposited on Nov. 14, 1994 under the designation pWDC-7 with the Agricultural Research Culture Collection and given the deposit designation NRRL #21339N.

AraPT1Pro containing the Arabidopsis Protox-1 promoter was deposited Dec. 15, 1995, as pWDC-11 (NRRL #B-21515)

A plasmid containing the maize Protox-1 promoter fused to the remainder of the maize Protox-1 coding sequence was deposited Mar. 19, 1996 as pWDC-14 (NRRL #B-21546).

A plasmid containing the Sugar Beet Protox-1 promoter was deposited Dec. 6, 1996, as pWDC-20 (NRRL #B-21650).

### DEFINITIONS

For clarity, certain terms used in the specification are defined and presented as follows:

Associated With/Operatively Linked: refers to two DNA sequences that are related physically or functionally. For example, a promoter or regulatory DNA sequence is said to be “associated with” a DNA sequence that codes for an RNA or a protein if the two sequences are operatively linked, or situated such that the regulator DNA sequence will affect the expression level of the coding or structural DNA sequence.

Chimeric Gene: a recombinant DNA sequence in which a promoter or regulatory DNA sequence is operatively linked to, or associated with, a DNA sequence that codes for an mRNA or which is expressed as a protein, such that the regulator DNA sequence is able to regulate transcription or expression of the associated DNA sequence. The regulator DNA sequence of the chimeric gene is not normally operatively linked to the associated DNA sequence as found in nature.

Coding DNA Sequence: a DNA sequence that is translated in an organism to produce a protein.

Herbicide: a chemical substance used to kill or suppress the growth of plants, plant cells, plant seeds, or plant tissues.

Heterologous DNA Sequence: a DNA sequence not naturally associated with a host cell into which it is introduced, including non-naturally occurring multiple copies of a naturally occurring DNA sequence.

Homologous DNA Sequence: a DNA sequence naturally associated with a host cell into which it is introduced.

Homoplasmic: refers to a plant, plant tissue or plant cell, wherein all of the plastids are genetically identical. In different tissues or stages of development, the plastids may take different forms, e.g., chloroplasts, proplastids, etioplasts, amyloplasts, chromoplasts, and so forth.

Inhibitor: a chemical substance that inactivates the enzymatic activity of a protein such as a biosynthetic enzyme, receptor, signal transduction protein, structural gene product, or transport protein that is essential to the growth or survival of the plant. In the context of the instant invention, an inhibitor is a chemical substance that inactivates the enzymatic activity of protox. The term “herbicide” is used herein to define an inhibitor when applied to plants, plant cells, plant seeds, or plant tissues.

Isolated: in the context of the present invention, an isolated DNA molecule or an isolated enzyme is a DNA molecule or enzyme that, by the hand of man, exists apart from its native environment and is therefore not a product of nature. An isolated DNA molecule or enzyme may exist in a purified form or may exist in a non-native environment such as, for example, a transgenic host cell.

Minimal Promoter: promoter elements, particularly a TATA element, that are inactive or that have greatly reduced promoter activity in the absence of upstream activation. In the presence of a suitable transcrption factor, the minimal promoter functions to permit transcription.

Modified Enzyme Activity: enzyme activity different from that which naturally occurs in a plant (i.e. enzyme activity that occurs naturally in the absence of direct or indirect manipulation of such activity by man), which is tolerant to inhibitors that inhibit the naturally occurring enzyme activity.

Plant: refers to any plant or part of a plant at any stage of development. Therein are also included cuttings, cell or tissue cultures and seeds. As used in conjunction with the present invention, the term “plant tissue” includes, but is not limited to, whole plants, plant cells, plant organs, plant seeds, protoplasts, callus, cell cultures, and any groups of plant cells organized into structural and/or functional units.

Plastome: the genome of a plastid.

Protox-1: chloroplast protox.

Protox-2: mitochondrial protox.

Significant Increase: an increase in enzymatic activity that is larger than the margin of error inherent in the measurement technique, preferably an increase by about 2-fold or greater of the activity of the wild-type enzyme in the presence of the inhibitor, more preferably an increase by about 5-fold or greater, and most preferably an increase by about 10-fold or greater.

Substantially Similar: with respect to nucleic acids, a nucleic acid molecule that has at least 60 percent sequence identity with a reference nucleic acid molecule. In a preferred embodiment, a substantially similar DNA sequence is at least 80% identical to a reference DNA sequence; in a more preferred embodiment, a substantially similar DNA sequence is at least 90% identical to a reference DNA sequence; and in a most preferred embodiment, a substantially similar DNA sequence is at least 95% identical to a reference DNA sequence. A substantially similar nucleotide sequence typically hybridizes to a reference nucleic acid molecule, or fragments thereof, under the following conditions: hybridization at 7% sodium dodecyl sulfate (SDS), 0.5 M NaPO₄ pH 7.0, 1 mM EDTA at 50° C.; wash with 2×SSC, 1% SDS, at 50° C. With respect to proteins or peptides, a substantially similar amino acid sequence is an amino acid sequence that is at least 90% identical to the amino acid sequence of a reference protein or peptide and has substantially the same activity as the reference protein or peptide.

Tolerance: the ability to continue normal growth or function when exposed to an inhibitor or herbicide.

Transformation: a process for introducing heterologous DNA into a cell, tissue, or plant. Transformed cells, tissues, or plants are understood to encompass not only the end product of a transformation process, but also transgenic progeny thereof.

Transit Peptide: a signal polypeptide that is translated in conjunction with a protein encoded by a DNA molecule, forming a polypeptide precursor. In the process of transport to a selected site within the cell, a chloroplast for example, the transit peptide can be cleaved from the remainder of the polypeptide precursor to provide an active or mature protein.

Transformed: refers to an organism such as a plant into which a heterologous DNA molecule has been introduced. The DNA molecule can be stably integrated into the genome of the plant, wherein the genome of the plant encompasses the nuclear genome, the plastid genome and the mitochondrial genome. In a transformed plant, the DNA molecule can also be present as an extrachromosomal molecule. Such an extrachromosomal molecule can be auto-replicating. A “non-transformed” plant refers to a wild-type organism, i.e., a plant, which does not contain the heterologous DNA molecule.

Transplastome: a transformed plastid genome.

### DETAILED DESCRIPTION OF THE INVENTION

I. Plant Protox Coding Sequences

In one aspect, the present invention is directed to an isolated DNA molecule that encodes protoporphyrinogen oxidase (referred to herein as “protox”), the enzyme that catalyzes the oxidation of protoporphyrinogen IX to protoporphyrin IX, from wheat, soybean, cotton, sugar beet, rape, rice, sorghum, and sugar cane. The partial DNA coding sequence and corresponding amino acid sequence for a wheat protox enzyme are provided as SEQ ID NOs:9 and 10, respectively. The DNA coding sequence and corresponding amino acid sequence for a soybean protox enzyme are provided as SEQ ID NOs:11 and 12, respectively. The DNA coding sequence and corresponding amino acid sequence for a cotton protox enzyme are provided as SEQ ID NOs:15 and 16, respectively. The DNA coding sequence and corresponding amino acid sequence for a sugar beet protox enzyme are provided as SEQ ID NOs:17 and 18, respectively. The DNA coding sequence and corresponding amino acid sequence for a rape protox enzyme are provided as SEQ ID NOs:19 and 20, respectively. The partial DNA coding sequence and corresponding amino acid sequence for a rice protox enzyme are provided as SEQ ID NOs:21 and 22, respectively. The partial DNA coding sequence and corresponding amino acid sequence for a sorghum protox enzyme are provided as SEQ ID NOs:23 and 24, respectively. The partial DNA coding sequence and corresponding amino acid sequence for a sugar cane protox enzyme are provided as SEQ ID NOs:36 and 37, respectively.

The DNA coding sequences and corresponding amino acid sequences for protox enzymes from Arabidopsis thaliana and maize that have been previously isolated are reproduced herein as SEQ ID NOs:1-4 (Arabidopsis) and SEQ ID NOs:5-8 (maize).

The invention therefore primarily is directed to a DNA molecule encoding a protoporphyrinogen oxidase (protox) comprising a eukaryotic protox selected from the group consisting of a wheat protox enzyme, a soybean protox enzyme, a cotton protox enzyme, a sugar beet protox enzyme, a rape protox enzyme, a rice protox enzyme and a sorghum protox enzyme.

Preferred within the scope of the invention are isolated DNA molecules encoding the protoporphyrinogen oxidase (protox) enzyme from dicotyledonous plants, but especially from soybean plants, cotton plants, sugar beet plants and rape plants, such as those given in SEQ ID NOS: 11, 15, 17 and 19. More preferred are isolated DNA molecules encoding the protoporphyrinogen oxidase (protox) enzyme from soybean, such as given in SEQ ID NO:11, and sugar beet, such as given in SEQ ID NO:17.

Also preferred are isolated DNA molecules encoding the protoporphyrinogen oxidase (protox) enzyme from monocotyledonous plants, but especially from wheat plants, rice plants, sorghum plants, and sugar cane plants, such as those given in SEQ ID NOS: 9, 21, 23, and 36. More preferred are isolated DNA molecules encoding the protoporphyrinogen oxidase (protox) enzyme from wheat such as given in SEQ ID NO:9.

In another aspect, the present invention is directed to isolated DNA molecules encoding the protoporphyrinogen oxidase (protox) enzyme protein from a dicotyledonous plant, wherein said protein comprises the amino acid sequence selected from the group consisting of SEQ ID NOs: 12, 16, 18 and 20. Further comprised are isolated DNA molecules encoding the protoporphyrinogen oxidase (protox) enzyme protein from a monocotyledonous plant, wherein said protein comprises the amino acid sequence selected from the group consisting of SEQ ID NOs: 10, 22, 24, and 37. More preferred is an isolated DNA molecule encoding the protoporphyrinogen oxidase (protox) enzyme wherein said protein comprises the amino acid sequence from wheat such as given in SEQ ID NO:10. More preferred is an isolated DNA molecule encoding the protoporphyrinogen oxidase (protox) enzyme wherein said protein comprises the amino acid sequence from soybean, such as given in SEQ ID NO:12 and sugar beet, such as given in SEQ ID NO:18.

Using the information provided by the present invention, the DNA coding sequence for the protoporphyrinogen oxidase (protox) enzyme from any eukaryotic organism may be obtained using standard methods.

In another aspect, the present invention is directed to an isolated DNA molecule that encodes a wheat protox enzyme and that comprises a nucleotide sequence that hybridizes to the coding sequence shown in SEQ ID NO:9 under the following hybridization and wash conditions:

(a) hybridization in 7% sodium dodecyl sulfate (SDS), 0.5 M NaPO4 pH 7.0, 1 mM EDTA at 5° C.; and

(b) wash in 2×SSC, 1% SDS at 50° C.

In yet another aspect, the present invention is directed to an isolated DNA molecule that encodes a soybean protox enzyme and that comprises a nucleotide sequence that hybridizes to the coding sequence shown in SEQ ID NO:11 under the following hybridization and wash conditions:

(a) hybridization in 7% sodium dodecyl sulfate (SDS), 0.5 M NaPO4 pH 7.0, 1 mM EDTA at 50° C.; and

(b) wash in 2×SSC, 1% SDS at 50° C.

In still another aspect, the present invention is directed to an isolated DNA molecule that encodes a cotton protox enzyme and that comprises a nucleotide sequence that hybridizes to the coding sequence shown in SEQ ID NO:15 under the following hybridization and wash conditions:

(a) hybridization in 7% sodium dodecyl sulfate (SDS), 0.5 M NaPO4 pH 7.0, 1 mM EDTA at 50° C.; and

(b) wash in 2×SSC, 1% SDS at 50° C.

In another aspect, the present invention is directed to an isolated DNA molecule that encodes a sugar beet protox enzyme and that comprises a nucleotide sequence that hybridizes to the coding sequence shown in SEQ ID NO:17 under the following hybridization and wash conditions:

(a) hybridization in 7% sodium dodecyl sulfate (SDS), 0.5 M NaPO4 pH 7.0, 1 mM EDTA at 50° C.; and

(b) wash in 2×SSC, 1% SDS at 50° C.

In another aspect, the present invention is directed to an isolated DNA molecule that encodes a rape protox enzyme and that comprises a nucleotide sequence that hybridizes to the coding sequence shown in SEQ ID NO:19 under the following hybridization and wash conditions:

(a) hybridization in 7% sodium dodecyl sulfate (SDS), 0.5 M NaPO4 pH 7.0, 1 mM EDTA at 50° C.; and

(b) wash in 2×SSC, 1% SDS at 50° C.

In another aspect, the present invention is directed to an isolated DNA molecule that encodes a rice protox enzyme and that comprises a nucleotide sequence that hybridizes to the coding sequence shown in SEQ ID NO:21 under the following hybridization and wash conditions:

(a) hybridization in 7% sodium dodecyl sulfate (SDS), 0.5 M NaPO4 pH 7.0, 1 mM EDTA at 50° C.; and

(b) wash in 2×SSC, 1% SDS at 50° C.

In another aspect, the present invention is directed to an isolated DNA molecule that encodes a sorghum protox enzyme and that comprises a nucleotide sequence that hybridizes to the coding sequence shown in SEQ ID NO:23 under the following hybridization and wash conditions:

(a) hybridization in 7% sodium dodecyl sulfate (SDS), 0.5 M NaPO4 pH 7.0, 1 mM EDTA at 50° C.; and

(b) wash in 2×SSC, 1% SDS at 50° C.

In another aspect, the present invention is directed to an isolated DNA molecule that encodes a sugar cane protox enzyme and that comprises a nucleotide sequence that hybridizes to the coding sequence shown in SEQ ID NO:36 under the following hybridization and wash conditions:

(a) hybridization in 7% sodium dodecyl sulfate (SDS), 0.5 M NaPO4 pH 7.0, 1 mM EDTA at 50° C.; and

(b) wash in 2×SSC, 1% SDS at 50° C.

The isolated eukaryotic protox sequences taught by the present invention may be manipulated according to standard genetic engineering techniques to suit any desired purpose. For example, the entire protox sequence or portions thereof may be used as probes capable of specifically hybridizing to protox coding sequences and messenger RNA's. To achieve specific hybridization under a variety of conditions, such probes include sequences that are unique among protox coding sequences and are preferably at least 10 nucleotides in length, and most preferably at least 20 nucleotides in length. Such probes may be used to amplify and analyze protox coding sequences from a chosen organism via the well known process of polymerase chain reaction (PCR). This technique may be useful to isolate additional protox coding sequences from a desired organism or as a diagnostic assay to determine the presence of protox coding sequences in an organism.

Factors that affect the stability of hybrids determine the stringency of the hybridization. One such factor is the melting temperature Tₘ, which can be easily calculated according to the formula provided in DNA PROBES, George H. Keller and Mark M. Manak, Macmillan Publishers Ltd, 1993, Section one: Molecular Hybridization Technology; page 8 ff. The preferred hybridization temperature is in the range of about 25° C. below the calculated melting temperature Tₘ and preferably in the range of about 12-15° C. below the calculated melting temperature Tₘ and in the case of oligonucleotides in the range of about 5-10° C. below the melting temperature Tₘ.

Comprised by the present invention are DNA molecules that hybridize to a DNA molecule according to the invention as defined hereinbefore, but preferably to an oligonucleotide probe obtainable from said DNA molecule comprising a contiguous portion of the sequence of the said protoporphyrinogen oxidase (protox) enzyme at least 10 nucleotides in length, under moderately stringent conditions.

The invention further embodies the use of a nucleotide probe capable of specifically hybridizing to a plant protox gene or mRNA of at least 10 nucleotides length in a polymerase chain reaction (PCR).

In a further embodiment, the present invention provides probes capable of specifically hybridizing to a eukaryotic DNA sequence encoding a protoporphyrinogen oxidase activity or to the respective mRNA and methods for detecting the said DNA sequences in eukaryotic organisms using the probes according to the invention.

Protox specific hybridization probes may also be used to map the location of the native eukaryotic protox gene(s) in the genome of a chosen organism using standard techniques based on the selective hybridization of the probe to genomic protox sequences. These techniques include, but are not limited to, identification of DNA polymorphisms identified or contained within the protox probe sequence, and use of such polymorphisms to follow segregation of the protox gene relative to other markers of known map position in a mapping population derived from self fertilization of a hybrid of two polymorphic parental lines (see e.g. Helentjaris et al., Plant Mol. Biol. 5: 109 (1985). Sommer et al. Biotechniques 12:82 (1992); D'Ovidio et al., Plant Mol. Biol. 15: 169 (1990)). While any eukaryotic protox sequence is contemplated to be useful as a probe for mapping protox genes from any eukaryotic organism, preferred probes are those protox sequences from organisms more closely related to the chosen organism, and most preferred probes are those protox sequences from the chosen organism. Mapping of protox genes in this manner is contemplated to be particularly useful in plants for breeding purposes. For instance, by knowing the genetic map position of a mutant protox gene that confers herbicide resistance, flanking DNA markers can be identified from a reference genetic map (see, e.g., Helentjaris, Trends Genet. 3: 217 (1987)). During introgression of the herbicide resistance trait into a new breeding line, these markers can then be used to monitor the extent of protox-linked flanking chromosomal DNA still present in the recurrent parent after each round of back-crossing.

Protox specific hybridization probes may also be used to quantitate levels of protox mRNA in an organism using standard techniques such as Northern blot analysis. This technique may be useful as a diagnostic assay to detect altered levels of protox expression that may be associated with particular adverse conditions such as autosomal dominant disorder in humans characterized by both neuropsychiatric symptoms and skin lesions, which are associated with decreased levels of protox activity (Brenner and Bloomer, New Engl. J. Med. 302:765 (1980)).

A further embodiment of the invention is a method of producing a DNA molecule comprising a DNA portion encoding a protein having protoporphyrinogen oxidase (protox) enzyme activity comprising:

(a) preparing a nucleotide probe capable of specifically hybridizing to a plant protox gene or mRNA, wherein said probe comprises a contiguous portion of the coding sequence for a protox protein from a plant of at least 10 nucleotides length;

(b) probing for other protox coding sequences in populations of cloned genomic DNA fragments or cDNA fragments from a chosen organism using the nucleotide probe prepared according to step (a); and

(c) isolating and multiplying a DNA molecule comprising a DNA portion encoding a protein having protoporphyrinogen oxidase (protox) enzyme activity.

A further embodiment of the invention is a method of isolating a DNA molecule from any plant comprising a DNA portion encoding a protein having protoporphyrinogen oxidase (protox) enzyme activity.

(a) preparing a nucleotide probe capable of specifically hybridizing to a plant protox gene or mRNA, wherein said probe comprises a contiguous portion of the coding sequence for a protox protein from a plant of at least 10 nucleotides length;

(b) probing for other protox coding sequences in populations of cloned genomic DNA fragments or cDNA fragments from a chosen organism using the nucleotide probe prepared according to step (a); and

(c) isolating a DNA molecule comprising a DNA portion encoding a protein having protoporphyrinogen oxidase (protox) enzyme activity.

The invention further comprises a method of producing an essentially pure DNA sequence coding for a protein exhibiting protoporphyrinogen oxidase (protox) enzyme activity, which method comprises:

(a) preparing a genomic or a cDNA library from a suitable source organism using an appropriate cloning vector;

(b) hybridizing the library with a probe molecule; and

(c) identifying positive hybridizations of the probe to the DNA clones from the library that is clones potentially containing the nucleotide sequence corresponding to the amino acid sequence for protoporphyrinogen oxidase (protox).

The invention further comprises a method of producing an essentially pure DNA sequence coding for a protein exhibiting protoporphyrinogen oxidase (protox) enzyme activity, which method comprises:

(a) preparing total DNA from a genomic or a cDNA library;

(b) using the DNA of step (a) as a template for PCR reaction with primers representing low degeneracy portions of the amino acid sequence of protoporphyrinogen oxidase (protox).

A further object of the invention is an assay to identify inhibitors of protoporphyrinogen oxidase (protox) enzyme activity that comprises:

(a) incubating a first sample of protoporphyrinogen oxidase (protox) and its substrate;

(b) measuring an uninhibited reactivity of the protoporphyrinogen oxidase (protox) from step (a);

(c) incubating a first sample of protoporphyrinogen oxidase (protox) and its substrate in the presence of a second sample comprising an inhibitor compound;

(d) measuring an inhibited reactivity of the protoporphyrinogen oxidase (protox) enzyme from step (c); and

(e) comparing the inhibited reactivity to the uninhibited reactivity of protoporphyrinogen oxidase (protox) enzyme.

A further object of the invention is an assay to identify inhibitor-resistant protoporphyrinogen oxidase (protox) mutants that comprises:

(a) incubating a first sample of protoporphyrinogen oxidase (protox) enzyme and its substrate in the presence of a second sample comprising a protoporphyrinogen oxidase (protox) enzyme inhibitor;

(b) measuring an unmutated reactivity of the protoporphyrinogen oxidase (protox) enzyme from step (a);

(c) incubating a first sample of a mutated protoporphyrinogen oxidase (protox) enzyme and its substrate in the presence of a second sample comprising protoporphyrinogen oxidase (protox) enzyme inhibitor;

(d) measuring a mutated reactivity of the mutated protoporphyrinogen oxidase (protox) enzyme from step (c); and

(e) comparing the mutated reactivity to the unmutated reactivity of the protoporphyrinogen oxidase (protox) enzyme.

A further object of the invention is a protox enzyme inhibitor obtained by a method according to the invention.

For recombinant production of the enzyme in a host organism, the protox coding sequence may be inserted into an expression cassette designed for the chosen host and introduced into the host where it is recombinantly produced. The choice of specific regulatory sequences such as promoter, signal sequence, 5′ and 3′ untranslated sequences, and enhancer, is within the level of skill of the routineer in the art. The resultant molecule, containing the individual elements linked in proper reading frame, may be inserted into a vector capable of being transformed into the host cell. Suitable expression vectors and methods for recombinant production of proteins are well known for host organisms such as E. coli (see, e.g. Studier and Moffatt, J. Mol. Biol. 189: 113 (1986); Brosius, DNA 8: 759 (1989)), yeast (see, e.g., Schneider and Guarente, Meth. Enzymol. 194: 373 (1991)) and insect cells (see, e.g., Luckow and Summers, Bio/Technol. 6: 47 (1988)). Specific examples include plasmids such as pBluescript (Stratagene, La Jolla, Calif.), pFLAG (International Biotechnologies, Inc., New Haven, Conn.), pTrcHis (Invitrogen, La Jolla, Calif.), and baculovirus expression vectors, e.g., those derived from the genome of Autographica califonica nuclear polyhedrosis virus (AcMNPV). A preferred baculovirus/insect system is pVl11392/Sf21 cells (Invitrogen, La Jolla, Calif.).

Recombinantly produced eukaryotic protox enzyme is useful for a variety of purposes. For example, it may be used to supply protox enzymatic activity in vitro. It may also be used in an in vitro assay to screen known herbicidal chemicals whose target has not been identified to determine if they inhibit protox. Such an in vitro assay may also be used as a more general screen to identify chemicals that inhibit protox activity and that are therefore herbicide candidates. Recombinantly produced eukaryotic protox enzyme may also be used in an assay to identify inhibitor-resistant protox mutants (see International application No. PCT/IB95/00452 filed Jun. 8, 1995, published Dec. 21, 1995 as WO 95/34659, incorporated by reference herein in its entirety). Alternatively, recombinantly produced protox enzyme may be used to further characterize its association with known inhibitors in order to rationally design new inhibitory herbicides as well as herbicide tolerant forms of the enzyme.

II. Inhibitor Resistant Plant Protox Enzymes

In another aspect, the present invention teaches modifications that can be made to the amino acid sequence of any plant protoporphyrinogen oxidase (referred to herein as “protox”) enzyme to yield an inhibitor-resistant form of this enzyme. The present invention is directed to inhibitor-resistant plant protox enzymes having the modifications taught herein, and to DNA molecules encoding these modified enzymes, and to genes capable of expressing these modified enzymes in plants.

The present invention is thus directed to an isolated DNA molecule encoding a modified protoporphyrinogen oxidase (protox) having at least one amino acid modification, wherein said amino acid modification having the property of conferring resistance to a protox inhibitor, that is wherein said modified protox is tolerant to a herbicide in amounts that inhibit said eukaryotic protox. As used herein ‘inhibit’ refers to a reduction in enzymatic activity observed in the presence of a subject herbicide compared to the level of activity observed in the absence of the subject herbicide, wherein the percent level of reduction is preferably at least 10%, more preferably at least 50%, and most preferably at least 90%.

Preferred is a DNA molecule encoding a modified protoporphyrinogen oxidase (protox) comprising a eukaryotic protox selected from the group consisting of a wheat protox enzyme, a soybean protox enzyme, a cotton protox enzyme, a sugar beet protox enzyme, a rape protox enzyme, a rice protox enzyme and a sorghum protox enzyme having at least one amino acid modification, wherein said modified protox is tolerant to a herbicide in amounts that inhibit the naturally occurring protox activity.

Also preferred is a DNA molecule encoding a modified protoporphyrinogen oxidase (protox) comprising a plant protox wherein the cysteine occurring at the position corresponding to amino acid 159 of SEQ ID NO:6 is replaced with another amino acid, wherein said modified protox is tolerant to a herbicide in amounts that inhibit the naturally occurring protox activity. Particularly preferred is said DNA molecule wherein said cysteine is replaced with a phenylalanine or lysine, most preferred, wherein said cysteine is replaced with a phenylalanine.

Also preferred is a DNA encoding a modified protoporphyrinogen oxidase (protox) comprising a plant protox wherein the isoleucine occurring at the position corresponding to amino acid 419 of SEQ ID NO:6 is replaced with another amino acid, wherein said modified protox is tolerant to a herbicide in amounts that inhibit the naturally occurring protox activity. Particularly preferred is a DNA molecule, wherein said isoleucine is replaced with a threonine, histidine, glycine or asparagine most preferred, wherein said isoleucine is replaced with a threonine.

Also preferred is a DNA molecule encoding a modified protoporphyrinogen oxidase (protox) comprising a plant protox wherein the alanine occurring at the position corresponding to amino acid 164 of SEQ ID NO:6 is replaced with another amino acid, wherein said modified protox is tolerant to a herbicide in amounts that inhibit the naturally occurring protox activity. Particularly preferred is a DNA molecule wherein said alanine is replaced with a threonine, leucine or valine.

Also preferred is a DNA molecule encoding a modified protoporphyrinogen oxidase (protox) comprising a plant protox wherein the glycine occurring at the position corresponding to amino acid 165 of SEQ ID NO:6 is replaced with another amino acid, wherein said modified protox is tolerant to a herbicide in amounts that inhibit the naturally occurring protox activity. Particularly preferred is a DNA molecule wherein said glycine is replaced with a serine or leucine.

Also preferred is a DNA molecule encoding a modified protoporphyrinogen oxidase (protox) comprising a plant protox wherein the tyrosine occurring at the position corresponding to amino acid 370 of SEQ ID NO:6 is replaced with another amino acid, wherein said modified protox is tolerant to a herbicide in amounts that inhibit the naturally occurring protox activity. Particularly preferred is a DNA molecule wherein said tyrosine is replaced with a isoleucine or methionine.

Also preferred is a DNA molecule encoding a modified protoporphyrinogen oxidase (protox) comprising a plant protox wherein the valine occurring at the position corresponding to amino acid 356 of SEQ ID NO:10 is replaced with another amino acid, wherein said modified protox is tolerant to a herbicide in amounts that inhibit the naturally occurring protox activity. Particularly preferred is a DNA molecule wherein said valine is replaced with a leucine.

Also preferred is a DNA molecule encoding a modified protoporphyrinogen oxidase (protox) comprising a plant protox wherein the serine occurring at the position corresponding to amino acid 421 of SEQ ID NO:10 is replaced with another amino acid, wherein said modified protox is tolerant to a herbicide in amounts that inhibit the naturally occurring protox activity. Particularly preferred is a DNA molecule wherein said serine is replaced with a proline.

Also preferred is a DNA molecule encoding a modified protoporphyrinogen oxidase (protox) comprising a plant protox wherein the valine occurring at the position corresponding to amino acid 502 of SEQ ID NO:10 is replaced with another amino acid, wherein said modified protox is tolerant to a herbicide in amounts that inhibit the naturally occurring protox activity. Particularly preferred is a DNA molecule wherein said valine is replaced with a alanine.

Also preferred is a DNA molecule encoding a modified protoporphyrinogen oxidase (protox) comprising a plant protox wherein the alanine occurring at the position corresponding to amino acid 211 of SEQ ID NO:10 is replaced with another amino acid, wherein said modified protox is tolerant to a herbicide in amounts that inhibit the naturally occurring protox activity. Particularly preferred is a DNA molecule wherein said alanine is replaced with a valine or threonine.

Also preferred is a DNA molecule encoding a modified protoporphyrinogen oxidase (protox) comprising a plant protox wherein the glycine occurring at the position corresponding to amino acid 212 of SEQ ID NO:10 is replaced with another amino acid, wherein said modified protox is tolerant to a herbicide in amounts that inhibit the naturally occurring protox activity. Particularly preferred is a DNA molecule wherein said glycine is replaced with a serine.

Also preferred is a DNA encoding a modified protoporphyrinogen oxidase (protox) comprising a plant protox wherein the isoleucine occurring at the position corresponding to amino acid 466 of SEQ ID NO:10 is replaced with another amino acid, wherein said modified protox is tolerant to a herbicide in amounts that inhibit the naturally occurring protox activity. Particularly preferred is a DNA molecule wherein said isoleucine is replaced with a threonine.

Also preferred is a DNA molecule encoding a modified protoporphyrinogen oxidase (protox) comprising a plant protox wherein the proline occurring at the position corresponding to amino acid 369 of SEQ ID NO:12 is replaced with another amino acid, wherein said modified protox is tolerant to a herbicide in amounts that inhibit the naturally occurring protox activity. Particularly preferred is a DNA molecule wherein said proline is replaced with a serine or histidine.

Also preferred is a DNA molecule encoding a modified protoporphyrinogen oxidase (protox) comprising a plant protox wherein the alanine occurring at the position corresponding to amino acid 226 of SEQ ID NO:12 is replaced with another amino acid, wherein said modified protox is tolerant to a herbicide in amounts that inhibit the naturally occurring protox activity. Particularly preferred is a DNA molecule, wherein said alanine is replaced with a threonine or leucine.

Also preferred is a DNA molecule encoding a modified protoporphyrinogen oxidase (protox) comprising a plant protox wherein the valine occurring at the position corresponding to amino acid 517 of SEQ ID NO:12 is replaced with another amino acid, wherein said modified protox is tolerant to a herbicide in amounts that inhibit the naturally occurring protox activity. Particularly preferred is a DNA molecule wherein said valine is replaced with a alanine.

Also preferred is a DNA molecule encoding a modified protoporphyrinogen oxidase (protox) comprising a plant protox wherein the tyrosine occurring at the position corresponding to amino acid 432 of SEQ ID NO:12 is replaced with another amino acid, wherein said modified protox is tolerant to a herbicide in amounts that inhibit the naturally occurring protox activity. Particularly preferred is a DNA molecule wherein said tyrosine is replaced with a leucine or isoleucine.

Also preferred is a DNA molecule encoding a modified protoporphyrinogen oxidase (protox) comprising a plant protox wherein the proline occurring at the position corresponding to amino acid 365 of SEQ ID NO:16 is replaced with another amino acid, wherein said modified protox is tolerant to a herbicide in amounts that inhibit the naturally occurring protox activity. Particularly preferred is a DNA molecule wherein said proline is replaced with a serine.

Also preferred is a DNA molecule encoding a modified protoporphyrinogen oxidase (protox) comprising a plant protox wherein the tyrosine occurring at the position corresponding to amino acid 428 of SEQ ID NO:16 is replaced with another amino acid, wherein said modified protox is tolerant to a herbicide in amounts that inhibit the naturally occurring protox activity. Particularly preferred is a DNA molecule wherein said tyrosine is replaced with a cysteine or arginine.

Also preferred is a DNA encoding a modified protoporphyrinogen oxidase (protox) comprising a plant protox wherein the tyrosine occurring at the position corresponding to amino acid 449 of SEQ ID NO:18 is replaced with another amino acid, wherein said modified protox is tolerant to a herbicide in amounts that inhibit the naturally occurring protox activity. Particularly preferred is a DNA molecule wherein said tyrosine is replaced with a cysteine, leucine, isoleucine, valine or methionine.

The present invention is further directed to a DNA molecule encoding a modified protoporphyrinogen oxidase (protox) comprising a plant protox having a first amino acid substitution and a second amino acid substitution; said first amino acid substitution having the property of conferring resistance to a protox inhibitor; and said second amino acid substitution having the property of enhancing said resistance conferred by said first amino acid substitution. Preferred is a DNA molecule encoding a modified protoporphyrinogen oxidase (protox) comprising a plant protox, wherein said plant is selected from the group consisting of maize, wheat, soybean, cotton, sugar beet, rape, rice, sorghum, sugar cane, and Arabidopsis. More preferred is a DNA molecule encoding a modified protoporphyrinogen oxidase (protox) comprising a plant protox, wherein said plant is selected from the group consisting of maize, wheat, soybean, sugar beet, and Arabidopsis.

Preferred is a DNA molecule wherein said second amino acid substitution occurs at a position selected from the group consisting of:

(i) the position corresponding to the serine at amino acid 305 of SEQ ID NO:2;

(ii) the position corresponding to the threonine at amino acid 249 of SEQ ID NO:2;

(iii) the position corresponding to the proline at amino acid 118 of SEQ ID NO:2;

(iv) the position corresponding to the asparagine at amino acid 425 of SEQ ID NO:2; and

(v) the position corresponding to the tyrosine at amino acid 498 of SEQ ID NO:2.

Also preferred is a DNA molecule wherein said first amino acid substitution occurs at a position selected from the group consisting of:

(a) the position corresponding to the alanine at amino acid 164 of SEQ ID NO:6;

(b) the position corresponding to the glycine at amino acid 165 of SEQ ID NO:6;

(c) the position corresponding to the tyrosine at amino acid 370 of SEQ ID NO:6;

(d) the position corresponding to the cysteine at amino acid 159 of SEQ ID NO:6;

(e) the position corresponding to the isoleucine at amino acid 419 of SEQ ID NO:6.

(f) the position corresponding to the valine at amino acid 356 of SEQ ID NO:10;

(g) the position corresponding to the serine at amino acid 421 of SEQ ID NO:10;

(h) the position corresponding to the valine at amino acid 502 of SEQ ID NO:10;

(i) the position corresponding to the alanine at amino acid 211 of SEQ ID NO:10;

(k) the position corresponding to the glycine at amino acid 212 of SEQ ID NO:10;

(l) the position corresponding to the isoleucine at amino acid 466 of SEQ ID NO:10;

(m) the position corresponding to the proline at amino acid 369 of SEQ ID NO:12;

(n) the position corresponding to the alanine at amino acid 226 of SEQ ID NO:12;

(o) the position corresponding to the tyrosine at amino acid 432 of SEQ ID NO:12;

(p) the position corresponding to the valine at amino acid 517 of SEQ ID NO:12;

(q) the position corresponding to the tyrosine at amino acid 428 of SEQ ID NO:16;

(r) the position corresponding to the proline at amino acid 365 of SEQ ID NO:16; and

(s) the position corresponding to the tyrosine at amino acid 449 of SEQ ID NO:18.

Particularly preferred is a DNA molecule encoding a modified protoporphyrinogen oxidase (protox) comprising a plant protox wherein said plant protox comprises an amino acid sequence selected from the group consisting of SEQ ID NOs: 2, 4, 6, 8, 10, 12, 16, 18, 20, 22, and 37. Most preferred is a DNA molecule encoding a modified protoporphyrinogen oxidase (protox) comprising a plant protox, wherein said plant protox comprises an amino acid sequence selected from the group consisting of SEQ ID NOs: 2, 4, 6, 8, 10, 12, and 18.

More preferred is a DNA molecule, wherein said first amino acid substitution occurs at a position selected from the group consisting of

(a) the position corresponding to the alanine at amino acid 164 of SEQ ID NO:6;

(b) the position corresponding to the glycine at amino acid 165 of SEQ ID NO:6;

(c) the position corresponding to the tyrosine at amino acid 370 of SEQ ID NO:6;

(d) the position corresponding to the cysteine at amino acid 159 of SEQ ID NO:6;

(e) the position corresponding to the isoleucine at amino acid 419 of SEQ ID NO:6.

More preferred is a DNA molecule wherein said second amino acid substitution occurs at the position corresponding to the serine at amino acid 305 of SEQ ID NO:2 and said first amino acid substitution occurs at a position selected from the group consisting of

(a) the position corresponding to the alanine at amino acid 164 of SEQ ID NO:6;

(b) the position corresponding to the tyrosine at amino acid 370 of SEQ ID NO:6.

Particularly preferred is a DNA molecule wherein said serine occurring at the position corresponding to amino acid 305 of SEQ ID NO:2 is replaced with leucine.

More preferred is a DNA molecule wherein said second amino acid substitution occurs at the position corresponding to the threonine at amino acid 249 of SEQ ID NO:2 and said first amino acid substitution occurs at a position selected from the group consisting of

(a) the position corresponding to the alanine at amino acid 164 of SEQ ID NO:6; and

(b) the position corresponding to the tyrosine at amino acid 370 of SEQ ID NO:6.

Particularly preferred is a DNA wherein said threonine occurring at the position corresponding to amino acid 249 of SEQ ID NO:2 is replaced with an amino acid selected from the group consisting of isoleucine and alanine.

More preferred is a DNA molecule wherein said second amino acid substitution occurs at the position corresponding to the proline at amino acid 118 of SEQ ID NO:2 and said first amino acid substitution occurs at a position selected from the group consisting of

(a) the position corresponding to the alanine at amino acid 164 of SEQ ID NO:6; and

(b) the position corresponding to the tyrosine at amino acid 370 of SEQ ID NO:6.

Particularly preferred is a DNA molecule wherein said proline occurring at the position corresponding to amino acid 118 of SEQ ID NO:2 is replaced with a leucine.

More preferred is a DNA molecule wherein said second amino acid substitution occurs at the position corresponding to the asparagine at amino acid 425 of SEQ ID NO:2 and said first amino acid substitution occurs at a position selected from the group consisting of

(a) the position corresponding to the alanine at amino acid 164 of SEQ ID NO:6; and

(b) the position corresponding to the tyrosine at amino acid 370 of SEQ ID NO:6.

Particularly preferred is a DNA molecule wherein said asparagine occurring at the position corresponding to amino acid 425 of SEQ ID NO:2 is replaced with a serine.

More preferred is a DNA molecule wherein said second amino acid substitution occurs the position corresponding to the tyrosine at amino acid 498 of SEQ ID NO:2 and said first amino acid substitution occurs at a position selected from the group consisting of

(a) the position corresponding to the alanine at amino acid 164 of SEQ ID NO:6; and

(b) the position corresponding to the tyrosine at amino acid 370 of SEQ ID NO:6.

Particularly preferred is a DNA molecule wherein said tyrosine occurring at the position corresponding to amino acid 498 of SEQ ID NO:2 is replaced with a cysteine.

More preferred is a DNA molecule wherein said tyrosine occurring at the position corresponding to amino acid 370 of SEQ ID NO:6 is replaced with an amino acid selected from the group consisting of cysteine, isoleucine, leucine, threonine, valine and methionine.

Particularly preferred is a DNA molecule wherein said tyrosine occurring at the position corresponding to amino acid 370 of SEQ ID NO:6 is replaced with an amino acid selected from the group consisting of cysteine, isoleucine, leucine, threonine and methionine.

More preferred is a DNA molecule wherein said alanine occurring at the position corresponding to residue 164 of SEQ ID NO:6 is replaced with an amino acid selected from the group consisting of valine, threonine, leucine, cysteine and tyrosine.

More preferred is a DNA molecule wherein said glycine occurring at the position corresponding to residue 165 of SEQ ID NO:6 is replaced with an amino acid selected from the group consisting of serine and leucine.

Particularly preferred is a DNA molecule wherein said glycine occurring at the position corresponding to residue 165 of SEQ ID NO:6 is replaced with a serine.

More preferred is a DNA molecule wherein said cysteine occurring at the position corresponding to residue 159 of SEQ ID NO:6 is replaced with an amino acid selected from the group consisting of phenylalanine and lysine.

Particularly preferred is a DNA molecule wherein said cysteine occurring at the position corresponding to residue 159 of SEQ ID NO:6 is replaced with a phenylalanine.

More preferred is a DNA molecule wherein said isoleucine occurring at the position corresponding to residue 419 of SEQ ID NO:6 is replaced with an amino acid selected from the group consisting of threonine, histidine, glycine and asparagine.

Particularly preferred is a DNA molecule wherein said isoleucine occurring at the position corresponding to residue 419 of SEQ ID NO:6 is replaced with a threonine.

More preferred is a DNA molecule wherein said valine occurring at the position corresponding to residue 356 of SEQ ID NO:10 is replaced with a leucine.

More preferred is a DNA molecule wherein said serine occurring at the position corresponding to residue 421 of SEQ ID NO:10 is replaced with a proline.

More preferred is a DNA molecule wherein said valine occurring at the position corresponding to residue 502 of SEQ ID NO:10 is replaced with a alanine.

More preferred is a DNA molecule wherein said isoleucine occurring at the position corresponding to residue 466 of SEQ ID NO:10 is replaced with a threonine.

More preferred is a DNA molecule wherein said glycine occurring at the position corresponding to residue 212 of SEQ ID NO:10 is replaced with a serine.

More preferred is a DNA molecule wherein said alanine occurring at the position corresponding to residue 211 of SEQ ID NO:10 is replaced with a valine or threonine.

More preferred is a DNA molecule wherein said proline occurring at the position corresponding to residue 369 of SEQ ID NO:12 is replaced with a serine or a histidine.

More preferred is a DNA molecule wherein said alanine occurring at the position corresponding to residue 226 of SEQ ID NO:12 is replaced with a leucine or threonine.

More preferred is a DNA molecule wherein said tyrosine occurring at the position corresponding to residue 432 of SEQ ID NO:12 is replaced with a leucine or isoleucine.

More preferred is a DNA molecule wherein said valine occurring at the position corresponding to residue 517 of SEQ ID NO:12 is replaced with a alanine.

More preferred is a DNA molecule wherein said tyrosine occurring at the position corresponding to residue 428 of SEQ ID NO:16 is replaced with cysteine or arginine.

More preferred is a DNA molecule wherein said proline occurring at the position corresponding to residue 365 of SEQ ID NO:16 is replaced with serine.

More preferred is a DNA molecule wherein said proline occurring at the position corresponding to residue 449 of SEQ ID NO:18 is replaced with an amino acid selected from the group consisting of leucine, isoleucine, valine and methionine.

The present invention is directed to expression cassettes and recombinant vectors comprising said expression cassettes comprising essentially a promoter, but especially a promoter that is active in a plant, operatively linked to a DNA molecule encoding the protoporphyrinogen oxidase (protox) enzyme from a eukaryotic organism according to the invention. The expression cassette according to the invention may in addition further comprise a signal sequence operatively linked to said DNA molecule, wherein said signal sequence is capable of targeting the protein encoded by said DNA molecule into the chloroplast or the mitochondria.

The invention relates to a chimeric gene, which comprises an expression cassette comprising essentially a promoter, but especially a promoter that is active in a plant, operatively linked to a heterologous DNA molecule encoding a protoporphyrinogen oxidase (protox) enzyme from a eukaryotic organism according to the invention. Preferred is a chimeric gene, wherein the DNA molecule encodes an protoporphyrinogen oxidase (protox) enzyme from a plant selected from the group consisting of Arabidopsis, sugar cane, soybean, barley, cotton, tobacco, sugar beet, oilseed rape, maize, wheat, sorghum, rye, oats, turf and forage grasses, millet, forage and rice. More preferred is a chimeric gene, wherein the DNA molecule encodes an protoporphyrinogen oxidase (protox) enzyme from a plant selected from the group consisting of soybean, cotton, tobacco, sugar beet, oilseed rape, maize, wheat, sorghum, rye, oats, turf grass, and rice. Particularly preferred is a chimeric gene, wherein the DNA molecule encodes an protoporphyrinogen oxidase (protox) enzyme from a plant selected from the group consisting of wheat, soybean, cotton, sugar beet, rape, rice and sorghum. Most preferred is a chimeric gene, wherein the DNA molecule encodes an protoporphyrinogen oxidase (protox) enzyme from a plant selected from the group consisting of soybean, sugar beet, and wheat.

More preferred is a chimeric gene comprising a promoter active in a plant operatively linked to a heterologous DNA molecule encoding a protoporphyrinogen oxidase (protox) selected from the group consisting of a wheat protox comprising the sequence set forth in SEQ ID NO:10, a soybean protox comprising the sequence set forth in SEQ ID NO:12, cotton protox comprising the sequence set forth in SEQ ID NO:16, a sugar beet protox comprising the sequence set forth in SEQ ID NO:18, a rape protox comprising the sequence set forth in SEQ ID NO:20, a rice protox comprising the sequence set forth in SEQ ID NO:22, a sorghum protox comprising the sequence set forth in SEQ ID NO:24, and a sugar cane protox comprising the sequence set forth in SEQ ID NO:37. More preferred is a chimeric gene, wherein the protoporphyrinogen oxidase (protox) is selected from the group consisting of a wheat protox comprising the sequence set forth in SEQ ID NO:10, a soybean protox comprising the sequence set forth in SEQ ID NO:12, and a sugar beet protox comprising the sequence set forth in SEQ ID NO:18.

Particularly preferred is a chimeric gene, wherein the DNA molecule encodes a protein from an Arabidopsis species having protox-1 activity or protox-2 activity, preferably wherein said protein comprises the amino acid sequence set forth in SEQ ID NO:2 or SEQ ID NO:4.

Particularly preferred is a chimeric gene, wherein the DNA molecule encodes a protein from maize having protox-1 activity or protox-2 activity, preferably wherein said protein comprises the amino acid sequence set forth in set forth in SEQ ID NO:6 or SEQ ID NO:8.

Particularly preferred is a chimeric gene, wherein the DNA molecule encodes a protein from wheat having protox-1 activity preferably wherein said protein comprises the amino acid sequence set forth in SEQ ID NO:10.

Particularly preferred is a chimeric gene, wherein the DNA molecule encodes a protein from soybean having protox-1 activity, preferably wherein said protein comprises the amino acid sequence set forth in SEQ ID NO:12.

Particularly preferred is a chimeric gene, wherein the DNA molecule encodes a protein from cotton having protox-1 activity, preferably wherein said protein comprises the amino acid sequence set forth in SEQ ID NO:16.

Particularly preferred is a chimeric gene, wherein the DNA molecule encodes a protein from sugar beet having protox-1 activity, preferably wherein said protein comprises the amino acid sequence set forth in SEQ ID NO:18.

Particularly preferred is a chimeric gene, wherein the DNA molecule encodes a protein from rape having protox-1 activity, preferably wherein said protein comprises the amino acid sequence set forth in SEQ ID NO:20.

Particularly preferred is a chimeric gene, wherein the DNA molecule encodes a protein from rice having protox-1 activity, preferably wherein said protein comprises the amino acid sequence set forth in SEQ ID NO:22.

Particularly preferred is a chimeric gene, wherein the DNA molecule encodes a protein from sorghum having protox-1 activity, preferably wherein said protein comprises the amino acid sequence set forth in SEQ ID NO:24.

Particularly preferred is a chimeric gene, wherein the DNA molecule encodes a protein from sugar cane having protox-1 activity, preferably wherein said protein comprises the amino acid sequence set forth in SEQ ID NO:37.

The invention also embodies a chimeric gene, which comprises an expression cassette comprising essentially a promoter, but especially a promoter that is active in a plant, operatively linked to the DNA molecule encoding an protoporphyrinogen oxidase (protox) enzyme from a eukaryotic organism according to the invention, which is resistant to herbicides at levels that inhibit the corresponding unmodified version of the enzyme. Preferred is a chimeric gene, wherein the DNA molecule encodes an protoporphyrinogen oxidase (protox) enzyme from a plant selected from the group consisting of Arabidopsis, sugar cane, soybean, barley, cotton, tobacco, sugar beet, oilseed rape, maize, wheat, sorghum, rye, oats, turf and forage grasses, millet, forage and rice. More preferred is a chimeric gene, wherein the DNA molecule encodes an protoporphyrinogen oxidase (protox) enzyme from a plant selected from the group consisting of soybean, cotton, tobacco, sugar beet, oilseed rape, maize, wheat, sorghum, rye, oats, turf grass, and rice. Particularly preferred is a chimeric gene, wherein the DNA molecule encodes an protoporphyrinogen oxidase (protox) enzyme from a plant selected from the group consisting of Arabidopsis, soybean, cotton, sugar beet, oilseed rape, maize, wheat, sorghum, and rice.

Encompassed by the present invention is a chimeric gene comprising a promoter that is active in a plant operatively linked to the DNA molecule encoding a modified protoporphyrinogen oxidase (protox) comprising a eukaryotic protox having at least one amino acid modification, wherein said amino acid modification having the property of conferring resistance to a protox inhibitor.

Also encompassed by the present invention is a chimeric gene comprising a promoter that is active in a plant operatively linked to the DNA molecule encoding a modified protoporphyrinogen oxidase (protox) comprising a plant protox having a first amino acid substitution and a second amino acid substitution; said first amino acid substitution having the property of conferring resistance to a protox inhibitor; and said second amino acid substitution having the property of enhancing said resistance conferred by said first amino acid substitution. Preferred is said chimeric gene additionally comprising a signal sequence operatively linked to said DNA molecule, wherein said signal sequence is capable of targeting the protein encoded by said DNA molecule into the chloroplast or in the mitochondria.

The chimeric gene according to the invention may in addition further comprise a signal sequence operatively linked to said DNA molecule, wherein said signal sequence is capable of targeting the protein encoded by said DNA molecule into the chloroplast. The chimeric gene according to the invention may in addition further comprise a signal sequence operatively linked to said DNA molecule, wherein said signal sequence is capable of targeting the protein encoded by said DNA molecule into the mitochondria.

Also encompassed by the present invention is any of the DNA sequences mentioned herein before, which is stably integrated into a host genome.

The invention further relates to a recombinant DNA molecule comprising a plant protoporphyrinogen oxidase (protox) or a functionally equivalent derivative thereof.

The invention further relates to a recombinant DNA vector comprising said recombinant DNA molecule

A further object of the invention is a recombinant vector comprising the chimeric gene according to the invention, wherein said vector is capable of being stably transformed into a host cell.

A further object of the invention is a recombinant vector comprising the chimeric gene according to the invention, wherein said vector is capable of being stably transformed into a plant, plant seeds, plant tissue or plant cell. Preferred is a recombinant vector comprising the chimeric gene according to the invention, wherein said vector is capable of being stably transformed into a plant. The plant, plant seeds, plant tissue or plant cell stably transformed with the vector is capable of expressing the DNA molecule encoding a protoporphyrinogen oxidase (protox). Preferred is a recombinant vector, wherein the plant, plant seeds, plant tissue or plant cell stably transformed with the said vector is capable of expressing the DNA molecule encoding a protoporphyrinogen oxidase (protox) from a plant that is resistant to herbicides at levels that inhibit the corresponding unmodified version of the enzyme.

Preferred is a recombinant vector comprising the chimeric gene comprising a promoter active in a plant operatively linked to a heterologous DNA molecule encoding a protoporphyrinogen oxidase (protox) selected from the group consisting of a wheat protox comprising the sequence set forth in SEQ ID NO:10, a soybean protox comprising the sequence set forth in SEQ ID NO:12, cotton protox comprising the sequence set forth in SEQ ID NO:16, a sugar beet protox comprising the sequence set forth in SEQ ID NO:18, a rape protox comprising the sequence set forth in SEQ ID NO:20, a rice protox comprising the sequence set forth in SEQ ID NO:22, a sorghum protox comprising the sequence set forth in SEQ ID NO:24, and a sugar cane protox comprising the sequence set forth in SEQ ID NO:37, wherein said vector is capable of being stably transformed into a host cell.

Also preferred is recombinant vector comprising the chimeric gene comprising a promoter that is active in a plant operatively linked to the DNA molecule encoding a modified protoporphyrinogen oxidase (protox) comprising a plant protox having a first amino acid substitution and a second amino acid substitution; said first amino acid substitution having the property of conferring resistance to a protox inhibitor; and said second amino acid substitution having the property of enhancing said resistance conferred by said first amino acid substitution, wherein said vector is capable of being stably transformed into a plant cell.

Also encompassed by the present invention is a host cell stably transformed with the vector according to the invention, wherein said host cell is capable of expressing said DNA molecule. Preferred is a host cell selected from the group consisting of a plant cell, a bacterial cell, a yeast cell, and an insect cell.

The present invention is further directed to plants and the progeny thereof, plant tissue and plant seeds tolerant to herbicides that inhibit the naturally occurring protox activity in these plants, wherein the tolerance is conferred by a gene expressing a modified inhibitor-resistant protox enzyme as taught herein. Representative plants include any plants to which these herbicides may be applied for their normally intended purpose. Preferred are agronomically important crops, i.e., angiosperms and gymnosperms such as Arabidopsis, sugar cane, soybean, barley, cotton, tobacco, sugar beet, oilseed rape, maize, wheat, sorghum, rye, oats, tomato, potato, turf and forage grasses, millet, forage, and rice and the like. More preferred are agronomically important crops, i.e., angiosperms and gymnosperms such as Arabidopsis, cotton, soybean, rape, sugar beet, maize, rice, wheat, barley, oats, rye, sorghum, millet, turf, forage, turf grasses. Particularly preferred are agronomically important crops, i.e., angiosperms and gymnosperms such as Arabidopsis, soybean, cotton, sugar beet, oilseed rape, maize, wheat, sorghum, and rice.

Preferred is a plant comprising the DNA molecule encoding a modified protoporphyrinogen oxidase (protox) comprising a plant protox having a first amino acid substitution and a second amino acid substitution; said first amino acid substitution having the property of conferring resistance to a protox inhibitor; and said second amino acid substitution having the property of enhancing said resistance conferred by said first amino acid substitution, wherein said DNA molecule is expressed in said plant and confers upon said plant tolerance to a herbicide in amounts that inhibit naturally occurring protox activity. Preferred is a plant, wherein said DNA molecule replaces a corresponding naturally occurring protox coding sequence. Comprised by the present invention is a plant and the progeny thereof comprising the chimeric gene according to the invention, wherein said chimeric gene confers upon said plant tolerance to a herbicide in amounts that inhibit naturally occurring protox activity.

Encompassed by the present invention are transgenic plant tissue, including plants and the progeny thereof, seeds, and cultured tissue, stably transformed with at least one chimeric gene according to the invention. Preferred is transgenic plant tissue, including plants, seeds, and cultured tissue, stably transformed with at least one chimeric gene that comprises an expression cassette comprising essentially a promoter, but especially a promoter that is active in a plant, operatively linked to the DNA molecule encoding an protoporphyrinogen oxidase (protox) enzyme that is resistant to herbicides at levels that inhibit the corresponding unmodified version of the enzyme in the plant tissue.

The present invention is further directed to plants, plant tissue, plant seeds, and plant cells tolerant to herbicides that inhibit the naturally occurring protox activity in these plants, wherein the tolerance is conferred by increasing expression of wild-type herbicide-sensitive protox. This results in a level of a protox enzyme in the plant cell at least sufficient to overcome growth inhibition caused by the herbicide. The level of expressed enzyme generally is at least two times, preferably at least five times, and more preferably at least ten times the natively expressed amount. Increased expression may be due to multiple copies of a wild-type protox gene; multiple occurrences of the coding sequence within the gene (i.e. gene amplification) or a mutation in the non-coding, regulatory sequence of the endogenous gene in the plant cell. Plants having such altered gene activity can be obtained by direct selection in plants by methods known in the art (see, e.g. U.S. Pat. No. 5,162,602, and U.S. Pat. No. 4,761,373, and references cited therein). These plants also may be obtained by genetic engineering techniques known in the art. Increased expression of a herbicide-sensitive protox gene can also be accomplished by stably transforming a plant cell with a recombinant or chimeric DNA molecule comprising a promoter capable of driving expression of an associated structural gene in a plant cell operatively linked to a homologous or heterologous structural gene encoding the protox enzyme.

The recombinant DNA molecules of the invention can be introduced into the plant cell in a number of art-recognized ways. Those skilled in the art will appreciate that the choice of method might depend on the type of plant, i.e. monocot or dicot, targeted for transformation. Suitable methods of transforming plant cells include microinjection (Crossway et al., BioTechniques 4.320-334 (1986)), electroporation (Riggs et al, Proc. Natl. Acad. Sci. USA 83:5602-5606 (1986), Agrobacterium mediated transformation (Hinchee et al., Biotechnology 6:915-921 (1988)), direct gene transfer (Paszkowski et al., EMBO J. 3.2717-2722 (1984)), ballistic particle acceleration using devices available from Agracetus, Inc., Madison, Wis. and Dupont, Inc., Wilmington, Del. (see, for example, Sanford et al., U.S. Pat. No. 4,945,050; and McCabe et al., Biotechnology 6.923-926 (1988)), protoplast Transformation/regeneration methods (see U.S. Pat. No. 5,350,689 issued Sept. 27, 1994 to Ciba-Geigy Corp.), and pollen transformation (see U.S. Pat. No. 5,629,183). Also see, Weissinger et al., Annual Rev. Genet. 22:421-477 (1988); Sanford et al., Particulate Science and Technology 5.27-37 (1987)(onion); Christou et al., Plant Physiol. 87:671-674 (1988)(soybean); McCabe et al., Bio/Technology 6:923-926 (1988)(soybean); Datta et al., Bio/Technology 8:736-740 (1990)(rice); Klein et al., Proc. Natl. Acad. Sci. USA, 85:4305-4309 (1988)(maize); Klein et al., Bio/Technology 6:559-563 (1988)(maize); Klein et al., Plant Physiol. 91:440-444 (1988)(maize); Fromm et al., Bio/Technology 8:833-839 (1990); Gordon-Kamm et al., Plant Cell 2:603-618 (1990) (maize); and U.S. Pat. Nos. 5,591,616 and 5,679,558 (rice).

Comprised within the scope of the present invention are transgenic plants, in particular transgenic fertile plants transformed by means of the aforedescribed processes and their asexual and/or sexual progeny, which still are resistant or at least tolerant to inhibition by a herbicide at levels that normally are inhibitory to the naturally occurring protox activity in the plant. Progeny plants also include plants with a different genetic background than the parent plant, which plants result from a backcrossing program and still comprise in their genome the herbicide resistance trait according to the invention. Very especially preferred are hybrid plants that are resistant or at least tolerant to inhibition by a herbicide at levels that normally are inhibitory to the naturally occurring protox activity in the plant.

The transgenic plant according to the invention may be a dicotyledonous or a monocotyledonous plant. Preferred are monocotyledonous plants of the Graminaceae family involving Lolium, Zea, Triticum, Triticale, Sorghum, Saccharum, Bromus, Oryzae, Avena, Hordeum, Secale and Setaria plants. More preferred are transgenic maize, wheat, barley, sorghum, rye, oats, sugar cane, turf and forage grasses, millet and rice. Especially preferred are maize, wheat, sorghum, rye, oats, turf grasses and rice.

Among the dicotyledonous plants Arabidopsis, soybean, cotton, sugar beet, oilseed rape, tobacco, tomato, potato, and sunflower are more preferred herein. Especially preferred are soybean, cotton, tobacco, sugar beet, tomato, potato, and oilseed rape.

The expression ‘progeny’ is understood to embrace both, “asexually” and “sexually” generated progeny of transgenic plants. This definition is also meant to include all mutants and variants obtainable by means of known processes, such as for example cell fusion or mutant selection and that still exhibit the characteristic properties of the initial transformed plant, together with all crossing and fusion products of the transformed plant material. This also includes progeny plants that result from a backcrossing program, as long as the said progeny plants still contain the herbicide resistant trait according to the invention.

Another object of the invention concerns the proliferation material of transgenic plants. propagated sexually or asexually in vivo or in vitro. Particularly preferred within the scope of the present invention are protoplasts, cells, calli, tissues, organs, seeds, embryos, pollen, egg cells, zygotes, together with any other propagating material obtained from transgenic plants.

Parts of plants, such as for example flowers, stems, fruits, leaves, roots originating in transgenic plants or their progeny previously transformed by means of the process of the invention and therefore consisting at least in part of transgenic cells, are also an object of the present invention.

A further object of the invention is a method of producing plants, protoplasts, cells, calli, tissues, organs, seeds, embryos, pollen, egg cells, zygotes, together with any other propagating material, parts of plants, such as for example flowers, stems, fruits, leaves, roots originating in transgenic plants or their progeny previously transformed by means of the process of the invention, which therefore produce an inhibitor resistant form of a plant protox enzyme by transforming the plant, plant parts with the DNA according to the invention. Preferred is a method of producing a host cell comprising an isolated DNA molecule encoding a protein from a eukaryote having protoporphyrinogen oxidase (protox) activity comprising transforming the said host cell with a recombinant vector molecule according to the invention. Further preferred is a method of producing a plant cell comprising an isolated DNA molecule encoding a protein from a eukaryote having protoporphyrinogen oxidase (protox) activity comprising transforming the said plant cell with a recombinant vector molecule according to the invention. Preferred is a method of producing transgenic progeny of a transgenic parent plant comprising an isolated DNA molecule encoding a protein from a eukaryote having protoporphyrinogen oxidase (protox) activity comprising transforming the said parent plant with a recombinant vector molecule according to the invention and transferring the herbicide tolerant trait to the progeny of the said transgenic parent plant involving known plant breeding techniques.

Preferred is a method for the production of plants, plant tissues, plant seeds and plant parts, which produce an inhibitor-resistant form of the plant protox enzyme, wherein the plants, plant tissues, plant seeds and plant parts have been stably transformed with a structural gene encoding the resistant protox enzyme. Particularly preferred is a method for the production of plants, plant tissues, plant seeds and plant parts, wherein the plants, plant tissues, plant seeds and plant parts have been stably transformed with the DNA according to the invention. Especially preferred is a method for the production of said plants, plant tissues, plant seeds and plant parts, which produce an inhibitor-resistant form of the plant protox enzyme, wherein the plants, plant tissues, plant seeds and plant parts have been prepared by direct selection techniques whereby herbicide resistant lines are isolated, characterized and developed.

The genetic properties engineered into the transgenic seeds and plants described above are passed on by sexual reproduction or vegetative growth and can thus be maintained and propagated in progeny plants. Generally said maintenance and propagation make use of known agricultural methods developed to fit specific purposes such as tilling, sowing or harvesting. Specialized processes such as hydroponics or greenhouse technologies can also be applied. As the growing crop is vulnerable to attack and damages caused by insects or infections as well as to competition by weed plants, measures are undertaken to control weeds, plant diseases, insects, nematodes, and other adverse conditions to improve yield. These include mechanical measures such a tillage of the soil or removal of weeds and infected plants, as well as the application of agrochemicals such as herbicides, fungicides, gametocides, nematicides, growth regulants, ripening agents and insecticides.

Use of the advantageous genetic properties of the transgenic plants and seeds according to the invention can further be made in plant breeding that aims at the development of plants with improved properties such as tolerance of pests, herbicide tolerance, or stress tolerance, improved nutritional value, increased yield, or improved structure causing less loss from lodging or shattering. The various breeding steps are characterized by well-defined human intervention such as selecting the lines to be crossed, directing pollination of the parental lines, or selecting appropriate progeny plants. Depending on the desired properties different breeding measures are taken. The relevant techniques are well known in the art and include but are not limited to hybridization, inbreeding, backcross breeding, multiline breeding, variety blend, interspecific hybridization, aneuploid techniques, etc. Hybridization techniques also include the sterilization of plants to yield male or female sterile plants by mechanical, chemical or biochemical means. Cross pollination of a male sterile plant with pollen of a different line assures that the genome of the male sterile but female fertile plant will uniformly obtain properties of both parental lines. Thus, the transgenic seeds and plants according to the invention can be used for the breeding of improved plant lines that for example increase the effectiveness of conventional methods such as herbicide or pesticide treatment or allow to dispense with said methods due to their modified genetic properties. Alternatively new crops with improved stress tolerance can be obtained that, due to their optimized genetic “equipment”, yield harvested product of better quality than products that were not able to tolerate comparable adverse developmental conditions.

In seeds production germination quality and uniformity of seeds are essential product characteristics, whereas germination quality and uniformity of seeds harvested and sold by the farmer is not important. As it is difficult to keep a crop free from other crop and weed seeds, to control seedborne diseases, and to produce seed with good germination, fairly extensive and well-defined seed production practices have been developed by seed producers, who are experienced in the art of growing, conditioning and marketing of pure seed. Thus, it is common practice for the farmer to buy certified seed meeting specific quality standards instead of using seed harvested from his own crop. Propagation material to be used as seeds is customarily treated with a protectant coating comprising herbicides, insecticides, fungicides, bactericides, nematicides, molluscicides or mixtures thereof. Customarily used protectant coatings comprise compounds such as captan, carboxin, thiram (TMTD®), methalaxyl (Apron®), and pirimiphos-methyl (Actellic®). If desired these compounds are formulated together with further carriers, surfactants or application-promoting adjuvants customarily employed in the art of formulation to provide protection against damage caused by bacterial, fungal or animal pests. The protectant coatings may be applied by impregnating propagation material with a liquid formulation or by coating with a combined wet or dry formulation. Other methods of application are also possible such as treatment directed at the buds or the fruit.

It is thus a further object of the present invention to provide plant propagation material for cultivated plants, but especially plant seed that is treated with an seed protectant coating customarily used in seed treatment.

It is a further aspect of the present invention to provide new agricultural methods such as the methods exemplified above, which are characterized by the use of transgenic plants, transgenic plant material, or transgenic seed according to the present invention. Comprised by the present invention is an agricultural method, wherein a transgenic plant or the progeny thereof is used comprising a chimeric gene according to the invention in an amount sufficient to express herbicide resistant forms of herbicide target proteins in a plant to confer tolerance to the herbicide.

To breed progeny from plants transformed according to the method of the present invention, a method such as that which follows may be used: maize plants produced as described in the examples set forth below are grown in pots in a greenhouse or in soil, as is known in the art, and permitted to flower. Pollen is obtained from the mature tassel and used to pollinate the ears of the same plant, sibling plants, or any desirable maize plant. Similarly, the ear developing on the transformed plant may be pollinated by pollen obtained from the same plant, sibling plants, or any desirable maize plant. Transformed progeny obtained by this method may be distinguished from non-transformed progeny by the presence of the introduced gene(s) and/or accompanying DNA (genotype), or the phenotype conferred. The transformed progeny may similarly be selfed or crossed to other plants, as is normally done with any plant carrying a desirable trait. Similarly, tobacco or other transformed plants produced by this method may be selfed or crossed as is known in the art in order to produce progeny with desired characteristics. Similarly, other transgenic organisms produced by a combination of the methods known in the art and this invention may be bred as is known in the art in order to produce progeny with desired characteristics.

The modified inhibitor-resistant protox enzymes of the invention have at least one amino acid substitution, addition or deletion relative to their naturally occurring counterpart (i.e. inhibitor-sensitive forms that occur naturally in a plant without being manipulated, either directly via recombinant DNA methodology or indirectly via selective breeding, etc., by man). Amino acid positions that may be modified to yield an inhibitor-resistant form of the protox enzyme, or enhance inhibitor resistance, are indicated in bold type in Table 1 in the context of plant protox-1 sequences from Arabidopsis, maize, soybean, cotton, sugar beet, rape, rice, sorghum and wheat. The skilled artisan will appreciate that equivalent changes may be made to any plant protox gene having a structure sufficiently similar to the protox enzyme sequences shown herein to allow alignment and identification of those amino acids that are modified according to the invention to generate inhibitor-resistant forms of the enzyme. Such additional plant protox genes may be obtained using standard techniques as described in International application No. PCT/IB95/00452 filed Jun. 8, 1995, published Dec. 21, 1995 as WO 95/34659 whose relevant parts are herein incorporated by reference.

DNA molecules encoding the herbicide resistant protox coding sequences taught herein may be genetically engineered for optimal expression in a crop plant. This may include altering the coding sequence of the resistance allele for optimal expression in the crop species of interest. Methods for modifying coding sequences to achieve optimal expression in a particular crop species are well known (see, e.g. Perlak et al., Proc. Natl. Acad. Sci. USA 88: 3324 (1991); Koziel et al., Bio/technol. 11: 194 (1993)).

Genetically engineering a protox coding sequence for optimal expression may also include operatively linking the appropriate regulatory sequences (i.e. promoter, signal sequence, transcriptional terminators). Examples of promoters capable of functioning in plants or plant cells (i.e., those capable of driving expression of the associated structural genes such as protox in plant cells) include the cauliflower mosaic virus (CaMV) 19S or 35S promoters and CaMV double promoters; nopaline synthase promoters; pathogenesis-related (PR) protein promoters; small subunit of ribulose bisphosphate carboxylase (ssuRUBISCO) promoters, heat shock protein promoter from Brassica with reference to EPA 0 559 603 (hsp80 promoter), Arabidopsis actin promoter and the SuperMas promoter with reference to WO 95/14098 and the like. Preferred promoters will be those that confer high level constitutive expression or, more preferably, those that confer specific high level expression in the tissues susceptible to damage by the herbicide. Preferred promoters are the rice actin promoter (McElroy et al., Mol. Gen. Genet. 231: 150 (1991)), maize ubiquitin promoter (EP 0 342 926; Taylor et al., Plant Cell Rep. 12: 491 (1993)), and the PR-1 promoter from tobacco, Arabidopsis, or maize (see U.S. Pat. No. 5,614,395 to Ryals et al., incorporated by reference herein in its entirety). The promoters themselves may be modified to manipulate promoter strength to increase protox expression, in accordance with art-recognized procedures.

The inventors have also discovered that another preferred promoter for use with the inhibitor-resistant protox coding sequences is the promoter associated with the native protox gene (i.e. the protox promoter; see copending, co-owned U.S. patent application Ser. No. 08/808,323, entitled “Promoters from Protoporphyrinogen Oxidase Genes”, incorporated by reference herein in its entirety.) The promoter sequence from an Arabidopsis protox-1 gene is set forth in SEQ ID NO:13, the promoter sequence from a maize protox-1 gene is set forth in SEQ ID NO:14, and the promoter sequence from a sugar beet protox-1 gene is set forth in SEQ ID NO:26.

Since the protox promoter itself is suitable for expression of inhibitor-resistant protox coding sequences, the modifications taught herein may be made directly on the native protox gene present in the plant cell genome without the need to construct a chimeric gene with heterologous regulatory sequences. Such modifications can be made via directed mutagenesis techniques such as homologous recombination and selected for based on the resulting herbicide-resistance phenotype (see, e.g. Example 10, Pazkowski et al., EMBO J. 7: 4021-4026 (1988), and U.S. Pat. No. 5,487,992, particularly columns 18-19 and Example 8). An added advantage of this approach is that besides containing the native protox promoter, the resulting modified gene will also include any other regulatory elements, such as signal or transit peptide coding sequences, which are part of the native gene.

In the event of transformation of the nuclear genome, signal or transit peptides may be fused to the protox coding sequence in chimeric DNA constructs of the invention to direct transport of the expressed protox enzyme to the desired site of action. Examples of signal peptides include those natively linked to the plant pathogenesis-related proteins, e.g. PR-1, PR-2, and the like. See, e.g., Payne et al., Plant Mol. Biol. 11:89-94 (1988). Examples of transit peptides include the chloroplast transit peptides such as those described in Von Heijne et al., Plant Mol. Biol. Rep. 9:104-126 (1991); Mazur et al., Plant Physiol. 85: 1110 (1987); Vorst et al., Gene 65: 59 (1988), and mitochondrial transit peptides such as those described in Boutry et al., Nature 328.340-342 (1987). Chloroplast and mitochondrial transit peptides are contemplated to be particularly useful with the present invention as protox enzymatic activity typically occurs within the mitochondria and chloroplast. Most preferred for use are chloroplast transit peptides, as inhibition of the protox enzymatic activity in the chloroplasts is contemplated to be the primary basis for the action of protox-inhibiting herbicides (Witkowski and Hailing, Plant Physiol. 87: 632 (1988); Lehnen et al., Pestic. Biochem. Physiol. 37: 239 (1990); Duke et al., Weed Sci. 39: 465 (1991)). Also included are sequences that result in localization of the encoded protein to various cellular compartments such as the vacuole. See, for example, Neuhaus et al., Proc. Natl. Acad. Sci. USA 88: 10362-10366 (1991) and Chrispeels, Ann. Rev. Plant Physiol. Plant Mol. Biol. 42: 21-53 (1991). The relevant disclosures of these publications are incorporated herein by reference in their entirety.

Chimeric genes of the invention may contain multiple copies of a promoter or multiple copies of the protox structural genes. In addition, the construct(s) may include coding sequences for markers and coding sequences for other peptides such as signal or transit peptides, each in proper reading frame with the other functional elements in the DNA molecule. The preparation of such constructs are within the ordinary level of skill in the art.

Useful markers include peptides providing herbicide, antibiotic or drug resistance, such as, for example, resistance to hygromycin, kanamycin, G418, gentamycin, lincomycin, methotrexate, glyphosate, phosphinothricin, or the like. These markers can be used to select cells transformed with the chimeric DNA constructs of the invention from untransformed cells. Other useful markers are peptidic enzymes that can be easily detected by a visible reaction, for example a color reaction, for example luciferase, β-glucuronidase, or β-galactosidase.

The method of positive selection of genetically transformed cells into which a desired nucleotide sequence can be incorporated by providing the transformed cells with a selective advantage is herein incorporated by reference as WO 94/20627.

Where a herbicide resistant protox allele is obtained via directed mutation of the native gene in a crop plant or plant cell culture from which a crop plant can be regenerated, it may be moved into commercial varieties using traditional breeding techniques to develop a herbicide tolerant crop without the need for genetically engineering the modified coding sequence and transforming it into the plant. Alternatively, the herbicide resistant gene may be isolated, genetically engineered for optimal expression and then transformed into the desired variety.

Genes encoding altered protox resistant to a protox inhibitor can also be used as selectable markers in plant cell transformation methods. For example, plants, plant tissue or plant cells transformed with a transgene can also be transformed with a gene encoding an altered protox capable of being expressed by the plant. The thus-transformed cells are transferred to medium containing the protox inhibitor wherein only the transformed cells will survive. Protox inhibitors contemplated to be particularly useful as selective agents are the diphenylethers {e.g. acifluorfen, 5-[2-chloro-4-(trifluoromethyl)phenoxy]-2-nitrobezoic acid; its methyl ester; or oxyfluorfen, 2-chloro-1-(3-ethoxy-4-nitrophenoxy)-4-(trifluorobenzene)}, oxidiazoles, (e.g. oxidiazon, 3-[2,4-dichloro-5-(1-methylethoxy)phenyl]-5-(1,1-dimethylethyl)-1,3,4-oxadiazol-2-(3H)-one), cyclic imides (e.g. S-23142, N-(4-chloro-2-fluoro-5-propargyloxyphenyl)-3,4,5,6-tetrahydrophthalimide; chlorophthalim, N-(4-chlorophenyl)-3,4,5,6-tetrahydrophthalimide), phenyl pyrazoles (e.g. TNPP-ethyl, ethyl 2-[1-(2,3,4-trichlorophenyl)-4-nitropyrazolyl-5-oxy]propionate; M&B 39279), pyridine derivatives (e.g. LS 82-556), and phenopylate and its O-phenylpyrrolidino- and piperidinocarbamate analogs and bicyclic Triazolones as disclosed in the International patent application WO 92/04827; EP 532146).

The method is applicable to any plant cell capable of being transformed with an altered protox-encoding gene, and can be used with any transgene of interest. Expression of the transgene and the protox gene can be driven by the same promoter functional on plant cells, or by separate promoters.

Modified inhibitor-resistant protox enzymes of the present invention are resistant to herbicides that inhibit the naturally occurring protox activity. The herbicides that inhibit protox include many different structural classes of molecules (Duke et al., Weed Sci. 39: 465 (1991); Nandihalli et al., Pesticide Biochem. Physiol. 43: 193 (1992); Matringe et al., FEBS Lett. 245: 35 (1989); Yanase and Andoh, Pesticide Biochem. Physiol. 35: 70 (1989)), including the diphenylethers (e.g. acifluorifen, 5-[2-chloro-4-(trifluoromethyl)phenoxy]-2-nitrobezoic acid; its methyl ester; or oxyfluorfen, 2-chloro-1-(3-ethoxy-4-nitrophenoxy)-4-(trifluorobenzene)), oxidiazoles (e.g. oxidiazon, 3-[2,4-dichloro-5-(1-methylethoxy)phenyl]-5-(1,1-dimethylethyl)-1,3,4-oxadiazol-2-(3J-one), cyclic imides (e.g. S-23142, N-(4-chloro-2-fluoro-5-propargyloxyphenyl)-3,4,5,6-tetrahydrophthalimide; chlorophthalim, N-(4-chlorophenyl)-3,4,5,6-tetrahydrophthalimide), phenyl pyrazoles (e.g. TNPP-ethyl, ethyl 2-[l -(2,3,4-trichlorophenyl)-4-nitropyrazolyl-5-oxy]propionate; M&B 39279), pyridine derivatives (e.g. LS 82-556), and phenopylate and its O-phenylpyrrolidino- and piperidinocarbamate analogs.

The diphenylethers of particular significance are those having the general formula

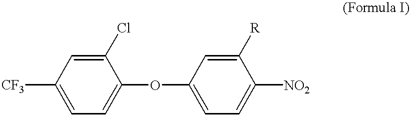

wherein R equals —COONa (Formula II), —CONHSO₂CH₃ (Formula III) or —COOCH₂COOC₂H₅ (Formula IV; see Maigrot et al., Brighton Crop Protection Conference-Weeds: 47-51 (1989)).

Additional diphenylethers of interest are those where R equals:

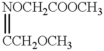

(Formula IVa; see Hayashi et al., Brighton Crop Protection Conference-Weeds: 53-58 (1989)).

An additional diphenylether of interest is one having the formula:

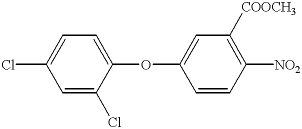

(Formula IVb; bifenox, see Dest et al., Proc. Northeast Weed Sci. Conf. 27:,31 (1973)).

A further diphenylether of interest is one having the formula:

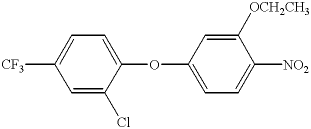

(Formula IVc; oxyfluorfen; see Yih and Swithenbank, J. Agric. Food Chem., 23: 592 (1975))

Yet another diphenylether of interest is one having the formula:

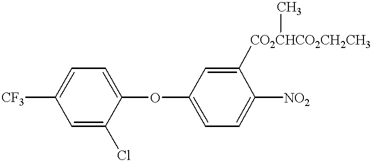

(Formula IVd; lactofen, see page 623 of “The Pesticide Manual”, 10^{th} ed., ed. by C. Tomlin, British Crop Protection Council, Surrey (1994))

Also of significance are the class of herbicides known as imides, having the general formula

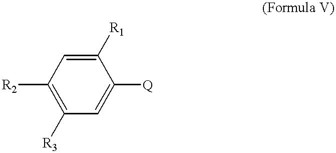

wherein Q equals

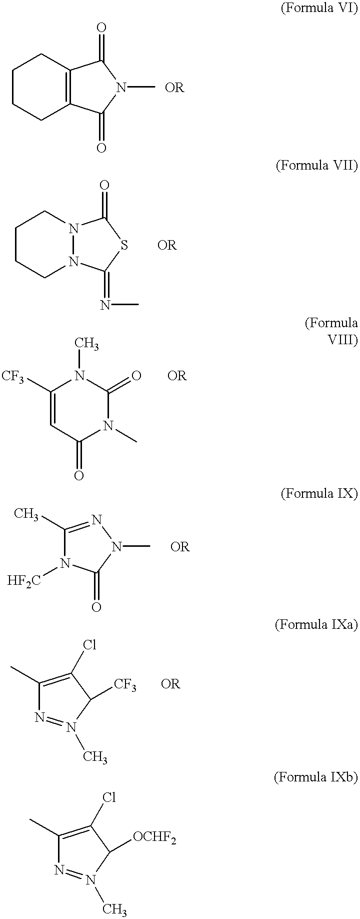

(see Hemper et al. (1995) in “Proceedings of the Eighth International Congress of Pesticide Chemistry”, Ragdale et al., eds., Amer. Chem. Soc, Washington, D.C., pp.42-48 (1994)); and R₁ equals H, Cl or F. R₂ equals Cl and R₃ is an optimally substituted ether, thioether, ester, amino or alkyl group. Alternatively, R2 and R₃ together may form a 5 or 6 membered heterocyclic ring. Examples of imide herbicides of particular interest are

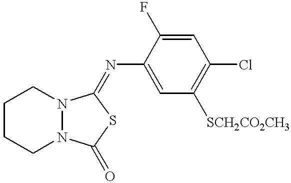

(Formula VIIa; fluthiacet-methyl, see Miyazawa et al., Brighton Crop Protection Conference-Weeds, pp.23-28 (1993))

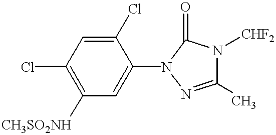

(Formula X sulfentrazone, see Van Saun et al., Brighton Crop Protection Conference-Weeds, pp. 77-82 (1991)).

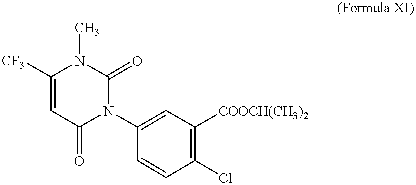

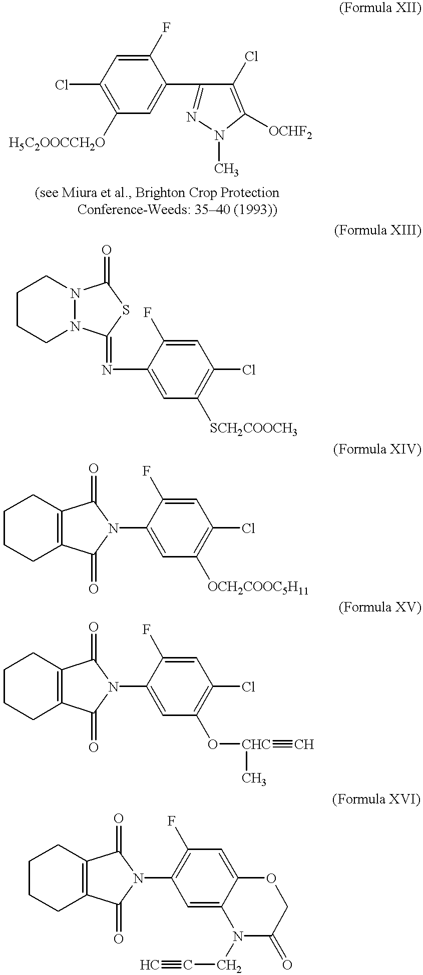

The herbicidal activity of the above compounds is described in the Proceedings of the 1991 Brighton Crop Protection Conference, Weeds (British Crop Protection Council) (Formulae X and XVI), Proceedings of the 1993 Brighton Crop Protection Conference, Weeds (British Crop Protection Council) (Formulae XII and XIII), U.S. Pat. No. 4,746,352 (Formula XI) and Abstracts of the Weed Science Society of America vol. 33, pg. 9 (1993)(Formula XIV).

The most preferred imide herbicides are those classified as aryluracils and having the general formula

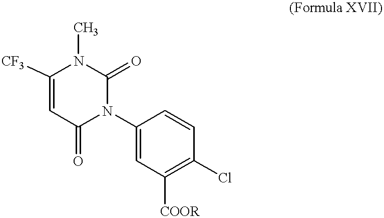

wherein R signifies the group (C₂₋₆-alkenyloxy)carbonyl-C₁₋₄-alkyl, as disclosed in U.S. Pat. No. 5,183,492, herein incorporated by reference.

Also of significance are herbicides having the general formula:

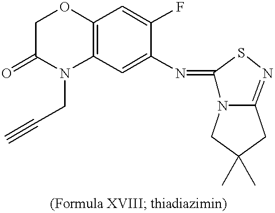

(see Weiler et al., Brighton Crop Protection Conference-Weeds, pp. 29-34 (1993));

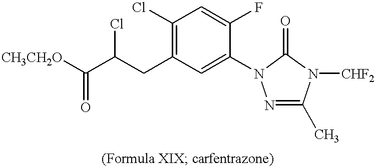

(see Van Saun et al., Brighton Crop Protection Conference-Weeds: pp. 19-22 (1993));

N-substituted pyrazoles of the general formula:

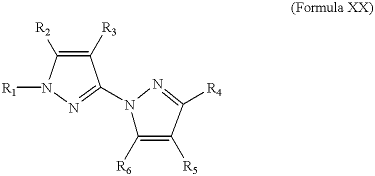

wherein R₁ is C₁-C₄-alkyl, optionally substituted by one or more halogen atoms;

R₂ is hydrogen, or a C₁-C₄-alkoxy, each of which is optionally substituted by one or more halogen atoms, or

R₁ and R₂ together from the group —(CH₂),-X—, where X is bound at R₂;

R₃ is hydrogen or halogen,

R₄ is hydrogen or C₁-C₄-alkyl,

R₅ is hydrogen, nitro, cyano or the group —COOR₆ or —CONR₇R₈, and

R₆ is hydrogen, C₁-C6-alkyl, C₂-C₆-alkenyl or C₂-C₆-alkynyl;

(see international patent publications WO 94/08999, WO 93/10100, and U.S. Pat. No. 5,405,829 assigned to Schering);

N-phenylpyrazoles, such as:

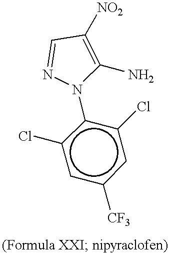

(see page 621 of “The Pesticide Manual”, 9th ed., ed. by C. R. Worthing, British Crop Protection Council, Surrey (1991));

and 3-substituted-2-aryl-4,5,6,7-tetrahydroindazoles (Lyga et al. Pesticide Sci. 42.29-36 (1994)).

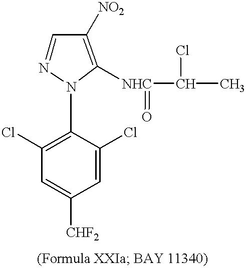

Also of significance are phenylpyrazoles of the type described in WO 96/01254 and WO 97/00246, both of which are hereby incorporated by reference. (Formula XXII).

Levels of herbicide that normally are inhibitory to the activity of protox include application rates known in the art, and that depend partly on external factors such as environment, time and method of application. For example, in the case of the imide herbicides represented by Formulae V through IX, and more particularly those represented by Formulae X through XVII, the application rates range from 0.0001 to 10 kg/ha, preferably from 0.005 to 2 kg/ha. This dosage rate or concentration of herbicide may be different, depending on the desired action and particular compound used, and can be determined by methods known in the art.

A further object of the invention is a method for controlling the growth of undesired vegetation that comprises applying to a population of the plant selected from a group consisting of Arabidopsis, sugar cane, soybean, barley, cotton, tobacco, sugar beet, oilseed rape, maize, wheat, sorghum, rye, oats,-turf and forage grasses, millet, forage and rice and the like an effective amount of a protox-inhibiting herbicide. Preferred is a method for controlling the growth of undesired vegetation, which comprises applying to a population of the selected from the group consisting of selected from the group consisting of soybean, cotton, tobacco, sugar beet, oilseed rape, maize, wheat, sorghum, rye, oats, turf grasses and rice an effective amount of a protox-inhibiting herbicide. Particularly preferred is a method for controlling the growth of undesired vegetation, which comprises applying to a population of the selected from the group consisting of Arabidopsis, soybean, cotton, sugar beet, oilseed rape, maize, wheat, sorghum, and rice.

III. Plastid Transformation and Expression

The present invention further encompasses a chimeric gene comprising a promoter capable of expression in a plant plastid operatively linked to a DNA molecule of the present invention. A preferred promoter capable of expression in a plant plastid is a promoter isolated from the 5′ flanking region upstream of the coding region of a plastid gene, which may come from the same or a different species, and the native product of which is typically found in a majority of plastid types including those present in non-green tissues. Examples of such promoters are promoters of clpP genes, such as the tobacco clpP gene promoter (WO 97/06250, incorporated herein by reference) and the Arabidopsis clpP gene promoter (U.S. application Ser. No. 09/038,878, incorporated herein by reference). Other promoters that are capable of expressing a DNA molecule in plant plastids are promoters recognized by viral RNA polymerases. Preferred promoters of this type are promoters recognized by a single sub-unit RNA polymerase, such as the T7 gene 10 promoter, which is recognized by the bacteriophage T7 DNA-dependent RNA polymerase. Yet another promoter that is capable of expressing a DNA molecule in plant plastids comes from the regulatory region of the plastid 16S ribosomal RNA operon (Harris et al., Microbiol. Rev. 58:700-754 (1994), Shinozaki et al., EMBO J. 5:2043-2049 (1986), both of which are incorporated herein by reference). The gene encoding the T7 polymerase is preferably transformed into the nuclear genome and the T7 polymerase is targeted to the plastids using a plastid transit peptide. Expression of the DNA molecules in the plastids can be constitutive or can be inducible. These different embodiment are extensively described in WO 98/11235, incorporated herein by reference. The chimeric gene preferably further comprises a 5′ untranslated sequence (5′ UTR) functional in plant plastids and a plastid gene 3′ untranslated sequence (3′ UTR) operatively linked to a DNA molecule of the present invention. Preferably, the 3′ UTR is a plastid rps16 gene 3′ untranslated sequence. In a further embodiment, the-chimeric gene comprises a poly-G tract instead of a 3′ untranslated sequence.

The present invention also encompasses a plastid transformation vector comprising the chimeric gene described above and flanking regions for integration into the plastid genome by homologous recombination. The plastid transformation vector may optionally comprise at least one chloroplast origin of replication. The present invention also encompasses a plant plastid transformed with such a plastid transformation vector, wherein the DNA molecule is expressible in the plant plastid. The invention also encompasses a plant or plant cell, including the progeny thereof, comprising this plant plastid. In a preferred embodiment, the plant is homoplasmic for transgenic plastids. The plants transformed in the present invention may be monocots or dicots. A preferred monocot is maize and a preferred dicot is tobacco. Other preferred dicots are tomato and potato.

In a preferred embodiment, the present invention encompasses a chimeric gene comprising a promoter capable of expression in a plant plastid operatively linked to a DNA molecule isolated from a prokaryote or a eukaryote that encodes a native or modified protox enzyme, such as a DNA molecule that encodes a native or modified wheat, soybean, cotton, sugar beet, rape, rice, sorghum, or sugar cane protox enzyme. Such a DNA molecule is comprised in a plastid transformation vector as described above and plants homoplasmic for transgenic plastid genomes are produced. Expression in plant plastids of a DNA molecule that encodes a modified protox enzyme preferably confers upon the plant tolerance to a herbicide in amounts that inhibit naturally occurring protox activity.

In a further preferred embodiment, the present invention encompasses a chimeric gene comprising (a) a DNA molecule isolated from a plant, which in its native state encodes a polypeptide that comprises a plastid transit peptide, and a mature enzyme that is natively targeted to a plastid of the plant by the plastid transit peptide, wherein the DNA molecule is modified such that it does not encode a functional plastid transit peptide; and (b) a promoter capable of expressing the DNA molecule in a plastid, wherein the promoter is operatively linked to the DNA molecule. In one preferred embodiment, the transit peptide is mutated and thus does not allow the proper transport of the enzyme encoded by the DNA molecule to the desired cell compartment, such as the plastid. In another preferred embodiment, a portion of the transit peptide coding sequence or the entire transit peptide coding sequence is removed from the DNA molecule, preventing the enzyme from being properly targeted to the desired cell compartment.

The chimeric genes described above are inserted in plastid transformation vectors, and the present invention is therefore also directed to plants having their plastid genome transformed with such vectors, whereby the DNA molecule is expressible in plant plastids. Such plants are preferably homoplasmic for transgenic plastids.

In a preferred embodiment, a DNA molecule described immediately above encodes an enzyme that in its wild-type form is inhibited by a herbicide. In a further preferred embodiment, the DNA molecule encodes an enzyme that in its wild-type form is inhibited by a herbicide, but that comprises at least one amino acid change compared to the wild-type enzyme. Such an amino acid change makes the enzyme resistant to compounds that naturally inhibit the wild-type enzyme. In a further preferred embodiment, the DNA molecule encodes an enzyme having protoporphyrinogen oxidase (protox) activity. In a further preferred embodiment, the transit peptide is removed from the DNA molecule as further illustrated in Examples 37-42. Plants homoplasmic for transgenic plastids of the invention are resistant to high amounts of herbicides such as Formula XVII that inhibit the naturally occurring protox activity (as further illustrated in Example 44).

In another preferred embodiment, the transit peptide of a DNA molecule encoding a 5-enolpyruvyl-3-phosphoshikimate synthase (EPSP synthase) is mutated or removed. The resulting DNA molecule is fused to a promoter capable of expression in plant plastids and homoplasmic plants harboring such constructs in their plastid genomes are obtained. These plants are resistant to herbicidal compounds that naturally inhibit EPSP synthase, in particular glyphosate. In another preferred embodiment, the transit peptide of a DNA molecule encoding a acetolactate synthase (ALS) is mutated or removed. The resulting DNA molecule is fused to a promoter capable of expression in plant plastids and homoplasmic plants harboring such constructs in their plastid genome are obtained. These plants are resistant to herbicidal compounds that naturally inhibit ALS, in particular sulfonylureas. In another preferred embodiment, the transit peptide of a DNA molecule encoding a acetoxyhydroxyacid synthase (AHAS) is mutated or removed. The resulting DNA molecule is fused to a promoter capable of expression in plant plastids and homoplasmic plants harboring such constructs in their plastid genome are obtained. These plants are resistant to herbicidal compounds that naturally inhibit AHAS, in particular, imidazolinone and sulfonamide herbicides. in another preferred embodiment, the transit peptide of a DNA molecule encoding an acetylcoenzyme A carboxylase (ACCase) is mutated or removed. The resulting DNA molecule is fused to a promoter capable of expression in plant plastids and homoplasmic plants harboring such constructs in their plastid genome are obtained. These plants are resistant to herbicidal compounds that naturally inhibit ACCase, in particular cyclohexanedione and arylphenoxypropanoic acid herbicides. In another preferred embodiment, the transit peptide of a DNA molecule encoding a glutamine synthase (GS) is mutated or removed. The resulting DNA molecule is fused to a promoter capable of expression in plant plastids and homoplasmic plants harboring such constructs in their plastid genome are obtained. These plants are resistant to herbicidal compounds that naturally inhibit GS, in particular phosphinothricin and methionine sulfoximine.

The present invention is also further directed to a method of obtaining herbicide-resistant plants by transforming their plastid genome with a chimeric gene comprising (a) a DNA molecule isolated from a plant, which in its native state encodes a polypeptide that comprises a plastid transit peptide, and a mature enzyme that is natively targeted to a plastid of the plant by the plastid transit peptide, wherein the DNA molecule is modified such that it does not encode a functional plastid transit peptide; and (b) a promoter capable of expressing the DNA molecule in a plastid, wherein the promoter is operatively linked to the DNA molecule. Examples of enzymes that are used in the present invention are cited immediately above, but the applicability of such a method is not limited to the cited examples.

The present invention is still further directed to a novel method for selecting a transplastomic plant cell, comprising the steps of: introducing the above-described chimeric gene into the plastome of a plant cell; expressing the encoded enzyme in the plastids of said plant cell; and selecting a cell that is resistant to a herbicidal compound that naturally inhibits the activity of the enzyme, whereby the resistant cell comprises transformed plastids. In a preferred embodiment, the enzyme is naturally inhibited by a herbicidal compound and the transgenic plant is able to grow on an amount of the herbicidal compound that naturally inhibits the activity of the enzyme. In a further preferred embodiment, the enzyme has protoporphyrinogen oxidase (protox) activity and is modified so that it that confers resistance to protox inhibitors.

A further aspect of the present invention is a novel method for plastid transformation of recalcitrant plants. The methods pioneered for plastid transformation of tobacco and lower plant species rely on non-lethal selection for resistance to antibiotics that preferentially affect the plastid translational apparatus and hence allow photo-heterotrophic transformants to outgrow heterotrophic, non-transformed tissue.

Several factors have likely contributed to the difficulties encountered with plastid transformation of monocots and other dicots. For example, the maize chloroplast 16S ribosomal RNA (rRNA) is naturally resistant to spectinomycin because of the presence of a G at position 1138 in the Zea mays 16S rDNA gene (Harris et al., 1994). Thus, utilization of 16s rRNA point mutations that confer spectinomycin and/or streptomycin resistance which have been used successfully as selectable chloroplast markers in Chiamydomonas and tobacco (Boynton and Gillham (1993) In Wu, R. [Ed.] Methods in Enzymology Vol 217. Academic Press, San Diego, pp. 510-536; Svab et al. (1990) Proc. Natl. Acad. Sci. U.S.A. 87: 8526-8530) is not feasible for maize. Natural spectinomycin and streptomycin resistance in maize also obviates the use of the bacterial aadA gene encoding aminoglycoside 3″ -adenyltransferase, which results in dominant spectinomycin and streptomycin resistance and allows a 100-fold increase in tobacco chloroplast transformation efficiency (Svab and Maliga (1993) Proc. Natl. Acad. Sci. U.S.A. 90: 913-917). Use of kanamycin (the only other antibiotic proven to be useful for chloroplast transformation) is also problematic due to a large excess (ca. 50:1) of nuclear vs. chloroplast-encoded resistance in tobacco following bombardment of the bacterial nptII gene encoding neomycin phosphotransferase (Carrer et al. (1993) Mol. Gen. Genet. 241: 49-56). This has been shown to result from both a high frequency of spontaneous nuclear resistance mutants as well as integration of nptII into the nuclear genome. Since nptII is also a highly effective selectable marker for maize nuclear transformation it is reasonable to expect similar background levels to that observed in tobacco. Spontaneous resistance and a significant excess of selectable marker integration by random, illegitimate recombination into the nuclear genome, rather than homologous integration into the chloroplast genome, would make recovery of bona fide chloroplast transformants difficult if not impossible.

A more fundamental reason for the difficulties encountered with plastid transformation in plant species other than tobacco may have to do with the non-photosynthetic nature of many regenerable cultured plant tissues, especially in maize and Arabidopsis. Tobacco is an exception in that cultured vegetative tissues are regenerable and contain mature differentiated chloroplasts that are photosynthetically competent in the presence of sucrose. Consequently, the current system for selecting tobacco plastid transformants relies on the faster growth rate of transformed cells that can use both reduced and inorganic carbon sources. Moreover, transformed cells do not suffer the chloroplast membrane damage that results from inhibition of plastid protein synthesis in the light. This expression of selectable markers that act preferentially on photosynthetic cells, driven by promoters that have high activity in differentiated chloroplasts, is unlikely to work in non-green tissues containing proplastids (e.g. dark-grown maize Type I callus, somatic embryos) or amyloplasts/leucoplasts (e.g. Arabidopsis root cultures). Plastid transformation in these plants requires a selectable marker that gives strong selection in all plastid types.

A preferred selectable marker for generalized plastid transformation: (1) is active only in the plastid to eliminate nuclear-transformed “escapes”; (2) has a mode of action that does not depend on photosynthetic competence or the presence of fully differentiated chloroplasts; and (3) has a level of resistance that is co-dependent on an adjustable external parameter (e.g. light), rather than being determined solely by the bulk concentration of a selective agent, so that selection pressure can vary during selection to facilitate segregation of the many-thousand plastid genome copies.

In a preferred embodiment, such a selectable marker gene involves the use of a chimeric gene comprising an isolated DNA molecule encoding a plastid-targeted enzyme having in its natural state a plastid transit peptide, wherein the DNA molecule is modified such that the transit peptide either is absent or does not function to target the enzyme to the plastid, wherein the DNA molecule is operatively linked to a promoter capable of expression in plant plastids. In a preferred embodiment, a DNA molecule of the present invention encodes an enzyme that is naturally inhibited by a herbicide. In another preferred embodiment, the DNA molecule encodes a protoporphyrinogen IX oxidase (“Protox”). In a preferred embodiment, the protoporphyrinogen IX oxidase gene is from Arabidopsis thaliana and in a more preferred embodiment, the protoporphyrinogen IX oxidase gene is from Arabidopsis thaliana and comprises at least one amino acid substitution. Preferably, an amino acid substitution results in tolerance of the enzyme against inhibition by an herbicide which naturally inhibits the activity of the enzyme. Low concentrations of herbicide are thought to kill wildtype plants due to light-sensitive intermediates which build up when the plastid-localized Protox enzyme is inhibited. Production of these photosensitizing compounds does not require differentiated chloroplasts or active photosynthesis, which is a key factor for successful plastid transformation of plants whose regenerable cultured tissues are of non-photosynthetic nature.

Another key feature is to have expression of the selectable marker gene in non-green plastids. In a preferred embodiment, the invention encompasses the use of promoters that Hare capable of expression of operatively linked DNA molecules in plastids of both green and non-green tissue. In particular, one such promoter comes from the regulatory region of the plastid 16S ribosomal RNA operon. Another candidate is the promoter and 5′ UTR from the plastid clpP gene. The clpP gene product is expressed constitutively in plastids from all plant tissues, including those that do not contain chloroplasts (Shanklin (1995) Plant Cell 7: 1713-22).

Other DNA molecules may be co-introduced in plant plastids using the method described above. In a preferred embodiment, a plastid transformation vector of the present invention contains a chimeric gene allowing for selection of transformants as described above and at least one other gene fused to a promoter capable of expression in plant plastids. The other such gene may, for example, confer resistance to insect pests, or to fungal or bacterial pathogens, or may encode one or more value-added traits.

### EXAMPLES

The invention will be further described by reference to the following detailed examples. These examples are provided for purposes of illustration only, and are not intended to be limiting unless otherwise specified. Standard recombinant DNA and molecular cloning techniques used here are well known in the art and are described by Ausubel (ed.), Current Protocols in Molecular Biology, John Wiley and Sons, Inc. (1994); T. Maniatis, E. F. Fritsch and J. Sambrook, Molecular Cloning: A Laboratory Manual, Cold Spring Harbor laboratory, Cold Spring Harbor, N.Y. (1989); and by T. J. Silhavy, M. L. Berman, and L. W. Enquist, Experiments with Gene Fusions, Cold Spring Harbor Laboratory, Cold Spring Harbor, N.Y. (1984).

### Section A. Isolation and Characterization of Plant Protoporphyrinogen Oxidase (Protox) Genes

### Example 1

Isolation of a Wheat Protox-1 cDNA Based on Sequence Homology to a Maize Protox-1 Coding Sequence

Total RNA prepared from Triticum aestivum (cv Kanzler) was submitted to Clontech for custom cDNA library construction in the Lambda Uni-Zap vector. Approximately 50,000 pfu of the cDNA library were plated at a density of approximately 5,000 pfu per 10 cm Petri dish and duplicate filter lifts were made onto nitrocellulose membranes (Schleicher and Schuell). The plaque lifts were probed with the maize Protox-1 cDNA (SEQ ID NO:5; see Example 2 of International application no. PCT/IB95/00452, filed Jun. 8, 1995, published Dec. 21, 1995 as WO 95/34659) labeled with 32P-dCTP by the random priming method (Life Technologies). Hybridization conditions were 7% sodium dodecyl sulfate (SDS), 0.5 M NaPO4 pH 7.0, 1 mM EDTA at 50° C. Wash conditions were 2×SSC, 1% SDS at 50° C. (Church and Gilbert, Proc. Natl. Acad. Sci. USA 81:1991-1995 (1984), hereby incorporated by reference in its entirety.) Positively hybridizing plaques were purified and in vivo excised into pBluescript plasmids. The sequences of the cDNA inserts were determined by the chain termination method using dideoxy terminators labeled with fluorescent dyes (Applied Biosystems, Inc.). The longest wheat Protox-1 cDNA obtained from initial screening efforts, designated “wheat Protox-1”, was 1489 bp in length. Wheat Protox-1 lacks coding sequence for the transit peptide plus approximately 126 amino acids of the mature coding sequence based on comparison with the other known plant protox peptide sequences.

A second screen was performed to obtain a longer wheat protox cDNA. For this screen, a Trifticum aestivum (cv Kanzler) cDNA library was prepared internally using the lambda Uni-Zap vector. Approximately 200,000 pfu of the cDNA library was screened as indicated above, except that the wheat Protox-1 cDNA was used as a probe and hybridization and wash conditions were at 65° C. instead of 50° C. The longest wheat cDNA obtained from this screening effort, designated “wheat Protox-1a”, was 1811 bp in length. The nucleotide sequence of this cDNA and the amino acid sequence it encodes are set forth in SEQ ID NOs:9 and 10, respectively. Based on comparison with the other known plant protox peptide sequences and with corresponding genomic sequence, this cDNA is either full-length or missing only a few transit peptide codons (Table 1). This wheat protein sequence is 91% identical (95% similar) to the maize Protox-1 protein sequence set forth in SEQ ID NO:6.

Wheat Protox-1a, in the pBluescript SK vector, was deposited Mar. 19, 1996, as pWDC-13 (NRRL #B21545).

### Example 2

Isolation of a Soybean Protox-1 cDNA Based on Sequence Homology to an Arabidopsis Protox-1 Coding Sequence

A Lambda Uni-Zap cDNA library prepared from soybean (v Williams 82, epicotyls) was purchased from Stratagene. Approximately 50,000 pfu of the library was plated at a density of approximately 5,000 pfu per 10 cm Petri dish and duplicate filter lifts were made onto Colony/Plaque Screen membranes (NEN Dupont). The plaque lifts were probed with the Arabidopsis Protox-1 cDNA (SEQ ID NO:1; see Example 1 of International application no. PCT/IB95/00452, filed Jun. 8, 1995, published Dec. 21, 1995 as WO 95/34659)) labeled with 32P-dCTP by the random priming method (Life Technologies). Hybridization conditions were 7% sodium dodecyl sulfate (SDS), 0.5 M NaPO4 pH 7.0, 1 mM EDTA at 50° C. Wash conditions were 2×SSC, 1% SDS at 50° C. (Church and Gilbert (1984)). Positively hybridizing plaques were purified and in vivo excised into pBluescript plasmids. The sequence of the cDNA inserts was determined by the chain termination method using dideoxy terminators labeled with fluorescent dyes (Applied Biosystems, Inc.). The longest soybean cDNA obtained, designated “soybean Protox-1”, is full-length based on comparison with the other known plant protox peptide sequences (Table 1). Soybean Protox-1 is 1847 bp in length and encodes a protein of 58.8 kDa. The nucleotide sequence of this cDNA and the amino acid sequence it encodes are set forth in SEQ ID NOs:11 and 12, respectively. The soybean protein is 78% identical (87% similar) to the Arabidopsis Protox-1 protein.

Soybean Protox-1, in the pBluescript SK vector, was deposited Dec. 15, 1995 as pWDC-12 (NRRL #B-21516).

An alignment of the predicted amino acid sequences of the respective proteins encoded by the coding sequences shown in SEQ ID NOs: 1, 5, 9, 11, 15, 17, 19, 21, 23 and 36 are set forth in Table 1. An alignment of the predicted amino acid sequences of the respective proteins encoded by the sequences shown in SEQ ID NOS: 3 and 7 are set forth in Table 2.

TABLE 1

Comparison of full-length and partial Protox-1 Amino Acid
Sequences from Arabidopsis (“Arabpt-1”; SEQ ID NO:2),
Maize (“Mzpt-1”; SEQ ID NO:6), Wheat (“Wtpt-1”;
SEQ ID NO:10), Soybean (“Soybeanpt-1”; SEQ ID NO:12),
Cotton (“Cottonpt-1”; SEQ ID NO:16), Sugar beet (“Sugpt-1”;
SEQ ID NO:18), Rape (“Rapept-1”; SEQ ID NO:20), Rice
(“Ricept-1”; SEQ ID NO:22), Sorghum (“Sorghumpt-1”;
SEQ ID NO:24), and Sugar cane (“Scpt-1”; SEQ ID NO:37).
Alignment was performed using the PileUp program (GCG package,
University of Wisconsin, Madison, WI). Positions that may be
modified according to the teachings herein to confer or enhance
inhibitor resistance are shown in bold type.

| | |
|---|---|
| | 1 50 |
| Rapept-1 | .......... .......... MDLSLLRP.. QPFLSPFSNP FPRSRPYKPL |
| Arabpt-1 | .......... .......... MELSLLRPTT QSLLPSFSKF NLRLNVYKPL |
| Sorghumpt-1 | .......... .......... .......... .......... .......... |
| Mzpt-1 | .......... .......... .......... .......... .......... |
| Wtpt-1 | .......... .......... .........M ATATVAAASP LRGRVTGRPH |
| Ricept-1 | .......... .......... .......... .......... .......... |
| Cottonpt-1 | .......... ......MTAL IDLSLLRSSP SVSPFSIPHH QHPPRFRKPF |
| Soybeanpt1 | ........MV SVFNEILFPP NQTLLRPSLH SPTSFFTSPT RKFPRSRPNP |
| Sugpt-1 | MKSMALSNCI PQTQCMPLRS SGHYRGNCIM LSIPCSLIGR RGYYSHKKPR |
| Scpt-1 | .......... .......... .......... .......... .......... |
| | 51 100 |
| Rapept-1 | NLRCSVSGGS VVGSSTIEGG GGGKTVTADC VIVGGGISGL CIAQALVTKH |
| Arabpt-1 | RLRCSVAGGP TVGSSKIEGG GGT.TITTDC VIVGGGISGL CIAQALATKH |
| Sorghumpt-1 | .......... .......... .......... .......... .......... |
| Mzpt-1 | .......... .......... .......ADC VVVGGGISGL CTAQALATRH |
| Wtpt-1 | RVRPRCATAS SATETPAAPG VRL...SAEC VIVGAGISGL CTAQALATRY |
| Ricept-1 | .......... .......... .......... .......... .......... |
| Cottonpt-1 | KLRCSLAEGP TISSSKIDGG ESS...IADC VIVGGGISGL CIAQALATKH |
| Soybeanpt1 | ILRCSIAEES TASPPKTR.. DSA...PVDC VVVGGGVSGL CIAQALATKH |
| Sugpt-1 | MSMSCSTSSG SKSAVKEAGS GSGAGGLLDC VIVGGGISGL CIAQALCTKH |
| Scpt-1 | .......... .......... .......... .......... .......... |
| | 101................................................150 |
| Rapept-1 | PDA..AKNVM VTEAKDRVGG NIIT..REEQ GFLWEEGPNS FQPSDPMLTM |
| Arabpt-1 | PDA..APNLI VTEAKDRVGG NIIT..REEN GFLWEEGPNS FQPSDPMLTM |
| Sorghumpt-1 | .......... .......... ..STVERPEE GYLWEEGPNS FQPSDPVLSM |
| Mzpt-1 | ..G..VGDVL VTEARARPGG NITTVERPEE GYLWEEGPNS FQPSDPVLTM |
| Wtpt-1 | ..G..VSDLL VTEARDRPGG NITTVERPDE GYLWEEGPNS FQPSDPVLTM |
| Ricept-1 | .......... .......... .......... .......... .......... |
| Cottonpt-1 | RDV..ASNVI VTEARDRVGG NITTVER..D GYLWEEGPNS FQPSDPILTM |
| Soybeanpt1 | ..A..NANVV VTEARDRVGG NITTMER..D GYLWEEGPNS FQPSDPMLTM |
| Sugpt-1 | SSSSLSPNFI VTEAKDRVGG NIVTVE..AD GYIWEESPNS FQPSDAVLTM |
| Scpt-1 | .......... .......... .......... .......... .......... |
| | 151 200 |
| Rapept-1 | VVDSGLKDDL VLGDPTAPRF VLWNGKLRPV PSKLTDLPFF DLMSIGGKIR |
| Arabpt-1 | VVDSGLKDDL VLGDPTAPRF VLWNGKLPRV PSKLTDLPFF DLMSIGGKIR |
| Sorghumpt-1 | AVDSGLKDDL VFGDPNAPRF VLWEGKLRPV PSKPADLPFF DLMSIPGKLR |
| Mzpt-1 | AVDSGLKDDL VFGDPNAPRF VLWEGKLRPV PSKPADLPFF DLMSIPGKLR |
| Wtpt-1 | AVDSGLKDDL VFGDPNAPRF VLWEGKLRPV PSKPGDLPFF SLMSIPGKLR |
| Ricept-1 | .......... .......... .......... .......... .......... |
| Cottonpt-1 | AVDSGLKDDL VLGDPNAPRF VLWEGKLRPV PSKPTDLPFF DLMSIAGKLR |
| Soybeanpt1 | VVDSGLKDEL VLGDPDAPRF VLWNRKLRPV PGKLTDLPFF DLMSIGGKIR |
| Sugpt-1 | AVDSGLKDEL VLGDPNAPRF VLWNDKLRPV PSSLTDLPFF DLMTIPGKIR |
| Scpt-1 | .......... .......... .......... .......... .......... |
| | 201 250 |
| Rapept-1 | AGFGAIGIRP SPPGREESVE EFVRRNLGDE VEERLIEPFC SGVYAGDPAK |
| Arabpt-1 | AGFGALGIRP SPPGREESVE EFVRRNLGDE VFERLIEPFC SGVYAGDPSK |
| Sorghumpt-1 | AGLGALGIRP PAPGREESVE EFVRRNLGAE VFERLIEPFC SGVYAGDPSK |
| Mzpt-1 | AGLGALGIRP PPPGREESVE EFVRRNLGAE VFERLIEPFC SGVYAGDPSK |
| Wtpt-1 | AGLGALGIRP PPFGREESVE EFVRRNLGAE VFERLIEPFC SGVYAGDPSK |
| Ricept-1 | .......... .......... .......... .......... .......... |
| Cottonpt-1 | AGFGAIGIRP PPPGYEESVE EFVRRNLGAE VFERFIEPFC SGVYAGDPSK |
| Soybeanpt1 | AGFGALGIRP PPPGHEESVE EFVRPNLGDE VFERLIEPFC SGVYAGDPSK |
| Sugpt-1 | AALGALGFRP SPPPHEESVE HFVRRNLGDE VFERLIEPFC SGVYAGDPAK |
| Scpt-1 | .......... .......... .......... .......... .......... |
| | 251 300 |
| Rapept-1 | LSMKAAFGKV WKLEENGGSI IGGAFKAIQA KNKAPKTTRD PRLPKPKGQT |
| Arabpt-1 | LSMKAAFGKV WKLEQNGGSI IGGTFKAIQE RKNAPKAERD PRLPKPQGQT |
| Sorghumpt-1 | LSMKAAFGKV WPLEEAGGSI IGGTIKTIQE RGKNPKPPRD PRLPKPKGQT |
| Mzpt-1 | LSMKAAFGKV WPLEETGGSI IGGTIKTIQE RSKNPKPPRD ARLPKPKGQT |
| Wtpt-1 | LSMKAAFGKV WRLEEIGGSI IGGTIKAIQD KGKNPKPFRD PPLPAPKGQT |
| Ricept-1 | RALKAAFGKV WRLEDTGGSI IGGTIKTIQE RGKNPKPPRD PRLPTPKGQT |
| Cottonpt-1 | LSMKAAFGRV WKLEEIGGSI IGGTFKTIQE RNKTPKPPRD PRLPKPKGQT |
| Soybeanpt1 | LSMKAAFGKV WKLEKNGGSI IGGTFKTIQE RNGASKPPRD PRLPKPKGQT |
| Sugpt-1 | LSMKAAFGKV WKLEQKGGSI IGGTLKAIQE RGSNPKPPRD QRLPKPKGQT |
| Scpt-1 | .......... .......... .......... .......... .......... |
| | 301 350 |
| Rapept-1 | VGSFRKGLTM LPEAISARLG DKVKVSWKLS SITKLASGEY SLTYETPEGI |
| Arabpt-1 | VGSFRKGLRM LPEAISARLG SKVKLSWKLS GITKLESGGY NLTYETPDGL |
| Sorghumpt-1 | VASFRKGLAM LPNAITSSLG SKVKLSWKLT SMTKSDGKGY VLEYETPEGV |
| Mzpt-1 | VASFPKGLAM LPNAITSSLG SKVKLSWKLT SITKSDDKGY VLEYETPEGV |
| Wtpt-1 | VASFRKGLAM LPNAIASRLG SKVKLSWKLT SITKADNQGY VLGYETPEGL |
| Ricept-1 | VASFRKGLTM LPDAITSRLG SKVKLSWKLT SITKSDNKGY ALVYETPEGV |
| Cottonpt-1 | VGSFRKGLTM LPEAIANSLG SNVKLSWKLS SITKLGNGGY NLTFETPEGM |
| Soybeanpt1 | VGSFRKGLTM LPDAISARLG NKVKLSWKLS SISKLDSGEY SLTYETPEGV |
| Sugpt-1 | VGSFRKGLVM LPTAISARLG SRVKLSWTLS SIVKSLNGEY SLTYDTPDGL |
| Scpt-1 | .......... .......... .......... .......... .......... |
| | 351 400 |
| Rapept-1 | VTVQSKSVVM TVPSHVASSL LRPLSDSAAE ALSKLYYPPV AAVSISYAKE |
| Arabpt-1 | VSVQSKSVVM TVPSHVASGL LRPLSESAAN ALSKLYYPPV AAVSISYPKE |
| Sorghumpt-1 | VLVQAKSVIM TIPSYVASDI LRPLSGDAAD VLSRFYYPPV AAVTVSYPKE |
| Mzpt-1 | VSVQAKSVIM TIPSYVASNI LRPLSSDAAD ALSRFYYPPV AAVTVSYPKE |
| Wtpt-1 | VSVQAKSVIM TIPSYVASDI LRPLSIDAAD ALSKFYYPPV AAVTVSYPKE |
| Ricept-1 | VSVQAKTVVM TIPSYVASDI LRPLSSDAAD ALSIFYYPPV AAVTVSYPKE |
| Cottonpt-1 | VSLQSRSVVM TIPSHVASNL LHPLSAAAAD ALSQFYYPPV ASVTVSYPKE |
| Soybeanpt1 | VSLQCKTVVL TIPSYVASTL LRPLSAAAAD ALSKFYYPPV AAVSISYPKE |
| Sugpt-1 | VSVRTKSVVM TVPSYVASPL LRPLSDSAAD SLSKFYYPPV AAVSLSYPKE |
| Scpt-1 | .......... .......... .......... .......... .......... |
| | 401 450 |
| Rapept-1 | AIRSECLIDG ELKGFGQLHP RTQKVETLGT IYSSSLFPNR APPGRVLLLN |
| Arabpt-1 | AIRTECLIDG ELKGFGQLHP RTQGVETLGT IYSSSLFPNR APPGRILLLN |
| Sorghumpt-1 | AIRKECLIDG ELQGFGQLHP RSQGVETLGT IYSSSLFPNR APAGRVLLLN |
| Mzpt-1 | AIRKECLIDG ELQGFGQLHP RSQGVETLGT IYSSSLFPNR APDGRVLLLN |
| Wtpt-1 | AIRKECLIDG ELQGFGQLHP RSQGVETLGT IYSSSLFPNR APAGRVLLLN |
| Ricept-1 | AIRKECLIDG ELQGFGQLHP RSQGVETLGT IYSSSLFPNR APAGRVLLLN |
| Cottonpt-1 | AIRKECLIDG ELKGFGQLHP RSQGIETLGT IYSSSLFPNR APSGRVLLLN |
| Soybeanpt1 | AIRSECLIDG ELKGFGQLHP RSQGVETLGT IYSSSLFPNR APPGRVLLLN |
| Sugpt-1 | AIRSECLING ELQGFGQLHP RSQGVETLGT IYSSSLFPNR APPGRILILS |
| Scpt-1 | .......... .......... .......... .......... .......... |
| | 451 500 |
| Rapept-1 | YIGGATNTGI LSKSEGELVE AVDRDLRKML IKPSSTDPLV LGVKLWPQAI |
| Arabpt-1 | YIGGSTNTGI LSKSEGELVE AVDRDLRKML IKPNSTDPLK LGVRVWPQAI |
| Sorghumpt-1 | YIGGATNTGI VSKTESELVE AVDRDLRKML INPTAVDPLV LGVRVWPQAI |
| Mzpt-1 | YIGGATNTGI VSKTESELVE AVDRDLRKML INSTAVDPLV LGRVRWPQAI |
| Wtpt-1 | YIGGSTNTGI VSKTESDLVG AVDRDLRKML INPRAADPLA LGVRVWPQAI |
| Ricept-1 | YIGGSTNTGI VSKTESELVE AVDRDLRKML INPRAVDPLV LGVRVWPQAI |
| Cottonpt-1 | YIGGATNTGI LSKTEGELVE AVDRDLRKML INPNAKDPLV LGVRVWPKAI |
| Soybeanpt1 | YIGGATNTGI LSKDESELVE TVDRDLRKIL INPNAQDPFV VGVRLWPQAI |
| Sugpt-1 | YIGGAKNPGI LNKSKDELAK TVDKDLRRML INPDAKLPRV LGVRVWPQAI |
| Scpt-1 | .......... .SKTESELVE AVDRDLRKML INPTAVDPLV LGVRVWPQAI |
| | 501 550 |
| Rapept-1 | PQFLIGHIDL VDAAKASLSS SGHEGLFLGG NYVAGVALGR CVEGAYETAT |
| Arabpt-1 | PQFLVGHFDI LDTAKSSLTS SGYEGLFLGG NYVAGVALGR CVEGAYETAI |
| Sorghumpt-1 | PQFLVGHLDL LEAAKSALDQ GGYNGLFLGG NYVAGVALGR CIEGAYESAA |
| Mzpt-1 | PQFLVGHLDL LEAAKAALDR GGYFGLFLGG NYVAGVALGR CVEGAYESAS |
| Wtpt-1 | PQFLIGHLDR LAAAKSALGQ GGYDGLFLGG KYVAGVALGR CIEGAYESAS |
| Ricept-1 | PQFLIGHLDH LEAAKSALGK GGYDGLFLGG NYVAGVALGR CVEGAYESAS |
| Cottonpt-1 | PQFLVGHLDL LDSAKMALRD SGFHGLFLGG NYVSGVALGR CVEGAYEVAA |
| Soybeanpt1 | PQFLVGHLDL LDVAKASIRN TGFEGLFLGG NYVSGVALGR CVEGAYEVAA |
| Sugpt-1 | PQFSIGHFDL LDAAKAALTD TGVKGLFLGG NYVSGVALGR CIEGAYESAA |
| Scpt-1 | PQFLVGHLDL LEAAKSALDR GGYDGLFLGG NYVAGVALGR CVEGAYESAS |
| | 551 563 |
| Rapept-1 | QVNDFMSRYA YK* |
| Arabpt-1 | EVNNFMSRYA YK* |
| Sorghumpt-1 | QIYDFLTKYA YK* |
| Mzpt-1 | QISDFLTKYA YK* |
| Wtpt-1 | QVSDFLTKYA YK* |
| Ricept-1 | QISDYLTKYA YK* |
| Cottonpt-1 | EVKEFLSQYA YK* |
| Soybeanpt1 | EVNDFLTNRV YK* |
| Sugpt-1 | EVVDFLSQYS DK* |
| Scpt-1 | QIYDFLTKYA YK* |

TABLE 2

Comparison of the Arabidopsis (SEQ ID NO:4) and
Maize (SEQ ID NO:8) Protox-2 Amino Acid Sequences
Identical residues are denoted by the vertical bar between the
two sequences. Alignment is performed using the GAP program
described in Deveraux et al., Nucleic Acids Res. 12:387-395
(1984).
Percent Similarity: 75.889 Percent Identity: 57.905
Protox-2.Pep × Mzprotox-2.Pep

| | | |
|---|---|---|
| 1 | ............................MASGAVAD.HQIEAVSGKRVAV | 21 |
| | .\| \|:\|: .: \|..::.\|\|\| | |
| 1 | MLALTASASSASSHPYRHASAHTRRPRLRAVLAMAGSDDPRAAPARSVAV | 50 |
| 22 | VGAGVSGLAAAYKLKSRGLNVTVFEADGRVGGKLRSVMQNGLIWDEGANT | 71 |
| | \|\|\|\|\|\|\|\|\|\|\|\|:\|: .\|:\|\|\|\|\|\|\|.:\|.\|\|\|:\|. :.\|::\|\|\|\|\|\|\| | |
| 51 | VGAGVSGLAAAYRLRQSGVNVTVFEAADRAGGKIRTNSEGGFVWDEGANT | 100 |
| 72 | MTEAEPEVGSLLDDLGLREKQQFPISQKKRYIVRNGVPVMLPTNPIELVT | 121 |
| | \|\|\|:\| \|.:.\|:\|\|\|\|\|.:\|\|\|:\| \|\|.\|\|\|\|\|::\|.\|.::\|.:\|\|.\|:. | |
| 101 | MTEGEWEASRLIDDLGLQDKQQYPNSQHKRYIVKDGAPALIPSDPISLMK | 150 |
| 122 | SSVLSTQSKFQILLEPFLWKK....KSSKVSDASAEESVSEFFQRHFGQE | 167 |
| | \|\|\|\|\|\|.\|\|:.:::\|\|\|\|:\|\| .\|:\|\|\|:. .\|\|\|:.\| :\|\|\|\|.\| | |
| 151 | SSVLSTKSKIALFFEPFLYKKANTRNSGKVSEEHLSESVGSFCERHFGRE | 200 |
| 168 | VVDYLTDPFVGGTSAADPDSLSMKHSFPDLWNVEKSFGSIIVGAIRTKFA | 217 |
| | \|\|\|\|::\|\|\|\|:\|\|\|\|:\|\|:\|\|\|::\|.\|\|.\|\|\|:\|:.:\|\|:\|\|\|\|\| .\|:\| | |
| 201 | VVDYFVDPFVAGTSAGDPESLSIRHAFPALWNLERKYGSVIVGAILSKLA | 250 |
| 218 | AKGGKSRDTKSSPGTKKGSRGSFSFKGGMQILPDTLCKSLSHDEINLDSK | 267 |
| | \|\|\|:. :. ..\|.\|.::..\|.\|\|\|\|.\|\|\|\| \| :.\| ..::.\|::.\|:.. | |
| 251 | AKGDPVKTRHDSSGKRRNRRVSFSFHGGMQSLINALHNEVGDDNVKLGTE | 300 |
| 268 | VLSLS..YNSGSRQENWSLSCVSHNETQRQ...NPHYDAVIMTAPLCNVK | 312 |
| | \|\|\|\|. :::.. :.\|\|:\|. \|.:..::: \|. :\|\|\|\|\|\|\|\|\|:\|\|: | |
| 301 | VLSLACTFDGVPALGRWSISVDSKDSGDKDLASNQTFDAVIMTAPLSNVR | 350 |
| 313 | EMKVMKGGQPFQLNFLPEINYMPLSVLITTFTKEKVKRPLEGFGVLIPSK | 362 |
| | \|\|. \|\|\|.\|. \|:\|\|\|.::\|:\|\|\|:::\|.\|.\|:.\|\|:\|\|\|\|\|\|\|\|\|\| \| | |
| 351 | RMKFTKGGAPVVLDFLPKMDYLPLSLMVTAFKKDDVKKPLEGFGVLIPYK | 400 |
| 363 | E.QKHGFKTLGTLFSSMMFPDRSPSDVHLYTTFIGGSRNQELAKASTDEL | 411 |
| | \| \|\|\|\|:\|\|\|\|\|\|\|\|\|\|\|\|\|\|\|.\|.\| .\|\|\|\|\|:\|\|\|:\|.:\|\| \|.\|. \| | |
| 401 | EQQKHGLKTLGTLFSSMMFPDRAPDDQYLYTTFVGGSHNRDLAGAPTSIL | 450 |
| 412 | KQVVTSDLQRLLGVEGEPVSVNHYYWRKAFPLYDSSYDSVMEAIDKMEND | 461 |
| | \|\|:\|\|\|\|\|.:\|\|\|\|\|\|:\|. \|. \|\| .\|\|\|\|\|: .\|.\|\|:\|\|\|:\|\|\|.: | |
| 451 | KQLVTSDLKKLLGVEGQPTFVKHVYWGNAFPLYGHDYSSVLEAIEKMEKN | 500 |
| 462 | LPGFFYAGNHRGGLSVGKSIASGCKAADLVISYLESCSNDKKPNDSL* | 509 |
| | \|\|\|\|\|\|\|\|\| ::\|\|.\|\|. \|\|\|\|:\|\|\|\|\|.\|\|\|\|\|\| ......: | |
| 501 | LPGFFYAGNSKDGLAVGSVIASGSKAADLAISYLESHTKHNNSH*... | 545 |

### Example 3

Isolation of a Cotton Protox-1 CDNA Based on Sequence Homology to a Maize Protox-1 Coding Sequence

A Lambda Uni-Zap cDNA library prepared from Gossypium hirsutum L. (72 hr. dark grown cotyledons) was obtained from Dr. Dick Trelease, Dept. of Botany, Arizona State University (Ni W. and Trelease R. N., Arch. Biochem. Biophys. 289: 237-243 (1991)). Approximately 50,000 pfu of the library was plated at a density of approximately 5,000 pfu per 10 cm Petri dish and duplicate filter lifts were made onto Colony/Plaque Screen membranes (NEN Dupont). The plaque lifts were probed with the maize Protox-1 cDNA (SEQ ID NO:5) labeled with 32P-dCTP by the random priming method (Life Technologies). Hybridization conditions were 7% sodium dodecyl sulfate (SDS), 0.5 M NaPO4 pH 7.0, 1 mM EDTA at 50° C. Wash conditions were 2×SSC, 1% SDS at 50° C. (Church and Gilbert (1984)). Positively hybridizing plaques were purified and in vivo excised into pBluescript plasmids. The sequence of the cDNA inserts was determined by the chain termination method using dideoxy terminators labeled with fluorescent dyes (Applied Biosystems, Inc.). The longest cotton cDNA obtained, designated “cotton Protox-1”, appears to be full-length based on comparison with the other known plant protox peptide sequences (Table 1). Cotton Protox-1 is 1826 bp in length and encodes a protein of 58.2 kDa. The nucleotide sequence of this cDNA and the amino acid sequence it encodes are set forth in SEQ ID NOs:13 and 14, respectively. The cotton protein is 77% identical (86% similar) to the Maize Protox-1 protein.

Cotton Protox-1, in the pBluescript SK vector, was deposited Jul. 1, 1996 as pWDC-15 (NRRL #B-21594).

### Example 4

Isolation of a Sugar Beet Protox-1 cDNA Based on Sequence Homology to an Arabidopsis Protox-1 Coding Sequence

A Lambda-Zap cDNA library prepared from Beta vulgaris was obtained from Dr. Philip Rea, Dept. of Botany, Plant Science Institute, Philadelphia, Pa. (Yongcheol Kim, Eugene J. Kim, and Philip A. Rea, Plant Physiol. 106: 375-382 (1994)). Approximately 50,000 pfu of the cDNA library were plated at a density of approximately 5,000 pfu per 10 cm Petri dish and duplicate filter lifts were made onto nitrocellulose membranes (Schleicher and Schuell). The plaque lifts were probed with the Arabidopsis Protox-1 cDNA (SEQ ID NO:1) labeled with 32P-dCTP by the random priming method (Life Technologies). Hybridization conditions were 7% sodium dodecyl sulfate (SDS), 0.5 M NaPO4 pH 7.0, 1 mM EDTA at 50° C. Wash conditions were 2×SSC, 1% SDS at 50° C. (Church and Gilbert (1984)). Positively hybridizing plaques were purified and in vivo excised into pBluescript plasmids. The sequences of the cDNA inserts were determined by the chain termination method using dideoxy terminators labeled with fluorescent dyes (Applied Biosystems, Inc.). The longest sugar beet Protox-1 cDNA obtained, designated “sugar beet Protox-1”, is full-length based on comparison with the other known plant protox peptide sequences (Table 1). Sugar beet Protox-1 is 1910 bp in length and encodes a protein of 60 kDa. The nucleotide sequence of this cDNA and the amino acid sequence it encodes are set forth in SEQ ID NOs:15 and 16, respectively. The sugar beet protein is 73% identical (82% similar) to the Arabidopsis Protox-1 protein.

Sugar beet Protox-1, in the pBluescript SK vector, was deposited Jul. 29, 1996, as pWDC-16 (NRRL #B-21595N).

### Example 5

Isolation of a Rape Protox-1 cDNA Based on Sequence Homology to an Arabidopsis Protox-1 Coding Sequence

A Lambda Uni-Zap II cDNA library prepared from Brassica napus (3-4 wk. mature green leaves) was obtained from Dr. Guenther Ochs, Institut Fuer Allgemeine Botanik, Johannes Gutenberg-Universitaet Mainz, Germany (Günther Ochs, Gerald Schock, and Aloysius Wild, Plant Physiol. 103: 303-304 (1993)). Approximately 50,000 pfu of the cDNA library were plated at a density of approximately 5,000 pfu per 10 cm Petri dish and duplicate filter lifts were made onto nitrocellulose membranes (Schleicher and Schuell). The plaque lifts were probed with the Arabidopsis Protox-1 cDNA (SEQ ID NO:1) labeled with 32P-dCTP by the random priming method (Life Technologies). Hybridization conditions were 7% sodium dodecyl sulfate (SDS), 0.5 M NaPO4 pH 7.0, 1 mM EDTA at 50° C. Wash conditions were 2×SSC, 1% SDS at 50° C. (Church and Gilbert (1984)). Positively hybridizing plaques were purified and in vivo excised into pBluescript plasmids. The sequences of the cDNA inserts were determined by the chain termination method using dideoxy terminators labeled with fluorescent dyes (Applied Biosystems, Inc.). The longest rape Protox-1 cDNA obtained, designated “rape Protox-1”, is full-length based on comparison with the other known plant protox peptide sequences (Table 1). Rape Protox-1 is 1784 bp in length and encodes a protein of 57.3kD. The nucleotide sequence of this cDNA and the amino acid sequence it encodes are set forth in SEQ ID NOs: 17 and 18, respectively. The rape protein is 87% identical (92% similar) to the Arabidopsis Protox-1 protein.

Rape Protox-1, in the pBluescript SK vector, was deposited Aug. 23, 1996, as pWDC-17 (NRRL #B-21615).

### Example 6

Isolation of a Rice Protox-1 cDNA Based on Sequence Homology to a Maize Protox-1 Coding Sequence

A Lambda gt11 cDNA library prepared from Oryza sativa (5 day etiolated shoots) was purchased from Clontech. Approximately 50,000 pfu of the cDNA library were plated at a density of approximately 5,000 pfu per 10 cm Petri dish and duplicate filter lifts were made onto nitrocellulose membranes (Schleicher and Schuell). The plaque lifts were probed with the maize Protox-1 cDNA (SEQ ID NO:5) labeled with 32P-dCTP by the random priming method (Life Technologies). Hybridization conditions were 7% sodium dodecyl sulfate (SDS), 0.5 M NaPO4 pH 7.0, 1 mM EDTA at 50° C. Wash conditions were 2×SSC, 1% SDS at 50° C. (Church and Gilbert (1984)). Positively hybridizing plaques were purified, and lambda DNA was prepared using the Wizard Lambda-Prep kit (Promega). The cDNA inserts were subcloned as EcoRI fragments into the pBluescript SK vector using standard techniques. The sequences of the cDNA inserts were determined by the chain termination method using dideoxy terminators labeled with fluorescent dyes (Applied Biosystems, Inc.). The longest rice Protox-1 cDNA obtained, designated “rice Protox-1”, was 1224 bp in length. Rice Protox-1 lacks coding sequence for the transit peptide plus approximately 172 amino acids of the mature coding sequence based on comparison with the other known plant protox peptide sequences (Table 1). The nucleotide sequence of this partial cDNA and the amino acid sequence it encodes are set forth in SEQ ID NOs:19 and 20, respectively.

Rice Protox-1, in the pBluescript SK vector, was deposited Dec. 6, 1996, as pWDC-18 (NRRL #B-21648).

### Example 7

Isolation of a Sorghum Protox-1 cDNA Based on Sequence Homology to a Maize Protox-1 Coding Sequence

A Lambda-Zap II cDNA library prepared from Sorghum bicolor (3-6 day green seedlings) was obtained from Dr. Klaus Pfizenmaier, Institute of Cell Biology and Immunology, University of Stuttgart, Germany (Harald Wajant, Karl-Wolfgang Mundry, and Klaus Pfizenmaier, Plant Mol. Biol. 26: 735-746 (1994)). Approximately 50,000 pfu of the cDNA library were plated at a density of approximately 5,000 pfu per 10 cm Petri dish and duplicate filter lifts were made onto nitrocellulose membranes (Schleicher and Schuell). The plaque lifts were probed with the maize Protox-1 cDNA (SEQ ID NO:5) labeled with 32P-dCTP by the random priming method (Life Technologies). Hybridization conditions were 7% sodium dodecyl sulfate (SDS), 0.5 M NaPO4 pH 7.0, 1 mM EDTA at 50° C. Wash conditions were 2×SSC, 1% SDS at 50° C. (Church and Gilbert (1984)). Positively hybridizing plaques were purified and in vivo excised into pBluescript plasmids. The sequences of the cDNA inserts were determined by the chain termination method using dideoxy terminators labeled with fluorescent dyes (Applied Biosystems, Inc.). The longest sorghum Protox-1 cDNA obtained, designated “sorghum Protox-1″, was 1590 bp in length. Sorghum Protox-1 lacks coding sequence for the transit peptide plus approximately 44 amino acids of the mature coding sequence based on comparison with the other known plant protox peptide sequences (Table 1). The nucleotide sequence of this partial cDNA and the amino acid sequence it encodes are set forth in SEQ ID NOs:21 and 22, respectively.

Sorghum Protox-1, in the pBluescript SK vector, was deposited Dec. 6, 1996, as pWDC-19 (NRRL #B-21649).

### Example 8

Isolation of a Sugar cane Protox-1 cDNA Based on Sequence Homology to a Maize Protox-1 Coding Sequence

A Lambda-Zap II cDNA library prepared from sugar cane was obtained from Henrik Albert of USDA/ARS at the Hawaii Agricultural Research Center. Approximately 50,000 pfu of the cDNA library were plated at a density of approximately 5,000 pfu per 10 cm Petri dish and duplicate filter lifts were made onto nitrocellulose membranes (Schleicher and Schuell). The plaque lifts were probed with the maize Protox-1 cDNA (SEQ ID NO:5) labeled with 32P-dCTP by the random priming method (Life Technologies). Hybridization conditions were 7% sodium dodecyl sulfate (SDS), 0.5 M NaPO4 pH 7.0, 1 mM EDTA at 50° C. Wash conditions were 2×SSC, 1% SDS at 50° C. (Church and Gilbert (1984)). Positively hybridizing plaques were purified and in vivo excised into pBluescript plasmids. The sequences of the cDNA inserts were determined by the chain termination method using dideoxy terminators labeled with fluorescent dyes (Applied Biosystems, Inc.). The longest sugar cane Protox-1 cDNA obtained, designated “sugar cane Protox-1”, was 633 bp in length. Sugar cane Protox-1 lacks coding sequence for the transit peptide plus approximately 382 amino acids of the mature coding sequence based on comparison with the other known plant protox peptide sequences (Table 1). The nucleotide sequence of this partial cDNA and the amino acid sequence it encodes are set forth in SEQ ID NOs:36 and 37, respectively.

### Example 9

Demonstration of Plant Protox Clone Sensitivity to Protox Inhibitory Herbicides in a Bacterial System

Liquid cultures of Protox-1/SASX38, Protox-2/SASX38 and pBluescript/XL1-Blue were grown in L amp¹⁰⁰. One hundred microliter aliquots of each culture were plated on L amp¹⁰⁰ media containing various concentrations (1.0 nM-10 mM) of a protox inhibitory aryluracil herbicide of formula XVII. Duplicate sets of plates were incubated for 18 hours at 37° C.

The protox⁺E. coli strain XL1-Blue showed no sensitivity to the herbicide at any concentration, consistent with reported resistance of the native bacterial enzyme to similar herbicides. The Protox-1/SASX38 was clearly sensitive, with the lawn of bacteria almost entirely eliminated by inhibitor concentrations as low as 1OnM. The Protox-2/SASX38 was also sensitive, but only at a higher concentration (10 μM) of the herbicide. The herbicide was effective even on plates maintained almost entirely in the dark. The toxicity of the herbicide was entirely eliminated by the addition of 20 μg/ml hematin to the plates.

The different herbicide tolerance between the two plant Protox strains is likely the result of differential expression from these two plasmids, rather than any inherent difference in enzyme sensitivity. Protox-1/SASX38 grows much more slowly than Protox-2/SASX38 in any heme-deficient media. In addition, the MzProtox-2/SASX38 strain, with a growth rate comparable to Arab Protox-1/SASX38, is also very sensitive to herbicide at the lower (10-100 nM) concentrations.

### Section B: Identification and Characterization of Plant Protox Genes Resistant to Protox-Inhibitory Herbicides

### Example 10

Selecting for Plant Protox Genes Resistant to Protox-Inhibitory Herbicides in the E. coli Expression System

An Arabidopsis thaliana (Landsberg) cDNA library in the plasmid vector pFL61 (Minet et al., Plant J. 2:417-422 (1992) was obtained and amplified. The E. coli hemG mutant SASX38 (Sasarman et al., J. Gen. Microbiol. 113:297(1979)) was obtained and maintained on L media containing 20 μg/mL hematin (United States Biochemicals). The plasmid library was transformed into SASX38 by electroporation using the Bio-Rad Gene Pulser and the manufacturer's conditions. The electroporated cells were plated on L agar containing 100 μg/ml ampicillin at a density of approximately 500,000 transformants/10 cm plate. The cells were then incubated at 37° C. for 40 hours in low light and selected for the ability to grow without the addition of exogenous heme. Heme prototrophs were recovered at a frequency of 400/10⁷ from the pFL61 library. Sequence analysis of twenty-two complementing clones showed that nine are of the type designated “Protox-1,” the protox gene expected to express a chloroplastic protox enzyme.

The pFL61 library is a yeast expression library, with the Arabidopsis cDNAs inserted bidirectionally. These cDNAs can also be expressed in bacteria. The protox cDNAs apparently initiate at an in-frame ATG in the yeast PGK 3′ sequence approximately 10 amino acids 5′ to the Notl cloning site in the vector and are expressed either from the lacZ promoter 300 bp further upstream or from an undefined cryptic bacterial promoter. Because Protox-1 cDNAs that included significant portions of a chloroplast transit sequence inhibited the growth of the E. coli SASX38 strain, the clone with the least amount of chloroplast transit sequence attached was chosen for mutagenesis/herbicide selection experiments. This clone, pSLV19, contains only 17 amino acids of the putative chioroplast transit peptide, with the DNA sequence beginning at bp 151 of the Arabidopsis Protox-1 cDNA (SEQ ID NO:1).

The plasmid pSLV19 was transformed into the random mutagenesis strain XL1-Red (Stratagene, La Jolla, Calif.). The transformation was plated on L media containing 50 μg/ml ampicillin and incubated for 48 hours at 37° C. Lawns of transformed cells were scraped from the plates and plasmid DNA prepared using the Wizard Megaprep kit (Promega, Madison, Wis.). Plasmid DNA isolated from this mutator strain is predicted to contain approximately one random base change per 2000 nucleotides (see Greener et al., Strategies 7(2):32-34 (1994).

The mutated plasmid DNA was transformed into the hemG mutant SASX38 (Sasarman et al., J. Gen. Microbiol. 113:297 (1979) and plated on L media containing various concentrations of protox-inhibiting herbicide. The plates were incubated for 2 days at 37° C. Plasmid DNA was isolated from all colonies that grew in the presence of herbicide concentrations that effectively killed the wild type strain. The isolated DNA was then transformed into SASX38 and plated again on herbicide to ensure that the resistance observed was plasmid-borne. The protox coding sequence from plasmids passing this screen was excised by Notl digestion, recloned into an unmutagenized vector, and tested again for the ability to confer herbicide tolerance. The DNA sequence of protox cDNAs that conferred herbicide resistance was then determined and mutations identified by comparison with the wild type Arabidopsis Protox-1 sequence (SEQ ID NO:1).

A single coding sequence mutant was recovered from the first mutagenesis experiment. This mutant leads to enhanced herbicide “resistance” only by increasing growth rate. It contains a C to A mutation at nucleotide 197 in SEQ ID NO:1 in the truncated chloroplast transit sequence of pSLV19, converting an ACG codon for threonine to an MG codon for lysine at amino acid 56 of SEQ ID NO:2, and resulting in better complementation of the bacterial mutant. This plasmid also contains a silent coding sequence mutation at nucleotide 1059, with AGT (Ser) changing to AGC (Ser). This plasmid was designated pMut-1.

The pMut-1 plasmid was then transformed into the mutator XL1-Red strain as described above and the mutated DNA was isolated and plated on an herbicide concentration that is lethal to the unmutagenized pMut-1 protox gene. Herbicide tolerant colonies were isolated after two days at 37° C. and analyzed as described above. Multiple plasmids were shown to contain herbicide resistant protox coding sequences. Sequence analysis indicated that the resistant genes fell into two classes. One resistance mutation identified was a C to T change at nucleotide 689 in the Arabidopsis Protox-1 sequence set forth in SEQ ID NO:1. This change converts a GCT codon for alanine at amino acid 220 of SEQ ID NO:2 to a GTT codon for valine, and was designated pAraC-1 Val.

A second class of herbicide resistant mutant contains an A to G change at nucleotide 1307 in the Arabidopsis Protox-1 sequence. This change converts a TAC codon for tyrosine at amino acid 426 to a TGC codon for cysteine, and was designated pAraC-2Cys.

A third resistant mutant has a G to A change at nucleotide 691 in the Arabidopsis Protox-1 sequence. This mutation converts a GGT codon for glycine at amino acid 221 to an AGT codon for serine at the codon position adjacent to the mutation in pAraC-1. This plasmid was designated pAraC-3Ser.

Resistant mutant pAraC-2Cys, in the pMut-1 plasmid, was deposited on Nov. 14, 1994 under the designation pWDC-7 with the Agricultural Research Culture Collection and given the deposit designation NRRL #21339N.

### Example 11

Additional Herbicide-Resistant Codon Substitutions at Positions Identified in the Random Screen

The amino acids identified as herbicide resistance sites in the random screen are replaced by other amino acids and tested for function and for herbicide tolerance in the bacterial system. Oligonucleotide-directed mutagenesis of the Arabidopsis Protox-1 sequence is performed using the Transformer Site-Directed Mutagenesis Kit (Clontech, Palo Alto, Calif.). After amino acid changes are confirmed by sequence analysis, the mutated plasmids are transformed into SASX38 and plated on L-amp¹⁰⁰ media to test for function and on various concentrations of protox-inhibiting herbicide to test for tolerance.

This procedure is applied to the alanine codon at nucleotides 688-690 and to the tyrosine codon at nucleotides 1306-1308 of the Arabidopsis Protox-1 sequence (SEQ ID NO:1). The results demonstrate that the alanine codon at nucleotides 688-690 can be changed to a codon for valine (pAraC-1Val), threonine (pAraC-1Thr), leucine (pAraC-1Leu), cysteine (pAraC-1Cys), or isoleucine (pAraC-1lle) to yield an herbicide-resistant protox enzyme that retains function. The results further demonstrate that the tyrosine codon at nucleotides 1306-1308 can be changed to a codon for cysteine (pAraC-2Cys), isoleucine (pAraC-2lle), leucine (pAraC-2Leu), threonine (pAraC-2Thr), methionine (pAraC-2Met), valine (pAraC-2Val), or alanine (pAraC-2Ala) to yield an herbicide-resistant protox enzyme that retains function.

### Example 12

Isolation of Additional Mutations that Increase Enzyme Function and/or Herbicide Tolerance of Previously Identified Resistant Mutants

Plasmids containing herbicide resistant protox genes are transformed into the mutator strain XL1-Red and mutated DNA is isolated as described above. The mutated plasmids are transformed into SASX38 and the transformants are screened on herbicide concentrations sufficient to inhibit growth of the original “resistant” mutant. Tolerant colonies are isolated and the higher tolerance phenotype is verified as being coding sequence dependent as described above. The sequence of these mutants is determined and mutations identified by comparison to the progenitor sequence.

This procedure was applied to the pAraC-1Val mutant described above. The results demonstrate that the serine codon at amino acid 305 (SEQ ID NO:2) can be changed to a codon for leucine to yield an enzyme with higher tolerance to protox-inhibiting herbicides than the pAraC-1 Val mutant alone. This second site mutation is designated AraC305Leu. The same results are demonstrated for the threonine codon at amino acid 249, where a change to either isoleucine or to alanine leads to a more tolerant enzyme. These changes are designated AraC249lle and AraC249Ala, respectively.

The procedure was also applied to the pAraC-2Cys mutant described above. The results demonstrate that the proline codon at amino acid 118 (SEQ ID NO:2) can be changed to a codon for leucine to yield an enzyme with higher tolerance to protox-inhibiting herbicides than the pAraC-1Cys mutant alone. This mutation is designated AraC118Leu. The same results are demonstrated for the serine codon at amino acid 305, where a change to leucine leads to a more tolerant pAraC-2Cys enzyme. This change was also isolated with the pAraC-1Val mutant as described above and is designated AraC305Leu. Additional mutations that enhance the herbicide resistance of the pAraC-2Cys mutant include an asparagine to serine change at amino acid 425, designated AraC425Ser, and a tyrosine to cysteine at amino acid 498, designated AraC498Cys.

These changes are referred to as “second site” mutations, because they are-not sufficient to confer herbicide tolerance alone, but rather enhance the function and/or the herbicide tolerance of an already mutant enzyme. This does not preclude the possibility that other amino acid substitutions at these sites could suffice to produce an herbicide tolerant enzyme since exhaustive testing of all possible replacements has not been performed.

### Example 13

Combining Identified Resistance Mutations with Identified Second Site Mutations to Create Highly Functional/Highly Tolerant Protox Enzymes

The AraC305Leu mutation described above was found to enhance the function/herbicide resistance of both the AraC-1Val and the AraC-2Cys mutant plasmids. In an effort to test the general usefulness of this second site mutation, it was combined with the AraC-2Leu, AraC-2Val, and AraC-2lle mutations and tested for herbicide tolerance. In each case, the AraC305Leu change significantly increased the growth rate of the resistant protox mutant on protox-inhibiting herbicide. Combinations of the AraC-2lle resistant mutant with either the second site mutant AraC249lle or AraC118Leu also produced more highly tolerant mutant protox enzymes. The AraC249lle mutation demonstrates that a second site mutation identified as enhancing an AraC-1 mutant may also increase the resistance of an AraC-2 mutant. A three mutation plasmid containing AraC-2lle, AraC305Leu, and AraC249lle has also been shown to produce a highly functional, highly herbicide tolerant protox-1 enzyme.

### Example 14

Identification of Sites in the Maize Protox-1 Gene that can be Mutated to Give Herbicide Tolerance

The pMut-1 Arabidopsis Protox-1 plasmid described above is very effective when used in mutagenesis/screening experiments in that it gives a high frequency of genuine coding sequence mutants, as opposed to the frequent up-promoter mutants that are isolated when other plasmids are used. In an effort to create an efficient plasmid screening system for maize Protox-1, the maize cDNA was engineered into the pMut-1 vector in approximately the same sequence context as the Arabidopsis cDNA. Using standard methods of overlapping PCR fusion, the 5′ end of the pMut-1 Arabidopsis clone (including 17 amino acids of chloroplast transit peptide with one mis-sense mutation as described above) was fused to the maize Protox-1 cDNA sequence starting at amino acid number 14 (SEQ ID NO:6) of the maize sequence. The 3′ end of the maize cDNA was unchanged. NotI restriction sites were placed on both ends of this fusion, and the chimeric gene was cloned into the pFL61 plasmid backbone from pMut-1. Sequence analysis revealed a single nucleotide PCR-derived silent mutation that converts the ACG codon at nucleotides 745-747 (SEQ ID NO:5) to an ACT codon, both of which encode threonine. This chimeric Arab-maize Protox-1 plasmid is designated pMut-3.

The pMut-3 plasmid was transformed into the mutator XL1-Red strain as described above and the mutated DNA was isolated and plated on an herbicide concentration that was lethal to the unmutagenized pMut-3 maize protox gene. Herbicide tolerant colonies were isolated after two days at 37° C. and analyzed as described above. This analysis revealed multiple plasmids containing herbicide resistant protox coding sequences. Sequence analysis showed 5 single base changes that individually result in an herbicide tolerant maize Protox-1 enzyme. Three of these mutations correspond to amino acid changes previously shown to confer tolerance at the homologous position in the Arabidopsis Protox-1 gene. Two of the three are pMzC-1Val and pMzC-1Thr, converting the alanine (GCT) at amino acid 164 (SEQ ID NO:6) to either valine (GAT) or to threonine (ACT). This position corresponds to the pAraC-1 mutations described above. The third analogous change converts the glycine (GGT) at amino acid 165 to Serine (AGT), corresponding to the AraC-3Ser mutation described above. These results serve to validate the expectation that herbicide-tolerant mutations identified in one plant protox gene may also confer herbicide tolerance in an equivalent plant protox gene from another species.

Two of the mutations isolated from the maize Protox-1 screen result in amino acid changes at residues not previously identified as herbicide resistance sites. One change converts cysteine (TGC) to phenylalanine (TTC) at amino acid 159 of the maize Protox-1 sequence (SEQ ID NO:6). The second converts isoleucine (ATA) to threonine (ACA) at amino acid 419.

Additional amino acid substitutions were made and tested at three of the maize mutant sites. Tolerance was demonstrated when glycine 165 was changed to leucine or when cysteine 159 was changed to either leucine or to lysine. Tolerant enzymes were also created by changing isoleucine 419 to histidine, glycine, or asparagine.

Individual amino acid changes that produced highly herbicide tolerant Arabidopsis Protox-1 enzymes were engineered into the maize Protox-1 gene by site-directed mutagenesis as described above. Bacterial testing demonstrated that changing the alanine (GCT) at amino acid 164 (SEQ ID NO:6) to leucine (CTT) produced a highly tolerant maize enzyme. No mutation analogous to the AraC-2 site in Arabidopsis was isolated in the maize random screen. However, changing this site, tyrosine 370 in the maize enzyme (SEQ ID NO:6), to either isoleucine or methionine did produce an herbicide tolerant enzyme.

### Example 15

Identification of Sites in the Wheat Protox-1 Gene that can be Mutated to Give Herbicide Tolerance

To create an efficient plasmid screening system for wheat Protox-1, the wheat cDNA was engineered into the pMut-1 vector as described above for the maize cDNA. This chimeric Arab-wheat Protox-1 plasmid is designated pMut-4. The pMut-4 DNA was mutated and screened for herbicide tolerance as described above. This analysis revealed multiple plasmids containing herbicide resistant protox coding sequences. Sequence analysis showed 7 single base changes that individually result in an herbicide tolerant wheat Protox-1 enzyme. Four of these mutations correspond to amino acid changes previously shown to confer tolerance at the homologous position in the Arabidopsis and/or in the maize Protox-1 gene. Two convert the alanine (GCT) at amino acid 211 (SEQ ID NO:10) to either valine (GAT) or to threonine (ACT). This position corresponds to the pAraC-1 mutations described above. The third analogous change converts the glycine (GGT) at amino acid 212 to Serine (AGT), corresponding to the AraC-3Ser mutation described above. The fourth converts isoleucine (ATA) to threonine (ACA) at amino acid 466, corresponding to the Mz419Thr mutant from maize.

Three of the mutations isolated from the wheat Protox-1 screen result in amino acid changes at residues not previously identified as herbicide resistance sites. One change converts valine (GTT) to leucine (CTT) at amino acid 356 of the wheat Protox-1 sequence (SEQ ID NO:10). A second converts serine (TCT) to proline (CCT) at amino acid 421. The third converts valine (GTT) to alanine (GCT) at amino acid 502.

### Example 16

Identification of Sites in the Soybean Protox-1 Gene that can be Mutated to Give Herbicide Tolerance

To create an efficient plasmid screening system for soybean Protox-1, the soybean cDNA was engineered into the pMut-1 vector as described above for the maize cDNA. This chimeric Arab-soybean Protox-1 plasmid is designated pMut-5. The pMut-5 DNA was mutated and screened for herbicide tolerance as described above. This analysis revealed multiple plasmids containing herbicide resistant protox coding sequences. Sequence analysis showed 4 single base changes that individually result in an herbicide tolerant soybean Protox-1 enzyme. Two of these mutations correspond to amino acid changes previously shown to confer tolerance at the homologous position in the Arabidopsis and/or in the wheat Protox-1 gene. One converts the alanine (GCA) at amino acid 226 (SEQ ID NO:12) to threonine (ACA). This position corresponds to the pAraC-1Thr mutation described above. The second analogous change converts the valine (GTT) at amino acid 517 to alanine (GCT), corresponding to the Wht502Val mutation from wheat.

Two of the mutations isolated from the soybean Protox-1 screen result in amino acid changes at a residue not previously identified as an herbicide resistance site. One change converts proline (CCT) to serine (TCT) at amino acid 369 of the soybean Protox-1 sequence (SEQ ID NO:12). A second converts this same proline369 to histidine (CAT).

Individual amino acid changes that produced highly herbicide tolerant Arabidopsis Protox-1 enzymes were engineered into the soybean Protox-1 gene by site directed mutagenesis as described above. Bacterial testing demonstrated that changing the alanine (GCA) at amino acid 226 (SEQ ID NO:12) to leucine produced a tolerant soybean enzyme. Changing the tyrosine (TAC) at amino acid 432 (SEQ ID NO:12) to either leucine or isoleucine also produced an herbicide tolerant enzyme.

### Example 17

Identification of Sites in the Sugar Beet Protox-1 Gene that can be Mutated to Give Herbicide Tolerance

To create an efficient plasmid screening system for sugar beet Protox-1, the sugar beet cDNA was engineered into the pMut-1 vector as described above for the maize cDNA. This chimeric Arab-sugar beet Protox-1 plasmid is designated pMut-6. The pMut-6 DNA was mutated and screened for herbicide tolerance as described above. This analysis revealed multiple plasmids containing herbicide resistant protox coding sequences. Sequence analysis showed a single base change that results in an herbicide tolerant sugar beet Protox-1 enzyme. This change converts tyrosine (TAC) at amino acid 449 to cysteine (TGC) and is analogous to the AraC-2 mutation in Arabidopsis.

Individual amino acid changes that produced highly herbicide tolerant Arabidopsis Protox-1 enzymes were engineered into the sugar beet Protox-1 gene by site directed mutagenesis as described above. Bacterial testing demonstrated that changing the tyrosine (TAC) at amino acid 449 to either leucine, isoleucine, valine, or methionine produced an herbicide tolerant sugar beet enzyme.

### Example 18

Identification of Sites in the Cotton Protox-1 Gene that can be Mutated to Give Herbicide Tolerance

In an effort to create an efficient plasmid screening system for cotton Protox-1, the cotton cDNA was engineered into the pMut-1 vector as described above for the maize cDNA. This chimeric Arab-cotton Protox-1 plasmid is designated pMut-7. The pMut-7 DNA was mutated and screened for herbicide tolerance as described above. This analysis revealed multiple plasmids containing herbicide resistant protox coding sequences. Sequence analysis showed 3 single base changes that individually result in an herbicide tolerant cotton Protox-1 enzyme. Two mutants change tyrosine (TAC) at amino acid 428 (SEQ ID NO:16) to cysteine (TGC) and to arginine (CGC), respectively. Arginine is a novel substitution giving tolerance at this previously identified AraC-2 site. The third mutation converts proline (CCC) to serine (TCC) at amino acid 365. This change corresponds to the soybean mutant Soy369Ser.

TABLE 3B
Cross Tolerance of Plant Protox Mutants to Various Protox Inhibitors

| | | | AraC-1Leu − | AraC-1Leu − | AraC-2Ile − | AraC-2Cys − | AraC-2Leu − | AraC-2Met − |
|---|---|---|---|---|---|---|---|---|
| | AraC- | AraC- | AraC- | AraC | AraC | AraC | AraC | |
| Formula | 1Leu | 2Ile | 2Met | 2Leu | 305Leu | 425Ser | 425Ser | 425Ser |
| XVII | + | + | + | + | + | + | + | + |
| VIIa | ++ | ++ | ++ | ++ | ++ | ++ | ++ | ++ |
| IV | ++ | − | + | ++ | + | − | + | + |
| XV | ++ | +++ | +++ | +++ | +++ | ++ | +++ | ++ |
| XI | ++ | ++ | ++ | ++ | ++ | ++ | ++ | ++ |
| XVI | +++ | +++ | +++ | +++ | +++ | + | ++ | ++ |
| XII | | | | | | | | |
| XIV | ++ | ++ | ++ | ++ | ++ | − | ++ | ++ |

### Section C: Expression of Herbicide-Resistant Protox Genes in Transgenic Plants

### Example 20

Engineering of Plants Tolerant to Protox-inhibiting Herbicides by Homologous Recombination or Gene Conversion

Because the described mutant coding sequences effectively confer herbicide tolerance when expressed under the control of the native protox promoter, targeted changes to the protox coding sequence in its native chromosomal location represent an alternative means for generating herbicide tolerant plants and plant cells. A fragment of protox DNA containing the desired mutations, but lacking its own expression signals (either promoter or 3′ untranslated region) can be introduced by any of several art-recognized methods (for instance, Agrobacterium transformation, direct gene transfer to protoplasts, microprojectile bombardment), and herbicide-tolerant transformants selected. The introduced DNA fragment also contains a diagnostic restriction enzyme site or other sequence polymorphism that is introduced by site-directed mutagenesis in vitro without changing the encoded amino acid sequence (i.e. a silent mutation). As has been previously reported for various selectable marker and herbicide tolerance genes (see, e.g., Paszkowski et al., EMBO J. 7: 4021-4026 (1988); Lee et al., Plant Cell 2: 415-425 (1990); Risseeuw et al., Plant J. 7: 109-119 (1995)). some transformants are found to result from homologous integration of the mutant DNA into the protox chromosomal locus, or from conversion of the native protox chromosomal sequence to the introduced mutant sequence. These transformants are recognized by the combination of their herbicide-tolerant phenotype, and the presence of the diagnostic restriction enzyme site in their protox chromosomal locus.

### Example 21

Construction of Plant Transformation Vectors

Numerous transformation vectors are available for plant transformation, and the genes of this invention can be used in conjunction with any such vectors. The selection of vector for use will depend upon the preferred transformation technique and the target species for transformation. For certain target species, different antibiotic or herbicide selection markers may be preferred. Selection markers used routinely in transformation include the nptII gene, which confers resistance to kanamycin and related antibiotics (Messing & Vierra, Gene 19: 259-268 (1982); Bevan et al., Nature 304:184-187 (1983)), the bar gene, which confers resistance to the herbicide phosphinothricin (White et al., Nucl Acids Res 18: 1062 (1990), Spencer et al. Theor Appl Genet 79: 625-631(1990)), the hph gene, which confers resistance to the antibiotic hygromycin (Blochinger & Diggelmann, Mol Cell Biol 4: 2929-2931), and the dhfr gene, which confers resistance to methotrexate (Bourouis et al., EMBO J. 2(7): 1099-1104 (1983)).

I. Construction of Vectors Suitable for Agrobacterium Transformation

Many vectors are available for transformation using Agrobacterium tumefaciens. These typically carry at least one T-DNA border sequence and include vectors such as pBIN19 (Bevan, Nucl. Acids Res. (1984)) and pXYZ. Below the construction of two typical vectors is described.

Construction of pCIB200 and pCIB2001: The binary vectors pCIB200 and pCIB2001 are used for the construction of recombinant vectors for use with Agrobacterium and was constructed in the following manner. pTJS75kan was created by Narl digestion of pTJS75 (Schmidhauser & Helinski, J Bacteriol. 164: 446-455 (1985)) allowing excision of the tetracycline-resistance gene, followed by insertion of an Accl fragment from pUC4K carrying an NPTII (Messing & Vierra, Gene 19: 259-268 (1982); Bevan et al., Nature 304: 184-187 (1983); McBride et aL., Plant Molecular Biology 14: 266-276 (1990)). Xhol linkers were ligated to the EcoRVfragment of pCIB7, which contains the left and right T-DNA borders, a plant selectable nos/nptII chimeric gene and the pUC polylinker (Rothstein et al., Gene 53: 153-161 (1987)), and the XhoI-digested fragment was cloned into SalI-digested pTJS75kan to create pCIB200 (see also EP 0 332 104, example 19). pCIB200 contains the following unique polylinker restriction sites: EcoRI, SstI, KpnI, BgllI, XbaI, and SalI. pCIB2001 is a derivative of pCIB200, which is created by the insertion into the polylinker of additional restriction sites. Unique restriction sites in the polylinker of pCIB2001 are EcoRI, SstI, KpnI, BgiII, XbaI, SalI, MluI, BciI, AvriI, ApaI, HpaI, and StuI. pCIB2001, in addition to containing these unique restriction sites also has plant and bacterial kanamycin selection, left and right T-DNA borders for Agrobacterium-mediated transformation, the RK2-derived trfA function for mobilization between E. coli and other hosts, and the OriTand OriV functions also from RK2. The pCIB2001 polylinker is suitable for the cloning of plant expression cassettes containing their own regulatory signals.

Construction of pCIB10 and Hygromycin Selection Derivatives Thereof: The binary vector pCIB10 contains a gene encoding kanamycin resistance for selection in plants, T-DNA right and left border sequences and incorporates sequences from the wide host-range plasmid pRK252 allowing it to replicate in both E. coil and Agrobacterium. Its construction is described by Rothstein et al., Gene 53: 153-161 (1987). Various derivatives of pCIB10 have been constructed that incorporate the gene for hygromycin B phosphotransferase described by Gritz et al., Gene 25: 179-188 (1983)). These derivatives enable selection of transgenic plant cells on hygromycin only (pCIB743), or hygromycin and kanamycin (pCIB715, pCIB717).

II. Construction of Vectors Suitable for non-Agrobacterium Transformation.

Transformation without the use of Agrobacterium tumefaciens circumvents the requirement for T-DNA sequences in the chosen transformation vector and consequently vectors lacking these sequences can be utilized in addition to vectors such as the ones described above that contain T-DNA sequences. Transformation techniques that do not rely on Agrobacterium include transformation via particle bombardment, protoplast uptake (e.g. PEG and electroporation) and microinjection. The choice of vector depends largely on the preferred selection for the species being transformed. Below, the construction of some typical vectors is described.

Construction of pCIB3064: pCIB3064 is a pUC-derived vector suitable for direct gene transfer techniques in combination with selection by the herbicide basta (or phosphinothricin). The plasmid pCIB246 comprises the CaMV 35S promoter in operational fusion to the E. coli GUS gene and the CaMV 35S transcriptional terminator and is described in the PCT published application WO 93/07278. The 35S promoter of this vector contains two ATG sequences 5′ of the start site. These sites were mutated using standard PCR techniques in such a way as to remove the ATG's and generate the restriction sites Sspi and PvuII. The new restriction sites were 96 and 37 bp away from the unique SalI site and 101 and 42 bp away from the actual start site. The resultant derivative of pCIB246 was designated pCIB3025. The GUS gene was then excised from pCIB3025 by digestion with SalI and SacI, the termini rendered blunt and religated to generate plasmid pCIB3060. The plasmid pJIT82 was obtained from the John Innes Centre, Norwich and the a 400 bp SmaI fragment containing the bar gene from Streptomyces viridochromogenes was excised and inserted into the HpaI site of pCIB3060 (Thompson et al. EMBO J 6: 2519-2523 (1987)). This generated pCIB3064, which comprises the bar gene under the control of the CaMV 35S promoter and terminator for herbicide selection, a gene for ampicillin resistance (for selection in E. coli) and a polylinker with the unique sites SphI, PstI, HindlII, and BamHI. This vector is suitable for the cloning of plant expression cassettes containing their own regulatory signals.

Construction of pSOG19 and pSOG35: pSOG35 is a transformation vector that utilizes the E. coli gene dihydrofolate reductase (DHFR) as a selectable marker conferring resistance to methotrexate. PCR was used to amplify the 35S promoter (˜800 bp), intron 6 from the maize AdhI gene (˜550 bp) and 18 bp of the GUS untranslated leader sequence from pSOG10. A 250 bp fragment encoding the E. coli dihydrofolate reductase type 11 gene was also amplified by PCR and these two PCR fragments were assembled with a SacI-PstI fragment from pBI221 (Clontech), which comprised the pUC19 vector backbone and the nopaline synthase terminator. Assembly of these fragments generated pSOG19, which contains the 35S promoter in fusion with the intron 6 sequence, the GUS leader, the DHFR gene and the nopaline synthase terminator. Replacement of the GUS leader in pSOG19 with the leader sequence from Maize Chlorotic Mottle Virus (MCMV) generated the vector pSOG35. pSOG19 and pSOG35 carry the pUC gene for ampicillin resistance and have HindlII, SphI, PstI and EcoRI sites available for the cloning of foreign sequences.

### Example 22

Construction of Plant Expression Cassettes

Gene sequences intended for expression in transgenic plants are firstly assembled in expression cassettes behind a suitable promoter and upstream of a suitable transcription terminator. These expression cassettes can then be easily transferred to the plant transformation vectors described above in Example 21.

I. Promoter Selection

The selection of a promoter used in expression cassettes will determine the spatial and temporal expression pattern of the transgene in the transgenic plant. Selected promoters will express transgenes in specific cell types (such as leaf epidermal cells, mesophyll cells, root cortex cells) or in specific tissues or organs (roots, leaves or flowers, for example) and this selection will reflect the desired location of expression of the transgene. Alternatively, the selected promoter may drive expression of the gene under a light-induced or other temporally regulated promoter. A further alternative is that the selected promoter be chemically regulated. This would provide the possibility of inducing expression of the transgene only when desired and caused by treatment with a chemical inducer.

II. Transcriptional Terminators

A variety of transcriptional terminators are available for use in expression cassettes. These are responsible for the termination of transcription beyond the transgene and its correct polyadenylation. Appropriate transcriptional terminators are those that are known to function in plants and include the CaMV 35S terminator, the tml terminator, the nopaline synthase terminator, the pea rbcS E9 terminator, as well as terminators naturally associated with the plant protox gene (i.e. “protox terminators”). These can be used in both monocotyledons and dicotyledons.

III. Sequences for the Enhancement or Regulation of Expression

Numerous sequences have been found to enhance gene expression from within the transcriptional unit and these sequences can be used in conjunction with the genes of this invention to increase their expression in transgenic plants.

Various intron sequences have been shown to enhance expression, particularly in monocotyledonous cells. For example, the introns of the maize AdhI gene have been found to significantly enhance the expression of the wild-type gene under its cognate promoter when introduced into maize cells. Intron 1 was found to be particularly effective and enhanced expression in fusion constructs with the chloramphenicol acetyltransferase gene (Callis et al., Genes Develop. 1: 1183-1200 (1987)). In the same experimental system, the intron from the maize bronze1 gene had a similar effect in enhancing expression (Callis et al., supra). Intron sequences have been routinely incorporated into plant transformation vectors, typically within the non-translated leader.

A number of non-translated leader sequences derived from viruses are also known to enhance expression, and these are particularly effective in dicotyledonous cells. Specifically, leader sequences from Tobacco Mosaic Virus (TMV, the SW-sequence”), Maize Chlorotic Mottle Virus (MCMV), and Alfalfa Mosaic Virus (AMV) have been shown to be effective in enhancing expression (e.g. Gallie et al. Nucl. Acids Res. 15: 8693-8711 (1987); Skuzeski et al. Plant Molec. Biol. 15: 65-79 (1990))

IV. Targeting of the Gene Product Within the Cell

Various mechanisms for targeting gene products are known to exist in plants and the sequences controlling the functioning of these mechanisms have been characterized in some detail. For example, the targeting of gene products to the chloroplast is controlled by a signal sequence that is found at the amino terminal end of various proteins and that is cleaved during chloroplast import yielding the mature protein (e.g. Comai et al. J. Biol. Chem. 263: 15104-15109 (1988)). These signal sequences can be fused to heterologous gene products to effect the import of heterologous products into the chloroplast (van den Broeck et al. Nature 313: 358-363 (1985)). DNA encoding for appropriate signal sequences can be isolated from the 5′ end of the cDNAs encoding the RUBISCO protein, the CAB protein, the EPSP synthase enzyme, the GS2 protein and many other proteins that are known to be chloroplast localized.

Other gene products are localized to other organelles such as the mitochondrion and the peroxisome (e.g. Unger et al. Plant Molec. Biol. 13: 411-418 (1989)). The cDNAs encoding these products can also be manipulated to effect the targeting of heterologous gene products to these organelles. Examples of such sequences are the nuclear-encoded ATPases and specific aspartate amino transferase isoforms for mitochondria. Targeting to cellular protein bodies has been described by Rogers et al., Proc. Natl. Acad. Sci. USA 82: 6512-6516 (1985)).

In addition, sequences have been characterized that cause the targeting of gene products to other cell compartments. Amino terminal sequences are responsible for targeting to the ER, the apoplast, and extracellular secretion from aleurone cells (Koehler & Ho, Plant Cell 2: 769-783 (1990)). Additionally, amino terminal sequences in conjunction with carboxy terminal sequences are responsible for vacuolar targeting of gene products (Shinshi et al., Plant Molec. Biol. 14: 357-368 (1990)).

By the fusion of the appropriate targeting sequences described above to transgene sequences of interest it is possible to direct the transgene product to any organelle or cell compartment. For chloroplast targeting, for example, the chloroplast signal sequence from the RUBISCO gene, the CAB gene, the EPSP synthase gene, or the GS2 gene is fused in frame to the amino terminal ATG of the transgene. The signal sequence selected should include the known cleavage site and the fusion constructed should take into account any amino acids after the cleavage site that are required for cleavage. In some cases this requirement may be fulfilled by the addition of a small number of amino acids between the cleavage site and the transgene ATG or alternatively replacement of some amino acids within the transgene sequence. Fusions constructed for chloroplast import can be tested for efficacy of chloroplast uptake by in vitro translation of in vitro transcribed constructions followed by in vitro chloroplast uptake using techniques described by (Bartlett et al. In: Edelmann et al. (Eds.) Methods in Chloroplast Molecular Biology, Elsevier. pp. 1081-1091 (1982); Wasmann et al. Mol. Gen. Genet. 205: 446-453 (1986)). These construction techniques are well known in the art and are equally applicable to mitochondria and peroxisomes. The choice of targeting that may be required for expression of the transgenes will depend on the cellular localization of the precursor required as the starting point for a given pathway. This will usually be cytosolic or chloroplastic, although it may is some cases be mitochondrial or peroxisomal. The products of transgene expression will not normally require targeting to the ER, the apoplast or the vacuole.

The above described mechanisms for cellular targeting can be utilized not only in conjunction with their cognate promoters, but also in conjunction with heterologous promoters so as to effect a specific cell targeting goal under the transcriptional regulation of a promoter that has an expression pattern different to that of the promoter from which the targeting signal derives.

### Example 23

Transformation of Dicotyledons

Transformation techniques for dicotyledons are well known in the art and include Agrobacterium-based techniques and techniques that do not require Agrobacterium. Non-Agrobacterium techniques involve the uptake of exogenous genetic material directly by protoplasts or cells. This can be accomplished by PEG or electroporation mediated uptake, particle bombardment-mediated delivery, or microinjection. Examples of these techniques are described by Paszkowski et al., EMBO J 3: 2717-2722 (1984), Potrykus et al., Mol. Gen. Genet. 199: 169-177 (1985), Reich et al., Biotechnology 4: 1001-1004 (1986), and Klein et al., Nature 327: 70-73 (1987). In each case the transformed cells are regenerated to whole plants using standard techniques known in the art.

Agrobacterium-mediated transformation is a preferred technique for transformation of dicotyledons because of its high efficiency of transformation and its broad utility with many different species. The many crop species that are routinely transformable by Agrobacterium include tobacco, tomato, sunflower, cotton, oilseed rape, potato, soybean, alfalfa and poplar (EP 0 317 511 (cotton), EP 0 249 432 (tomato, to Calgene), WO 87/07299 (Brassica, to Calgene), U.S. Pat. No. 4,795,855 (poplar)).

Transformation of the target plant species by recombinant Agrobacterium usually involves co-cultivation of the Agrobacterium with explants from the plant and follows protocols well known in the art. Transformed tissue is regenerated on selectable medium carrying the antibiotic or herbicide resistance marker present between the binary plasmid T-DNA borders.

### Example 24

Transformation of Monocotyledons

Transformation of most monocotyledon species has now also become routine. Preferred techniques include direct gene transfer into protoplasts using PEG or electroporation techniques, and particle bombardment into callus tissue. Transformations can be undertaken with a single DNA species or multiple DNA species (i.e. co-transformation) and both these techniques are suitable for use with this invention. Co-transformation may have the advantage of avoiding complex vector construction and of generating transgenic plants with unlinked loci for the gene of interest and the selectable marker, enabling the removal of the selectable marker in subsequent generations, should this be regarded desirable. However, a disadvantage of the use of co-transformation is the less than 100% frequency with which separate DNA species are integrated into the genome (Schocher et al. Biotechnology 4:1093-1096 (1986)).

Patent Applications EP 0 292,435 (to Ciba-Geigy), EP 0 392 225 (to Ciba-Geigy) and WO 93/07278 (to Ciba-Geigy) describe techniques for the preparation of callus and protoplasts from an elite inbred line of maize, transformation of protoplasts using PEG or electroporation, and the regeneration of maize plants from transformed protoplasts. Gordon-Kamm et al., Plant Cell 2: 603-618 (1990)) and Fromm et al., Biotechnology 8: 833-839 (1990)) have published techniques for transformation of A188-derived maize line using particle bombardment. Furthermore, application WO 93/07278 (to Ciba-Geigy) and Koziel et al., Biotechnology 11: 194-200 (1993)) describe techniques for the transformation of elite inbred lines of maize by particle bombardment. This technique utilizes immature maize embryos of 1.5-2.5 mm length excised from a maize ear 14-15 days after pollination and a PDS-1000He Biolistics device for bombardment.

Transformation of rice can also be undertaken by direct gene transfer techniques utilizing protoplasts or particle bombardment. Protoplast-mediated transformation has been described for Japonica-types and Indica-types (Zhang et al., Plant Cell Rep 7: 379-384 (1988); Shimamoto et al. Nature 338: 274-277 (1989); Datta et al. Biotechnology 8: 736-740 (1990)). Both types are also routinely transformable using particle bombardment (Christou et al. Biotechnology 9: 957-962 (1991)).

Patent Application EP 0 332 581 (to Ciba-Geigy) describes techniques for the generation, transformation and regeneration of Pooideae protoplasts. These techniques allow the transformation of Dactylis and wheat. Furthermore, wheat transformation was been described by Vasil et al., Biotechnology 10: 667-674 (1992)) using particle bombardment into cells of type C long-term regenerable callus, and also by Vasil et al., Biotechnology 11: 1553-1558 (1993)) and Weeks et al., Plant Physiol. 102: 1077-1084 (1993) using particle bombardment of immature embryos and immature embryo-derived callus. A preferred technique for wheat transformation, however, involves the transformation of wheat by particle bombardment of immature embryos and includes either a high sucrose or a high maltose step prior to gene delivery. Prior to bombardment, any number of embryos (0.75-1 mm in length) are plated onto MS medium with 3% sucrose (Murashige & Skoog, Physiologia Plantarum 15: 473-497 (1962)) and 3 mg/l 2,4-D for induction of somatic embryos, which is allowed to proceed in the dark. On the chosen day of bombardment, embryos are removed from the induction medium and placed onto the osmoticum (i.e. induction medium with sucrose or maltose added at the desired concentration, typically 15%). The embryos are allowed to plasmolyze for 2-3 h and are then bombarded. Twenty embryos per target plate is typical, although not critical. An appropriate gene-carrying plasmid (such as pCIB3064 or pSG35) is precipitated onto micrometer size gold particles using standard procedures. Each plate of embryos is shot with the DuPont Biolistics, helium device using a burst pressure of ˜1000 psi using a standard 80 mesh screen. After bombardment, the embryos are placed back into the dark to recover for about 24 h (still on osmoticum). After 24 hrs, the embryos are removed from the osmoticum and placed back onto induction medium where they stay for about a month before regeneration. Approximately one month later the embryo explants with developing embryogenic callus are transferred to regeneration medium (MS +1 mg/liter NAA, 5 mg/liter GA), further containing the appropriate selection agent (10 mg/l basta in the case of pCIB3064 and 2 mg/L methotrexate in the case of pSOG35). After approximately one month, developed shoots are transferred to larger sterile containers known as “GA7s” that contained half-strength MS, 2% sucrose, and the same concentration of selection agent. Patent application WO 94/13822 describes methods for wheat transformation and is hereby incorporated by reference.

### Example 25

Isolation of the Arabidopsis thaliana Protox-1 Promoter Sequence

A Lambda Zap II genomic DNA library prepared from Arabidopsis thaliana (Columbia, whole plant) was purchased from Stratagene. Approximately 125,000 phage were plated at a density of 25,000 pfu per 15 cm Petri dish and duplicate lifts were made onto Colony/Plaque Screen membranes (NEN Dupont). The plaque lifts were probed with the Arabidopsis Protox-1 cDNA (SEQ ID NO:1 labeled with 32P-dCTP by the random priming method (Life Technologies). Hybridization and wash conditions were at 65° C. as described in Church and Gilbert, Proc. Natl. Acad. Sci. USA 81: 1991-1995 (1984). Positively hybridizing plaques were purified and in vivo excised into pBluescript plasmids. Sequence from the genomic DNA inserts was determined by the chain termination method using dideoxy terminators labeled with fluorescent dyes (Applied Biosystems, Inc.). One clone, AraPT1 Pro, was determined to contain 580 bp of Arabidopsis sequence upstream from the initiating methionine (ATG) of the Protox-1 protein coding sequence. This clone also contains coding sequence and introns that extend to bp 1241 of the Protox-1 cDNA sequence. The 580 bp 5′ noncoding fragment is the putative Arabidopsis Protox-1 promoter, and the sequence is set forth in SEQ ID NO:13.

AraPT1 Pro was deposited December 15,1995, as pWDC-11 (NRRL #B-21515)

### Example 26

Construction of Plant Transformation Vectors Expressing Altered Protox-1 Genes Behind the Native Arabidopsis Protox-1 Promoter

A full-length cDNA of the appropriate altered Arabidopsis Protox-1 cDNA was isolated as an EcoRI-XhoI partial digest fragment and cloned into the plant expression vector pCGN1761 ENX (see Example 9 of International application No. PCT/IB95/00452 filed Jun. 8, 1995, published Dec. 21, 1995 as WO 95/34659). This plasmid was digested with NcoI and BamHI to produce a fragment comprised of the complete Protox-1 cDNA plus a transcription terminator from the 3′ untranslated sequence of the tml gene of Agrobacterium tumefaciens. The AraPT1 Pro plasmid described above was digested with NcoI and BamHI to produce a fragment comprised of pBluescript and the 580 bp putative Arabidopsis Protox-1 promoter. Ligation of these two fragments produced a fusion of the altered protox cDNA to the native protox promoter. The expression cassette containing the Protox-1 promoter/Protox-1 cDNA/tml terminator fusion was excised by digestion with KpnI and cloned into the binary vector pCIB200. The binary plasmid was transformed by electroporation into Agrobacterium and then into Arabidopsis using the vacuum infiltration method (Bechtold et al., C.R. Acad. Sci. Paris 316: 1194-1199 (1993). Transformants expressing altered protox genes were selected on kanamycin or on various concentrations of protox inhibiting herbicide.

### Example 27

Production of Herbicide Tolerant Plants by Expression of a Native Protox-1 Promoter/Altered Protox-1 Fusion

Using the procedure described above, an Arabidopsis Protox-1 cDNA containing a TAC to ATG (Tyrosine to Methionine) change at nucleotides 1306-1308 in the Protox-1 sequence (SEQ ID NO:1) was fused to the native Protox-1 promoter fragment and transformed into Arabidopsis thaliana . This altered Protox-1 enzyme (AraC-2Met) has been shown to be >10-fold more tolerant to various protox-inhibiting herbicides than the naturally occurring enzyme when tested in the previously described bacterial expression system. Seed from the vacuum infiltrated plants was collected and plated on a range (10.0 nM-1.0 uM) of a protox inhibitory aryluracil herbicide of formula XVII. Multiple experiments with wild type Arabidopsis have shown that a 10.0 nM concentration of this compound is sufficient to prevent normal seedling germination. Transgenic seeds expressing the AraC-2Met altered enzyme fused to the native Protox-1 promoter produced normal Arabidopsis seedlings at herbicide concentrations up to 500 nM, indicating at least 50-fold higher herbicide tolerance when compared to wild-type Arabidopsis. This promoter/altered protox enzyme fusion therefore functions as an effective selectable marker for plant transformation. Several of the plants that germinated on 100.0 nM of protox-inhibiting herbicide were transplanted to soil, grown 2-3 weeks, and tested in a spray assay with various concentrations of the protox-inhibiting herbicide. When compared to empty vector control transformants, the AraPT1 Pro/AraC-2Met transgenics were>10-fold more tolerant to the herbicide spray.

### Example 28

Demonstration of resistant mutations' cross-tolerance to various protox-inhibiting compounds in an Arabidopsis germination assay.

Using the procedure described above, an Arabidopsis Protox-1 cDNA containing both a TAC to ATC (tyrosine to isoleucine) change at nucleotides 1306-1308 and a TCA to TTA (serine to leucine) change at nucleotides 945-947 in the Protox-1 sequence (SEQ ID NO:1) was fused to the native Protox-1 promoter fragment and transformed into Arabidopsis thaliana. This altered Protox-1 enzyme (AraC-211e +AraC305Leu) has been shown to be>10-fold more tolerant to a protox inhibitory aryluracil herbicide of formula XVII than the naturally occurring enzyme when tested in a bacterial system (see Examples 9-13). Homozygous Arabidopsis lines containing this fusion were generated from transformants that showed high tolerance to a protox inhibiting herbicide in a seedling germination assay as described above. The seed from one line was tested for cross-tolerance to various protox-inhibitory compounds by repeating the germination assay on concentrations of the compounds that had been shown to inhibit germination of wild-type Arabidopsis. The results from these experiments are shown in Table 4.

TABLE 4
Cross Tolerance to Various Protox Inhibitors
in a Seed Germination Assay

| Formula | Common name | Tolerance |
|---|---|---|
| II | acifluorofen | + |
| III | fomasafen | + |
| IV | fluoroglycofen | ± |
| IVb | bifenox | + |
| IVc | oxyfluorofen | + |
| IVd | lactofen | ± |
| VIIa | fluthiacet-methyl | ++ |
| X | sulfentrazone | + |
| XI | flupropazil | ++ |
| XIV | flumiclorac | + |
| XVI | flumioxazin | +++ |
| XVII | | ++ |
| XXIa | BAY 11340 | + |
| XXII | | ++ |

± ≦10× more tolerant than wt
+ ≧10× more tolerant than wt
++ ≧100× more tolerant than wt
+++ ≧1000× more tolerant than wt

+≦10× more tolerant than wt

+≧10× more tolerant than wt

++≧100× more tolerant than wt

+++≧1000× more tolerant than wt

### Example 29

Isolation of a Maize Protox-1 Promoter Sequence

A Zea Mays (Missouri 17 inbred, etiolated seedlings) genomic DNA library in the Lambda FIX II vector was purchased from Stratagene. Approximately 250,000 pfu of the library was plated at a density of 50,000 phage per 15 cm plate and duplicate lifts were made onto Colony/Plaque screen membranes (NEN Dupont). The plaque lifts were probed with the maize Protox-1 cDNA (SEQ ID NO:5) labeled with 32P-dCTP by the random priming method (Life Technologies). Hybridization and wash conditions were at 65° C. as described in Church and Gilbert, Proc. Natl. Acad. Sci. USA 81:1991-1995 (1984). Lambda phage DNA was isolated from three positively hybridizing phage using the Wizard Lambda Preps DNA Purification System (Promega). Analysis by restriction digest, hybridization patterns, and DNA sequence analysis identified a lambda clone containing approximately 3.5 kb of maize genomic DNA located 5′ to the maize Protox-1 coding sequence previously isolated as a cDNA clone. This fragment includes the maize Protox-1 promoter. The sequence of this fragment is set forth in SEQ ID NO:14. From nucleotide 1 to 3532, this sequence is comprised of 5′ noncoding sequence. From nucleotide 3533 to 3848, this sequence encodes the 5′ end of the maize Protox-1 protein.

A plasmid containing the sequence of SEQ ID NO:14 fused to the remainder of the maize Protox-1 coding sequence was deposited Mar. 19, 1996 as pWDC-14 (NRRL #B-21546).

### Example 30

Construction of Plant Transformation Vectors Expressing Altered Protox-1 Genes Behind the Native Maize Protox-1 Promoter

The 3848 bp maize genomic fragment (SEQ ID NO:14) was excised from the isolated lambda phage clone as a SalI-KpnI partial digest product and ligated to a KpnI-NotI fragment derived from an altered maize Protox-1 cDNA that contained an alanine to leucine change at amino acid 164 (SEQ ID NO:6). This created a fusion of the native maize Protox-1 promoter to a full length cDNA that had been shown to confer herbicide tolerance in a bacterial system (Examples 9-14). This fusion was cloned into a pUC18 derived vector containing the CaMV 35S terminator sequence to create a protox promoter/altered protox cDNA/terminator cassette. The plasmid containing this cassette was designated pWCo-1.

A second construct for maize transformation was created by engineering the first intron found in the coding sequence from the maize genomic clone back into the maize cDNA. The insertion was made using standard overlapping PCR tusion techniques. The intron (SEQ ID NO:25) was 93 bp long and was inserted between nucleotides 203 and 204 of SEQ ID NO:6, exactly as it appeared in natural context in the lambda clone described in Example 29. This intron-containing version of the expression cassette was designated pWCo-2.

Example 31

Demonstration of Maize Protox-1 Promoter Activity in Transgenic Maize Plants

Maize plants transformed with maize protox promoter/altered protox fusions were identified using PCR analysis with primers specific for the transgene. Total RNA was prepared from the PCR positive plants and reverse-transcribed using Superscript M-MLV (Life Technologies) under recommended conditions. Two microliters of the reverse transcription reaction was used in a PCR reaction designed to be specific for the altered protox sequence. While untransformed controls give no product in this reaction, approximately 85% of plants transformed with pWCo-1 gave a positive result, indicating the presence of mRNA derived from the transgene. This demonstrates some level of activity for the maize protox promoter. The RNA's from the transgenic maize plants were also subjected to standard northern blot analysis using the radiolabeled maize protox cDNA fragment from SEQ ID NO:6 as a probe. Protox-1 mRNA levels significantly above those of untransformed controls were detected in some of the transgenic maize plants. This elevated mRNA level is presumed to be due to expression of altered protox-1 mRNA from the cloned maize protox promoter.

### Example 32

Isolation of a Sugar Beet Protox-1 Promoter Sequence

A genomic sugar beet library was prepared by Stratagene in the Lambda Fix II vector. Approximately 300,000 pfu of the library was plated and probed with the sugar beet protox-1 cDNA sequence (SEQ ID NO:17) as described for maize in Example 29. Analysis by restriction digest, hybridization patterns and DNA sequence analysis identified a lambda clone containing approximately 7 kb of sugar beet genomic DNA located 5′ to the sugar beet coding sequence previously isolated as a cDNA clone. A PstI-SalI fragment of 2606 bb was subcloned from the lambda clone into a pBluescript vector. This fragment contains 2068 bp of 5′ noncoding sequence and includes the sugar beet protox-1 promoter sequence. It also includes the first 453 bp of the protox-1 coding sequence and the 85 bp first intron contained in the coding sequence. The sequence of this fragment is set forth in SEQ ID NO:26.

A plasmid containing the sequence of SEQ ID NO:26 was deposited Dec. 6, 1996 as pWDC-20 (NRRL #B-21650).

### Example 33

Construction of Plant Transformation Vectors Expressing Altered Sugar Beet Protox-1 Genes Behind the Native Sugar Beet Protox-1 Promoter

The sugar beet genomic fragment (SEQ ID NO:26) was excised from the genomic subclone described in Example 32 as a SacI-BsrGI fragment that includes 2068 bp of 5′ noncoding sequence and the first 300 bp of the sugar beet Protox-1 coding sequence. This fragment was ligated to a BsrGI-NotI fragment derived from an altered sugar beet Protox-1 cDNA that contained a tyrosine to methionine change at amino acid 449 (SEQ ID NO:18). This created a fusion of the native sugar beet Protox-1 promoter to a full length cDNA that had been shown to confer herbicide tolerance in a bacterial system (Examples 9-14). This fusion was cloned into a pUC18 derived vector containing the CaMV 35S terminator sequence to create a protox promoter/altered protox cDNA/terminator cassette. The plasmid containing this cassette was designated pWCo-3.

### Example 34

Production of Herbicide Tolerant Plants by Expression of a Native Sugar Beet Protox-1 Promoter/Altered Sugar Beet Protox-1 Fusion

The expression cassette from pWCo-3 is transformed into sugar beet using any of the transformation methods applicable to dicot plants, including Agrobacterium, protoplast, and biolistic transformation techniques. Transgenic sugar beets expressing the altered protox-1 enzyme are identified by RNA-PCR and tested for tolerance to protox-inhibiting herbicides at concentrations that are lethal to untransformed sugar beets.

### Section D: Expression of Protox Genes in Plant Plastids

### Example 35

Preparation of a Chimeric Gene Containing the Tobacco Plastid clpP Gene Promoter and Native clpP 5′ Untranslated Sequence Fused to a GUS Reporter Gene and Plastid rps16 Gene 3′ Untranslated Sequence in a Plastid Transformation Vector

I. Amplification of the Tobacco Plastid clpP Gene Promoter and Complete 5′ Untranslated RNA (5′ UTR).

Total DNA from N. tabacum c.v. “Xanthi NC” was used as the template for PCR with a left-to-right “top strand” primer comprising an introduced EcoRI restriction site at position—197 relative to the ATG start codon of the constitutively expressed plastid clpP gene (primer Pclp_P1a: 5′-GCGGAATTCATACTTATTTATCATTAGAAAG-3′ (SEQ ID NO:27); EcoRI restriction site underlined) and a right-to-left “bottom strand” primer homologous to the region from −21 to −1 relative to the ATG start codon of the clpP promoter that incorporates an introduced NcoI restriction site at the start of translation (primer Pclp_{P—}2b: 5′-GCGCCATGGTAAATGAAAGAAAGAACTAAA-3′ (SEQ ID NO:28); NcoI restriction site underlined). This PCR reaction was undertaken with Pfu thermostable DNA polymerase (Stratagene, La Jolla Calif.) in a Perkin Elmer Thermal Cycler 480 according to the manufacturer's recommendations (Perkin Elmer/Roche, Branchburg, N.J.) as follows: 7 min 95° C., followed by 4 cycles of 1 min 95° C./2 min 43° C./1 min 72° C., then 25 cycles of 1 min 95° C./2 min 55° C./1 min 72° C. The 213 bp amplification product comprising the promoter and 5′ untranslated region of the clpP gene containing an EcoRI site at its left end and an NcoI site at its right end and corresponding to nucleotides 74700 to 74505 of the N. tabacum plastid DNA sequence (Shinozaki et al., EMBO J. 5: 2043-2049 (1986)) was gel purified using standard procedures and digested with EcoRI and NcoI (all restriction enzymes were purchased from New England Biolabs, Beverly, Mass.).

II. Amplification of the Tobacco Plastid rps16 Gene 3′ Untranslated RNA Sequence (3′UTR).

Total DNA from N. tabacum c.v. “Xanthi NC” was used as the template for PCR as described above with a left-to-right “top strand” primer comprising an introduced XbaI restriction site immediately following the TAA stop codon of the plastid rps16 gene encoding ribosomal protein S16 (primer rps16P_{—}1a (5′-GCGTCTAGATCAACCGMATTCAATTAAGG-3′ (SEQ ID NO:30); XbaI restriction site underlined) and a right-to-left “bottom strand” primer homologous to the region from +134 to +151 relative to the TAA stop codon of rps16 that incorporates an introduced HindlII restriction site at the 3′ end of the rps16 3′ UTR (primer rps16P_{—}1b (5′-CGCAAGCTTCAATGGAAGCMTGATM-3′ (SEQ ID NO:31); HindlII restriction site underlined). The 169 bp amplification product comprising the 3′ untranslated region of the rps16 gene containing an XbaI site at its left end and a HindlII site at its right end and containing the region corresponding to nucleotides 4943 to 5093 of the N. tabacum plastid DNA sequence (Shinozaki et al., 1986) was gel purified and digested with XbaI and HindlII.

III. Ligation of a GUS Reporter Gene Fragment to the cIpP Gene Promoter and 5′ and 3′ UTR's.

An 1864 bp β-glucuronidase (GUS) reporter gene fragment derived from plasmid pRAJ275 (Clontech) containing an NcoI restriction site at the ATG start codon and an XbaI site following the native 3′ UTR was produced by digestion with NcoI and XbaI. This fragment was ligated in a four-way reaction to the 201 bp EcoRI/NcoI clpP promoter fragment, the 157 bp XbaI/HindlII rps16 3′UTR fragment, and a 3148 bp EcoRI/HindlII fragment from cloning vector pGEM3Zf(−) (Promega, Madison Wis.) to construct plasmid pPH138. Plastid transformation vector pPH140 was constructed by digesting plasmid pPRV111a (Zoubenko et al. 1994) with EcoRi and HindlII and ligating the resulting 7287 bp fragment to a 2222 bp EcoRI/HindlII fragment of pPH138.

### Example 36

Preparation of a Chimeric Gene Containing the Tobacco Plastid cIpP Gene Promoter Plus Tobacco Plastid psbA Gene Minimal 5′ Untranslated Sequence Fused to a GUS Reporter Gene and Plastid rps16 Gene 3′ Untranslated Sequence in a Plastid Transformation Vector

Amplification of the tobacco plastid clpP gene promoter and truncated 5′ untranslated RNA (5′ UTR): Total DNA from N. tabacum c.v. “Xanthi NC” was used as the template for PCR as described above with the left-to-right “top strand” primer Pclp_P1a (SEQ ID NO:27) and a right-to-left “bottom strand” primer homologous to the region from −34 to −11 relative to the ATG start codon of the clpP promoter that incorporates an introduced XbaI restriction site in the clpP 5′ UTR at position −11 (primer Pclp_P1 b: 5′-GCGTCTAGAAAGMCTAAATACTATATTTCAC-3′ (SEQ ID NO:29); XbaI restriction site underlined). The 202 bp amplification product comprising the promoter and truncated 5′ UTR of the clpP gene containing an EcoRI site at its left end and an XbaI site at its right end was gel purified and digested with XbaI. The XbaI site was subsequently filled in with Kienow DNA polymerase (New England Biolabs) and the fragment digested with EcoRI. This was ligated in a five-way reaction to a double stranded DNA fragment corresponding to the final 38 nucleotides and ATG start codon of the tobacco plastid psbA gene 5′ UTR (with an NcoI restriction site overhang introduced into the ATG start codon) that was created by annealing the synthetic oligonucleotides minpsb_U (top strand: 5′-GGGAGTCCCTGATGATTAAATAAACCMGATTTTAC-3′ (SEQ ID NO:32)) and minpsb_L (bottom strand: 5′-CATGGTAAAATCTTGGTTTATTTAATCATCAGGGACTCCC-3′ (SEQ ID NO:33); NcoI restriction site 5′ overhang underlined), the NcolI/XbaI GUS reporter gene fragment described above, the XbaI/HindlII rps16 3′UTR fragment described above, and the EcoRI/HindlII pGEM3Zf(−) fragment described above to construct plasmid pPH139. Plastid transformation vector pPH144 was constructed by digesting plasmid pPRV111a (Zoubenko, et al., Nucleic Acids Res 22: 3819-3824 (1994)) with EcoRI and HindlII and ligating the resulting 7287 bp fragment to a 2251 bp EcoRI/HindlII fragment of pPH139.

### Example 37

Preparation of a Chimeric Gene Containing the Tobacco Plastid clpP Gene Promoter and Complete 5′ Untranslated Sequence Fused to the Arabidopsis thaliana Protox-1 Coding Sequence and Plastid rps16 Gene 3′ Untranslated Sequence in a Vector for Tobacco Plastid Transformation

Miniprep DNA from plasmid AraC-2Met carrying an Arabidopsis thaliana NotI insert that includes cDNA sequences from the Protoporphyrinogen IX Oxidase (“PROTOX”) gene encoding a portion of the amino terminal plastid transit peptide, the full-length cDNA and a portion of the 3′ untranslated region was used as the template for PCR as described above using a left-to-right “top strand” primer (with homology to nucleotides +172 to +194 relative to the ATG start codon of the full length precursor protein) comprising an introduced NcoI restriction site and new ATG start codon at the deduced start of the mature PROTOX protein coding sequence (primer APRTXP1a: 5′-GGGACCATGGATTGTGTGATTGTCGGCGGAGG-3′ (SEQ ID NO:34); NcoI restriction site underlined) and a right-to-left “bottom strand” primer homologous to nucleotides +917 to +940 relative to the native ATG start codon of the PROTOX precursor protein (primer APRTXP1b: 5′-CTCCGCTCTCCAGCTTAGTGATAC-3′ (SEQ ID NO:35)). The 778 bp product was digested with NcoI and SfuI and the resulting 682 bp fragment ligated to an 844 bp SfuI/NotI DNA fragment of AraC-2Met comprising the 3′ portion of the PROTOX coding sequence and a 2978 bp NcoI/NotI fragment of the cloning vector pGEM5Zf(+) (Promega, Madison Wis.) to construct plasmid pPH141. Plastid transformation vector pPH143 containing the clpP promoter driving the Formula XVII-resistant AraC-2Met PROTOX gene with the rps16 3′ UTR was constructed by digesting pPH141 with NcoI and SspI and isolating the 1491 bp fragment containing the complete PROTOX coding sequence, digesting the rps16P_{—}1a and rps16P_{—}1b PCR product described above with HindlII, and ligating these to a 7436 bp NcoI/HindlII fragment of pPH140.

### Example 38

Preparation of a Chimeric Gene Containing the Tobacco Plastid clpP Gene Promoter Plus Tobacco Plastid psbA Gene Minimal 5′ Untranslated Sequence Fused to the Arabidopsis thaliana Protox-1 Coding Sequence and Plastid rps16 Gene 3′ Untranslated Sequence in a Vector for Tobacco Plastid Transformation Plastid transformation vector pPH145 containing the clpP promoter/psbA 5′ UTR fusion driving the Formula XVII-resistant AraC-2Met PROTOX gene with the rps16 3′ UTR was constructed by digesting pPH141 with NcoI and SspI and isolating the 1491 bp fragment containing the complete PROTOX coding sequence, digesting the rps16P_{—}1a and rps16P_{—}1b PCR product described above with HindlII, and ligating these to a 7465 bp NcoI/HindlII fragment of pPH144.

### Example 39

Preparation of a Chimeric Gene Containing the Tobacco Plastid clpP Gene Promoter and 5′ Untranslated Sequence Fused to the EPSP Synthase Coding Sequence and Plastid rps16 Gene 3′ Untranslated Sequence in a Vector for Tobacco Plastid Transformation

A cDNA library is screened for the 5-enolpyruvyl-3-phosphoshikimate synthase (EPSP synthase) gene (U.S. Pat. Nos. 5,310,667, 5,312,910, and 5,633,435, all incorporated herein by reference). A plasmid clone containing the full length EPSP synthase gene cDNA is isolated by standard techniques of molecular cloning. PCR primers are designed for amplification of the mature-size EPSP synthase coding sequence from this plasmid using a top strand primer having a 5′ extension containing an NcoI restriction site inserted at amino acid −1 from the deduced mature protein start, thus creating an ATG start codon at this position, and a bottom strand primer having a 5′ extension containing an XbaI restriction site downstream of the stop codon of the EPSP mature coding sequence in the amplified PCR product. The PCR amplification is performed using the designated primers and plasmid DNA template according to standard protocols. Amplified products are cloned and sequenced and a NcoI-XbaI DNA fragment containing the complete mature EPSP synthase coding sequence is isolated by restriction digest with NcoI and XbaI, electrophoresis on a 0.8% TAE agarose gel, and phenol extraction of the excised band.

A plastid transformation vector containing the clpP promoter directing transcription of the mature-sized EPSP synthase gene with the rps16 3′ UTR is constructed by digesting pPH140 with NcoI and XbaI and purifying the fragment containing the vector backbone, 5′ and 3′ plastid integration targeting sequences, aadA selectable marker cassette, and clpP promoter/rps16 3′ UTR expression sequences. This product is ligated in a two-way reaction with the NcoI-XbaI DNA fragment containing the mature-sized EPSP synthase coding sequence isolated as described above.

### Example 40

Preparation of a Chimeric Gene Containing the Tobacco Plastid clpP Gene Promoter and 5′ Untranslated Sequence Fused to the ALS Coding Sequence and Plastid rps16 Gene 3′ Untranslated Sequence in a Vector for Tobacco Plastid Transformation

A cDNA library is screened for the acetolactate synthase (ALS) gene (U.S. Pat. No. 5,013,659). A plasmid clone containing the full length ALS gene cDNA is isolated by standard techniques of molecular cloning. PCR primers are designed for amplification of the mature-size ALS coding sequence from this plasmid using a top strand primer having a 5′ extension containing an NcoI restriction site inserted at amino acid −1 from the deduced mature protein start, thus creating an ATG start codon at this position, and a bottom strand primer having a 5′ extension containing an XbaI restriction site downstream of the stop codon of the ALS mature coding sequence in the amplified PCR product. The PCR amplification is performed using the designated primers and plasmid DNA template according to standard protocols. Amplified products are cloned and sequenced and a NcoI-XbaI DNA fragment containing the complete mature ALS coding sequence is isolated by restriction digest with NcoI and XbaI, electrophoresis on a 0.8% TAE agarose gel, and phenol extraction of the excised band.

A plastid transformation vector containing the clpP promoter driving the mature-sized ALS gene with the rps16 3′ UTR is constructed by digesting pPH140 with Ncol and Xbal and purifying the fragment containing the vector backbone, 5′ and 3′ plastid integration targeting sequences, aadA selectable marker cassette, and clpP promoter/rps16 3′ UTR expression sequences. This product is ligated in a two-way reaction with the NcoI-XbaI DNA fragment containing the mature-sized ALS coding sequence isolated as described above.

### Example 41

Preparation of a Chimeric Gene Containing the Tobacco Plastid clpP Gene Promoter and 5′ Untranslated Sequence Fused to the AHAS Coding Sequence and Plastid rps16 Gene 3′ Untranslated Sequence in a Vector for Tobacco Plastid Transformation

A cDNA library is screened for the acetohydroxyacid synthase (AHAS) gene (U.S. Pat. No. 4,761,373). A plasmid clone containing the full length AHAS gene cDNA is isolated by standard techniques of molecular cloning. PCR primers are designed for amplification of the mature-size AHAS coding sequence from this plasmid using a top strand primer having a 5′ extension containing an NcoI restriction site inserted at amino acid −1 from the deduced mature protein start, thus creating an ATG start codon at this position, and a bottom strand primer having a 5′ extension containing an XbaI restriction site downstream of the stop codon of the AHAS mature coding sequence in the amplified PCR product. The PCR amplification is performed using the designated primers and plasmid DNA template according to standard protocols. Amplified products are cloned and sequenced and a NcoI-XbaI DNA fragment containing the complete mature AHAS coding sequence is isolated by restriction digest with NcoI and XbaI, electrophoresis on a 0.8% TAE agarose gel, and phenol extraction of the excised band.

A plastid transformation vector containing the clpP promoter driving the mature-sized AHAS gene with the rps16 3′ UTR is constructed by digesting pPH140 with NcoI and XbaI and purifying the fragment containing the vector backbone, 5′ and 3′ plastid integration targeting sequences, aadA selectable marker cassette, and clpP promoter/rps16 3′ UTR expression sequences. This product is ligated in a two-way reaction with the NcoI-XbaI DNA fragment containing the mature-sized AHAS coding sequence isolated as described above.

### Example 42

Preparation of a Chimeric Gene Containing the Tobacco Plastid clpP Gene Promoter and 5′ Untranslated Sequence Fused to the ACCase Coding Sequence and Plastid rps16 Gene 3′ Untranslated Sequence in a Vector for Tobacco Plastid Transformation

A cDNA library is screened for the acetylcoenzyme A carboxylase (ACCase) gene (U.S. Pat. No. 5,162,602). A plasmid clone containing the full length ACCase gene cDNA is isolated by standard techniques of molecular cloning. PCR primers are designed for amplification of the mature-size ACCase coding sequence from this plasmid using a top strand primer having a 5′ extension containing an NcoI restriction site inserted at amino acid −1 from the deduced mature protein start, thus creating an ATG start codon at this position, and a bottom strand primer having a 5′ extension containing an XbaI restriction site downstream of the stop codon of the ACCase mature coding sequence in the amplified PCR product. The PCR amplification is performed using the designated primers and plasmid DNA template according to standard protocols. Amplified products are cloned and sequenced and a NcoI-XbaI DNA fragment containing the complete mature ACCase coding sequence is isolated by restriction digest with NcoI and XbaI, electrophoresis on a 0.8% TAE agarose gel, and phenol extraction of the excised band.

A plastid transformation vector containing the clpP promoter driving the mature-sized ACCase gene with the rps16 3′ UTR is constructed by digesting pPH140 with NcoI and XbaI and purifying the fragment containing the vector backbone, 5′ and 3′ plastid integration targeting sequences, aadA selectable marker cassette, and clpP promoter/rps16 3′ UTR expression sequences. This product is ligated in a two-way reaction with the NcoI-XbaI DNA fragment containing the mature-sized ACCase coding sequence isolated as described above.

### Example 43

Biolistic Transformation of the Tobacco Plastid Genome

Seeds of Nicotiana tabacum c.v. ‘Xanthi nc’ were germinated seven per plate in a 1″ circular array on T agar medium and bombarded 12-14 days after sowing with 1 μm tungsten particles (M10, Biorad, Hercules, Calif.) coated with DNA from plasmids pPH143 and pPH145 essentially as described in Svab, Z. and Maliga, P. (1993) PNAS 90, 913-917. Bombarded seedlings were incubated on T medium for two days after which leaves were excised and placed abaxial side up in bright light (350-500 μmol photons/m²/s) on plates of RMOP medium (Svab, Z., Hajdukiewicz, P. and Maliga, P. (1990) PNAS 87, 8526-8530) containing 500 μg/ml spectinomycin dihydrochloride (Sigma, St. Louis, Mo.). Resistant shoots appearing underneath the bleached leaves three to eight weeks after bombardment were subcloned onto the same selective medium, allowed to form callus, and secondary shoots isolated and subcloned. Complete segregation of transformed plastid genome copies (homoplasmicity) in independent subclones was assessed by standard techniques of Southern blotting (Sambrook et al., (1989) Molecular Clonina: A Laboratory Manual, Cold Spring Harbor Laboratory, Cold Spring Harbor). BamHI/EcoRI-digested total cellular DNA (Mettler, I. J. (1987) Plant Mol Biol Reporter 5, 346349) was separated on 1% Tris-borate (TBE) agarose gels, transferred to nylon membranes (Amersham) and probed with ³²P-labeled random primed DNA sequences corresponding to a 6.7 kb BamHI/HindlII DNA fragment from pC8 containing a portion of the rps7/12 plastid targeting sequence. Homoplasmic shoots are rooted aseptically on spectinomycin-containing MS/IBA medium (McBride, K. E. et al. (1994) PNAS 91, 7301-7305) and transferred to the greenhouse.

### Example 44

Assessment of Herbicide Tolerance in Nt-pPH143 and Nt-pPH145 Plastid Transformant Lines

Primary homoplasmic transformant lines transformed with pPH143 (line Nt_pPH143) or with pPH145 (line Nt_pPH145), which were obtained as described in Example 43, were grown to maturity in the greenhouse. Flowers were either: (a) self-pollinated, (b) pollinated with wildtype tobacco (c.v. Xanthi nc), or (c) used as pollen donors to fertilize emasculated flowers of wildtype Xanthi plants. Plastid segregation of the linked spectinomycin resistance marker was verified by uniparental female inheritance of the spectinomycin-resistance phenotype in each transformant line using a minimum of 50 seeds per selection pool derived from either selfed or backcross capsules. Additional self or wildtype backcross (Xanthi pollen parent) seeds were germinated in soil. 36 plants of each line (143 1B-1, 143 1B-4, 143 4A-2, 143 4A-5, 145 7A-5, 145 7A-6, 145 8A-3) plus 36 wildtype Xanthi plants as isogenic controls were grown in separate 6″ clay pots in a controlled environment cubicle. In order to assess tolerance to the protox inhibitor Formula XVII, plants of Xanthi and the seven transformant lines were distributed into eight identical 16-pot flats (2 plants of each type per flat). The flats were sprayed with Formula XVII until runoff at concentrations of either 0, 0.5, 2.5, 5, 10, 25, 50, or 100 mg Formula XVII per liter. Solutions were made up in water using 4 g/liter or 40 g/liter stock solutions of Formula XVII dissolved in dimethylsulfoxide (DMSO) and used immediately after preparation. Twenty microliters of the wetting agent Silwett was added to each 200 ml volume of herbicide solution for a final concentration of 0.01%. Flats were sprayed in the late afternoon and allowed to dry overnight before transfer to the growth cubicle. Tolerance was assesed by comparing leaf damage and wilting to the untransformed Xanthi controls at 0, 18 hrs, 48 hrs, and 6 days post-application. Severe damage was apparent on the Xanthi plants at all concentrations above 0.5 mg/l, and complete wilting/burn down occurred above 2.5 mg/l. Only slight damage occurred on the Nt_PPH143 plants even at the highest concentration (100 mg/liter), and the plants soon outgrew the bleached spots (the appearance of Xanthi at 0.5 mg/liter was approximately equivalent to Nt_pPH143 1B-1 at 100 mg/liter, giving a tolerance of ca. 200-fold).

### Example 45

Plastid Transformation of Maize

Type I embryogenic callus cultures (Green et al. (1983) in A. Fazelahmad, K. Downey, J. Schultz, R. W. Voellmy, eds. Advances in Gene Technology: Molecular Genetics of Plants and Animals. Miami Winter Symposium Series, Vol. 20. Academic Press, N.Y.) of the proprietary genotypes CG00526 and CG0O714 are initiated from immature embryos, 1.5-2.5 mm in length, from greenhouse grown material. Embryos are aseptically excised from surface-sterilized ears approximately 14 days after pollination. Embryos of CG00526 are placed on D callus initiation media with 2% sucrose and 5mg/L chloramben (Duncan et al. (1985) Planta 165: 322-332) while those of CG00714 are placed onto KM callus initiation media with 3% sucrose and 0.75mg/L 2,4-d (Kao and Michayluk (1975) Planta 126, 105-110). Embryos and embryogenic cultures are subsequently cultured in the dark. Embryogenic responses are removed from the explants after ˜14 days. CG00526 responses are placed onto D callus maintenance media with 2% sucrose and 0.5mg/L 2,4-d while those of CG00714 are placed onto KM callus maintenance media with 2% sucrose and 5mg/L Dicamba. After 3 to 8 weeks of weekly selective subculture to fresh maintenance media, high quality compact embryogenic cultures are established. Actively growing embryogenic callus pieces are selected as target tissue for gene delivery. The callus pieces are plated onto target plates containing maintenance medium with 12% sucrose approximately 4 hours prior to gene delivery. The callus pieces are arranged in circles, with radii of 8 and 10 mm from the center of the target plate. Plasmid DNA is precipitated onto gold microcarriers as described in the DuPont Biolistics manual. Two to three μg of each plasmid is used in each 6 shot microcarrier preparation. Genes are delivered to the target tissue cells using the PDS-1000He Biolistics device. The settings on the Biolistics device are as follows: 8 mm between the rupture disc and the macrocarrier, 10 mm between the macrocarrier and the stopping screen and 7 cm between the stopping screen and the target. Each target plate is shot twice using 650psi rupture discs. A 200×200 stainless steel mesh (McMaster-Carr, New Brunswick, N.J.) is placed between the stopping screen and the target tissue.

Five days later, the bombed callus pieces are transferred to maintenance medium with 2% sucrose and 0.5mg/L 2,4-d, but without amino acids, and containing 750 or 1000 nM Formula XVII. The callus pieces are placed for 1 hour on the light shelf 4-5 hours after transfer or on the next day, and stored in the dark at 27° C. for 5-6 weeks. Following the 5-6 week primary selection stage, yellow to white tissue is transferred to fresh plates containing the same medium supplemented with 500 or 750 nM Formula XVII. 4-5 hours after transfer or on the next day, the tissues are placed for 1 hour on the light shelf and stored in the dark at 27° C. for 3-4 weeks. Following the 3-4 week secondary selection stage, the tissues are transferred to plates containing the same medium supplemented with 500 nM Formula XVII. Healthy growing tissue is placed for 1 hour on the light shelf and stored in the dark at 27° C. It is subcultured every two weeks until the colonies are large enough for regeneration.

At that point, colonies are transferred to a modified MS medium (Murashige and Skoog (1962) Physiol. Plant 15: 473-497) containing 3% sucrose (MS3S) with no selection agent and placed in the light. For CG00526, 0.25mg/L ancymidol and 0.5mg/L kinetin are added to this medium to induce embryo germination, while for CG00714, 2 mg/L benzyl adenine is added. Regenerating colonies are transferred to MS3S media without ancymidol and kinetin, or benzyl adenine, for CG00526 or CG00714, respectively, after 2 weeks. Regenerating shoots with or without roots are transferred to boxes containing MS3S medium and small plants with roots are eventually recovered and transferred to soil in the greenhouse.

Various modifications of the invention described herein will become apparent to those skilled in the art. Such modifications are intended to fall within the scope of the appended claims.

What is claimed is:

1. A chimeric gene comprising:
(a) a DNA molecule isolated from a plant, which in its native state encodes a polypeptide that comprises:
(i) a plastid transit peptide, and
(ii) a mature enzyme that is natively targeted to a plastid of said plant by the plastid transit peptide,
wherein said DNA molecule is modified such that it does not encode a functional plastid transit peptide; and
(b) a promoter capable of expressing said DNA molecule in a plastid, wherein said promoter is operatively linked to said DNA molecule.

2. A chimeric gene according to claim 1, wherein said enzyme is naturally inhibited by a herbicidal compound.

3. A chimeric gene according to claim 2, wherein said enzyme has at least one amino acid modification compared to a corresponding naturally occurring enzyme, wherein said at least one amino acid modification confers resistance to an inhibitor of the naturally occurring enzyme.

4. A chimeric gene according to claim 1, wherein said enzyme has protoporphyrinogen oxidase (protox) activity.

5. A chimeric gene according to claim 4, wherein said enzyme has at least one amino acid modification compared to a naturally occurring protox enzyme, wherein said at least one amino acid modification confers resistance to an inhibitor of the naturally occurring protox enzyme.

6. A chimeric gene according to claim 5, wherein said at least one amino acid modification comprises an amino acid substitution occurring at a position corresponding to position 240, 245, 246, 388, 390, 451, 455, 500, or 536 of the comparitive alignment shown in Table 1.

7. A chimeric gene according to claim 6, wherein said at least one amino acid modification further comprises an additional amino acid substitution occuring at a position corresponding to position 143, 274, 330, 450, or 523 of the comparitive alignment shown in Table 1.

8. A chimeric gene according to claim 5, wherein said DNA molecule is isolated from Arabidopsis thaliana.

9. A chimeric gene according to claim 8, wherein said amino acid substitution occurs at a position corresponding to position 215, 220, 221, 363, 365, 426, 430, 475, or 511 of SEQ ID NO:2.

10. A chimeric gene according to claim 9, wherein a cysteine occurring at the position corresponding to position 215 of SEQ ID NO:2 is replaced with phenylalanine, leucine, or lysine.

11. A chimeric gene according to claim 9, wherein an alanine occurring at the position corresponding to position 220 of SEQ ID NO:2 is replaced with valine, threonine, leucine, cysteine, or isoleucine.

12. A chimeric gene according to claim 9, wherein a glycine occurring at the position corresponding to position 221 of SEQ ID NO:2 is replaced with serine or leucine.

13. A chimeric gene according to claim 9, wherein a proline occurring at the position corresponding to position 363 of SEQ ID NO:2 is replaced with serine or histidine.

14. A chimeric gene according to claim 9, wherein a valine occurring at the position corresponding to position 365 of SEQ ID NO:2 is replaced with leucine.

15. A chimeric gene according to claim 9, wherein a tyrosine occurring at the position corresponding to position 426 of SEQ ID NO:2 is replaced with cysteine, isoleucine, leucine, threonine, methionine, valine, alanine, or arginine.

16. A chimeric gene according to claim 9, wherein a serine occurring at the position corresponding to position 430 of SEQ ID NO:2 is replaced with proline.

17. A chimeric gene according to claim 9, wherein a isoleucine occurring at the position corresponding to position 475 of SEQ ID NO:2 is replaced with threonine, histidine, glycine, or asparagine.

18. A chimeric gene according to claim 9, wherein a valine occurring at the position corresponding to position 511 of SEQ ID NO:2 is replaced with alanine.

19. A chimeric gene according to claim 9, wherein said at least one amino acid modification further comprises an additional amino acid substitution occuring at a position corresponding to position 118, 249, 305, 425, or 498 of SEQ ID NO:2.

20. A chimeric gene according to claim 9, wherein said at least one amino acid modification comprises an amino acid substitution occurring at a position corresponding to position 220 or 426 of SEQ ID NO:2 and an additional amino acid substitution occurring at a position corresponding to position 118, 249, 305, 425, or 498 of SEQ ID NO:2.

21. A chimeric gene according to claim 20, wherein a proline occurring at the position corresponding to position 118 of SEQ ID NO:2 is replaced with leucine.

22. A chimeric gene according to claim 20, wherein a threonine occurring at the position corresponding to position 249 of SEQ ID NO:2 is replaced with isoleucine or alanine.

23. A chimeric gene according to claim 20, wherein a serine occurring at the position corresponding to position 305 of SEQ ID NO:2 is replaced with leucine.

24. A chimeric gene according to claim 20, wherein an asparagine occurring at the position corresponding to position 425 of SEQ ID NO:2 is replaced with serine.

25. A chimeric gene according to claim 207, wherein a tyrosine occurring at the position corresponding to position 498 of SEQ ID NO:2 is replaced with cysteine.

26. A chimeric gene according to claim 1, wherein said promoter is isolated from the 5′ flanking region upstream of the coding sequence of a plastid clpP gene.

27. A chimeric gene according to claim 26, wherein said plastid clpP gene is from tobacco.

28. A chimeric gene according to claim 26, wherein said plastid clpP gene is from Arabidopsis thaliana.

29. A chimeric gene according to claim 1, wherein said promoter is isolated from the 5′ flanking region upstream of the coding sequence of a plastid 16S ribosomal RNA operon.

30. A chimeric gene according to claim 1, wherein said DNA molecule is modified in that at least a portion of the native plastid transit peptide coding sequence is absent from said DNA molecule.

31. A chimeric gene according to claim 30, wherein said DNA molecule is modified in that all of the native plastid transit peptide coding sequence is absent from said DNA molecule.

32. A chimeric gene according to claim 1, wherein said DNA molecule is modified in that one or more nucleotides of the native plastid transit peptide coding sequence are mutated, thereby rendering an encoded plastid transit peptide nonfunctional.

33. A plastid transformation vector comprising a chimeric gene according to claim 1.

34. A plastid comprising a plastid transformation vector according to claim 33.

35. A plant, plant tissue, or plant cell, including the progeny thereof, comprising a plastid according to claim 34.

36. A plastid transformation vector comprising a chimeric gene according to claim 3.

37. A plastid comprising a plastid transformation vector according to claim 36.

38. A plant, plant tissue, or plant cell, including the progeny thereof, comprising a plastid according to claim 37.

39. A plastid transformation vector comprising a chimeric gene according to claim 5.

40. A plastid comprising a plastid transformation vector according to claim 39.

41. A plant, plant tissue, or plant cell, including the progeny thereof, comprising a plastid according to claim 40.

42. A method for controlling the growth of undesired vegetation, which comprises applying to a population of a plant according to claim 35 an effective amount of an inhibitor of the enzyme.

43. A method for controlling the growth of undesired vegetation, which comprises applying to a population of a plant according to claim 38 an effective amount of an inhibitor of the enzyme.

44. A method for controlling the growth of undesired vegetation, which comprises applying to a population of a plant according to claim 41 an effective amount of a protox inhibitor.

45. A method of producing a plant that is tolerant to a herbicidal compound in an amount that naturally inhibits plant growth, comprising the steps of:
(a) introducing a chimeric gene according to claim 2 into the plastome of a plant; and
(b) expressing said DNA molecule in the plastids of said plant.

46. A method of producing a plant that is tolerant to a herbicidal compound in an amount that naturally inhibits plant growth, comprising the steps of:
(a) introducing a chimeric gene according to claim 3 into the plastome of a plant; and
(b) expressing said DNA molecule in the plastids of said plant.

47. A method of producing a plant that is tolerant to a herbicidal compound in an amount that naturally inhibits plant growth, comprising the steps of:
(a) introducing a chimeric gene according to claim 5 into the plastome of a plant; and
(b) expressing said DNA molecule in the plastids of said plant.

48. A method for selecting a transplastomic plant cell, comprising the steps of:
(a) introducing a chimeric gene according to claim 1 into the plastome of a plant cell;
(b) expressing the enzyme encoded by said DNA molecule in the plastids of said plant cell; and (c) selecting a cell that is resistant to a herbicidal compound that naturally inhibits the activity of the enzyme encoded by said DNA molecule, whereby the resistant cell comprises transformed plastids.

49. A method for selecting a transplastomic plant cell, comprising the steps of:
(a) introducing a chimeric gene according to claim 3 into the plastome of a plant cell;
(b) expressing the enzyme encoded by said DNA molecule in the plastids of said plant cell; and
(c) selecting a cell that is resistant to a herbicidal compound that naturally inhibits the activity of the enzyme encoded by said DNA molecule, whereby the resistant cell comprises transformed plastids.

50. A method for selecting a transplastomic plant cell, comprising the steps of:
(a) introducing a chimeric gene according to claim 5 into the plastome of a plant cell;
(b) expressing the enzyme encoded by said DNA molecule in the plastids of said plant cell; and
(c) selecting a cell that is resistant to a herbicidal compound that naturally inhibits the activity of the enzyme encoded by said DNA molecule, whereby the resistant cell comprises transformed plastids.
